SEQUENCE LISTING (100 sequences)

SEQ ID NO: 1 - protein, 16 aa, Homo sapiens
  MISC_FEATURE (1)..(16): c-Abl and Bcr-Able switch control ligand
DFGLSRLMTGDTYTAH

SEQ ID NO: 2 - protein, 16 aa, Homo sapiens
  MISC_FEATURE (1)..(16): c-Abl Myristilated Switch Control Ligand Sequence
GQQPGKVLGDQRRPSL

SEQ ID NO: 3 - protein, 360 aa, Homo sapiens
  MISC_FEATURE (1)..(360): p38alpha full length gene product - NM_001315
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHINQLQQIMRLTGTPPAYLINRMPSHEARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 4 - protein, 24 aa, Homo sapiens
  MISC_FEATURE (1)..(24): p38 Switch Control Ligand
DFGLARHTDDEMTGYVATRWYRTY

SEQ ID NO: 5 - protein, 21 aa, Homo sapiens
  MISC_FEATURE (1)..(21): GSK-3b switch control Ligand sequence 1
DFGSQKQLVKGEPNVSYICSK

SEQ ID NO: 6 - protein, 10 aa, Homo sapiens
  MISC_FEATURE (1)..(10): GSK-3b switch control Ligand sequence 2
GRPRTTSFAE

SEQ ID NO: 7 - protein, 21 aa, Homo sapiens
  MISC_FEATURE (1)..(21): IRK1 switch control Ligand sequence 1
DFGMRRSIYETDYYRKGGKGL

SEQ ID NO: 8 - protein, 11 aa, Homo sapiens
  MISC_FEATURE (1)..(11): PKB/Akt1 switch control Ligand sequence 1
PHFPQFSYSAS

SEQ ID NO: 9 - protein, 12 aa, Homo sapiens
  MISC_FEATURE (1)..(12): TGF-betaR1 switch control Ligand sequence 1
TTSGSGSGLPLL

SEQ ID NO: 10 - protein, 293 aa, Homo sapiens
  MISC_FEATURE (1)..(293): 1FPU, A PROTO-ONCOGENE TYROSINE-PROTEIN KINASE ABL chain A)
GAMDPSSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRGT

SEQ ID NO: 11 - protein, 293 aa, Homo sapiens; identical to SEQ ID NO: 10
  MISC_FEATURE (1)..(293): 1IEP, A PROTO-ONCOGENE TYROSINE-PROTEIN KINASE ABL(chain A)

SEQ ID NO: 12 - protein, 537 aa, Homo sapiens
  MISC_FEATURE (1)..(537): c-ABL, 1OPL, Chain A
MGQQPGKVLGDQRRPSLPALHFIKGAGKRDSSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRNLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKENLYFQ

SEQ ID NO: 13 - protein, 537 aa, Homo sapiens; identical to SEQ ID NO: 12
  MISC_FEATURE (1)..(537): c-ABL, 1OPL, Chain B

SEQ ID NO: 14 - protein, 360 aa, Homo sapiens
  MISC_FEATURE (1)..(360): 1KV2 P38alpha MAP KINASE
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 15 - protein, 378 aa, Homo sapiens
  MISC_FEATURE (1)..(378): 1GNG, A GLYCOGEN SYNTHASE KINASE-3 BETA
MHHHHHHHHHHKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSXICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTN

SEQ ID NO: 16 - protein, 352 aa, Homo sapiens
  MISC_FEATURE (1)..(352): 1H8F, Chain A, A GLYCOGEN SYNTHASE KINASE-3 BETA
SKVTTVVATPGQGPDRPQEVSYTDTKVIGNGSFGVVYQAKLCDSGELVAIKKVLQGKAFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFAFPQIKAHPWTKVFRPRTPPEAIALCSRLLEYTPTARLTPLEACAHSFFDELRDPNVKLPNGRDTPALFNFTTQELSSNPPLATILIPPHARIQA

SEQ ID NO: 17 - protein, 352 aa, Homo sapiens; identical to SEQ ID NO: 16
  MISC_FEATURE (1)..(352): 1H8F, Chain A, A GLYCOGEN SYNTHASE KINASE-3 BETA

SEQ ID NO: 18 - protein, 420 aa, Homo sapiens
  MISC_FEATURE (1)..(420): 1I09, Chain A, A GLYCOGEN SYNTHASE KINASE-3 BETA
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 19 - protein, 306 aa, Homo sapiens
  MISC_FEATURE (1)..(306): 1GAG, Chain A, A INSULIN RECEPTOR, TYROSINE KINASE DOMAIN
VFPSSVFVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIXETDXXRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENK

SEQ ID NO: 20 - protein, 13 aa, Homo sapiens
  MISC_FEATURE (1)..(13): 1GAG, Chain B, BISUBSTRATE PEPTIDE INHIBITOR
PATGDFMNMSPVG

SEQ ID NO: 21 - protein, 306 aa, Homo sapiens
  MISC_FEATURE (1)..(306): 1IRK INSULIN RECEPTOR (TYROSINE KINASE DOMAIN) MUTANT WITH CYS 981 REPLACED BY SER AND TYR 984 REPLACED BY PHE (C981S, Y984F) - CHAIN _
VFPSSVFVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENK

SEQ ID NO: 22 - protein, 315 aa, Homo sapiens
  MISC_FEATURE (1)..(315): 1GZK, A RAC-BETA SERINE/THREONINE PROTEIN KINASE
KVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLG

SEQ ID NO: 23 - protein, 315 aa, Homo sapiens; identical to SEQ ID NO: 22
  MISC_FEATURE (1)..(315): 1GZO, A RAC-BETA SERINE/THREONINE PROTEIN KINASE

SEQ ID NO: 24 - protein, 335 aa, Homo sapiens
  MISC_FEATURE (1)..(335): 1GZN, A RAC-BETA SERINE/THREONINE PROTEIN KINASE
KVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIR

SEQ ID NO: 25 - protein, 342 aa, Homo sapiens
  MISC_FEATURE (1)..(342): 1B6C B TGF-B SUPERFAMILY RECEPTOR TYPE I, Chain B
EDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 26 - protein, 107 aa, Homo sapiens
  MISC_FEATURE (1)..(107): 1B6C - FK506-BINDING PROTEIN, Chain A
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 27 - protein, 350 aa, Homo sapiens
  MISC_FEATURE (1)..(350): 1O9U, A GLYCOGEN SYNTHASE KINASE-3 BETA
SKVTTVVATPGQGPDRPQEVSYTDTKVIGNGSFGVVYQAKLCDSGELVAIKKVLQGKAFKNRELQIMRKLDHCNIVRLRYFFYSSGEKKDEVYLNLVLDYVPATVYRVARHYSRAKQTLPVIYVKLYMYQLFRSLAYIHSFGICHRDIKPQNLLLDPDTAVLKLCDFGSAKQLVRGEPNVSXICSRYYRAPELIFGATDYTSSIDVWSAGCVLAELLLGQPIFPGDSGVDQLVEIIKVLGTPTREQIREMNPNYTEFAFPQIKAHPWTKVFRPRTPPEAIALCSRLLEYTPTARLTPLEACAHSFFDELRDPNVKLPNGRDTPALFNFTTQELSSNPPLATILIPPHARI

SEQ ID NO: 28 - protein, 18 aa, Homo sapiens
  MISC_FEATURE (1)..(18): 1O9U, B AXIN PEPTIDE
VEPQKFAEELIHRLEAVQ

SEQ ID NO: 29 - protein, 1149 aa, Homo sapiens
  MISC_FEATURE (1)..(1149): c-abl Homo sapiens NM_007313 Homo sapiens v-abl Abelson murine leukemia viral oncogene homolog 1 (ABL1), transcript variant b, mRNA.
MGQQPGKVLGDQRRPSLPALHFIKGAGKKESSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 30 - protein, 360 aa, Homo sapiens; identical to SEQ ID NO: 3
  MISC_FEATURE (1)..(360): p38alpha full length gene product - NM_001315

SEQ ID NO: 31 - protein, 433 aa, Homo sapiens
  MISC_FEATURE (1)..(433): Full length GSK-3b - NM_002093
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 32 - protein, 378 aa, Homo sapiens; identical to SEQ ID NO: 15
  MISC_FEATURE (1)..(378): 1GNG, Chain A, A GLYCOGEN SYNTHASE KINASE-3 BETA

SEQ ID NO: 33 - protein, 2029 aa, Homo sapiens
  MISC_FEATURE (1)..(2029): Full length Bcr-Abl
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTELLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSLQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 34 - protein, 1382 aa, Homo sapiens
  MISC_FEATURE (1)..(1382): IRK-1 (full length) - NM_000208
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNHIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTTTQGKLFFHYNPKLCLSEIHKMEEVSGTKGRQERNDIALKTNGDKASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 35 - protein, 306 aa, Homo sapiens; identical to SEQ ID NO: 19
  MISC_FEATURE (1)..(306): 1GAG, Chain A, A INSULIN RECEPTOR, TYROSINE KINASE DOMAIN

SEQ ID NO: 36 - protein, 481 aa, Homo sapiens
  MISC_FEATURE (1)..(481): Full length PKB/AKT, consensus sequence
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 37 - protein, 335 aa, Homo sapiens; identical to SEQ ID NO: 24
  MISC_FEATURE (1)..(335): PKB/AKT Kinase only domain

SEQ ID NO: 38 - protein, 390 aa, Homo sapiens
  MISC_FEATURE (1)..(390): Full length gene of TGF-B1, NM_000660
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 39 - protein, 503 aa, Homo sapiens
  MISC_FEATURE (1)..(503): TGF-BR1 receptor kinase, NM_004612
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 40 - protein, 765 aa, Homo sapiens
  MISC_FEATURE (1)..(765): Braf Construct 1 - Full length Gene
MAALSGGGGGGAEPGQALFNGDMEPEAGAGRPAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 41 - protein, 30 aa, Homo sapiens
  MISC_FEATURE (1)..(30): Braf switch control ligand sequence
DFGLATVKSRWSGSHQFEQLSGSILWMAPE

SEQ ID NO: 42 - protein, 765 aa, Homo sapiens
  MISC_FEATURE (1)..(765): Braf Construct 2 - Full length Gene, V599E mutant
MAALSGGGGGGAEPGQALFNGDMEPEAGAGRPAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATEKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 43 - protein, 30 aa, Homo sapiens
  MISC_FEATURE (1)..(30): Braf (V599E) switch control ligand sequence
DFGLATEKSRWSGSHQFEQLSGSILWMAPE

SEQ ID NO: 44 - protein, 291 aa, Homo sapiens
  MISC_FEATURE (1)..(291): apo Braf, residues 432-723, Kinase domain only (33 kD)
EDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPK

SEQ ID NO: 45 - protein, 291 aa, Homo sapiens
  MISC_FEATURE (1)..(291): apo Braf (V599E), residues 432-723 (33 kD)
EDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATEKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPK

SEQ ID NO: 46 - protein, 291 aa, Homo sapiens; identical to SEQ ID NO: 45
  MISC_FEATURE (1)..(291): apo Braf(v599E, residues 432-723, specifically isotopically 13C/15N labeled at glutamic acid 500 (bolded)

SEQ ID NO: 47 - protein, 291 aa, Homo sapiens; identical to SEQ ID NO: 45
  MISC_FEATURE (1)..(291): apo Braf(v599E), residues 432-723, specifically isotopically 13C/15N labeled at phenylalanine 594 (underlined)

SEQ ID NO: 48 - protein, 379 aa, Homo sapiens
  MISC_FEATURE (1)..(379): p38alpha (deCODE construct 1)
MGSSHHHHHHSSGLVPRGSHMSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 49 - protein, 378 aa, Homo sapiens
  MISC_FEATURE (1)..(378): GSK-3b, 1GNG, deCODE construct
MHHHHHHHHHHKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTN

SEQ ID NO: 50 - protein, 374 aa, Homo sapiens
  MISC_FEATURE (1)..(374): GSK-3b, 35-385, deCODE construct
MEEYMPTEHHHHHHENLYFQGTSSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQ

SEQ ID NO: 51 - protein, 556 aa, Homo sapiens
  MISC_FEATURE (1)..(556): Abl 1-531, Y412F mutant (deCODE)
MSYYHHHHHHDYDIPTTENLYFQGAMDPSMGQQPGKVLGDQRRPSLPALHFIKGAGKKESSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTFTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDERGT

SEQ ID NO: 52 - protein, 367 aa, Homo sapiens
  MISC_FEATURE (1)..(367): p38gamma - 1CM8 A PHOSPHORYLATED MAP KINASE P38-GAMMA
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFTDFYLVMPFMGTDLGKLMKHEKLGEDRIQFLVYQMLKGLRYIHAAGIIHRDLKPGNLAVNEDCELKILDFGLARQADSEMXGXVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 53 - protein, 300 aa, Homo sapiens
  MISC_FEATURE (1)..(300): Abl 2 (deCODE)
MSYYHHHHHHDYDIPTTENLYFQGAMDPSSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMRGT

SEQ ID NO: 54 - protein, 445 aa, Homo sapiens
  MISC_FEATURE (1)..(445): gsk-3b, 1H8F (deCODE sequence, construct 1)
MSYYHHHHHHDYDIPTTENLYFQGAMSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 55 - protein, 370 aa, Homo sapiens
  MISC_FEATURE (1)..(370): Roche sequence for phospho-p38
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMESGGGSHHHHHH

SEQ ID NO: 56 - protein, 316 aa, Homo sapiens
  MISC_FEATURE (1)..(316): Abl1 sequence
MSYYHHHHHHDYDIPTTENLYFQGAMDPSSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRGT

SEQ ID NO: 57 - protein, 253 aa, Homo sapiens
  MISC_FEATURE (1)..(253): PROCASPASE-7 structure 1K88 (chain A) - 1K88
TRDRVPTYQYNMNFEKLGKCIIINNKNFDKVTGMGVRNGTDKDAEALFKCFRSLGFDVIVYNDCSCAKMQDLLKKASEEDHTNAACFACILLSHGEENVIYGKDGVTPIKDLTAHFRGARCKTLLEKPKLFFIQAARGTELDDGIQADSGPINDTDANPRYKIPVEADFLFAYSTVPGYYSWRSPGRGSWFVQALCSILEEHGKDLEIMQILTRVNDRVARHFESQSDDPHFHEKKQIPCVVSMLTKELYFSQ

SEQ ID NO: 58 - protein, 307 aa, Homo sapiens
  MISC_FEATURE (1)..(307): BRAF 2 sequence (deCODE)
MDRGSHHHHHHGSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATEKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHR

SEQ ID NO: 59 - protein, 2050 aa, Homo sapiens [sequence omitted]

SEQ ID NO: 60 - protein, 2050 aa, Homo sapiens
  MISC_FEATURE (1)..(2050): Bcr-Abl, Y412F
MEEYMPTEHHHHHHENLYFQGMVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTELLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSLQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTFTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 61 - protein, 307 aa, Homo sapiens
  MISC_FEATURE (1)..(307): BRAF 2 sequence (deCODE)
MDRGSHHHHHHGSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHR

SEQ ID NO: 62 - protein, 30 aa, Homo sapiens
DFGLATVKSRWSGSQQVEQPTGSVLWMAPE

SEQ ID NO: 63 - protein, 29 aa, Homo sapiens
DFGLSRLMTGDTYTAHAGAKFPIKWTAPE

SEQ ID NO: 64 - protein, 30 aa, Homo sapiens
DFGLARDIMSDSNYVVRGNARLPVKWMAPE

SEQ ID NO: 65 - protein, 30 aa, Homo sapiens
DFGLARLLDIDETEYHADGGKVPIKWMALE

SEQ ID NO: 66 - protein, 30 aa, Homo sapiens
DFGMTRDIYETDYYRKGGKGLLPVRWMAPE

SEQ ID NO: 67 - protein, 30 aa, Homo sapiens
DFGLARDIYKDPDYVRKGDARLPLKWMAPE

SEQ ID NO: 68 - protein, 30 aa, Homo sapiens
DFGLARDIKNDSNYVVKGNARLPVKWMAPE

SEQ ID NO: 69 - protein, 32 aa, Homo sapiens
DFGLARDMYDKEYYSVHNKTGAKLPVKWMALE

SEQ ID NO: 70 - protein, 25 aa, Homo sapiens
DFGLARHTDDEMTGYVATRWYRAPE

SEQ ID NO: 71 - protein, 26 aa, Homo sapiens
DFGWSVHAPSSRRTTLCGTLDYLPPE

SEQ ID NO: 72 - protein, 29 aa, Homo sapiens
DFGLARLIKDDEYNPCQGSKFPIKWTAPE

SEQ ID NO: 73 - protein, 27 aa, Homo sapiens
DFGSAKQLVRGEPNVSYICSRYYRAPE

SEQ ID NO: 74 - protein, 27 aa, Homo sapiens
DFGSAKQLVRGEPNVSYICSRYYRAPE

SEQ ID NO: 75 - protein, 19 aa, Homo sapiens
DFGCNSILPPKKDLWTAPE

SEQ ID NO: 76 - protein, 30 aa, Homo sapiens
DFGLSNCAGILGYSDPFSTQCGSPAYAAPE

SEQ ID NO: 77 - protein, 33 aa, Homo sapiens
DFGLFGISGVVREGRRENQLKLSHDWLCYLAPE

SEQ ID NO: 78 - protein, 27 aa, Homo sapiens
DFGSCRSVYSKQPYTEYISTRWYRAPE

SEQ ID NO: 79 - protein, 31 aa, Homo sapiens
DFGCSEKLEDLLCFQTPSYPLGGTYTHRAPE

SEQ ID NO: 80 - protein, 28 aa, Homo sapiens
DFGLCKENIEHNSTTSTFCGTPEYLAPE

SEQ ID NO: 81 - protein, 31 aa, Homo sapiens
DFGLMRALPQNDDHYVMQEHRKVPFAWCAPE

SEQ ID NO: 82 - protein, 28 aa, Homo sapiens
DFGLCKEGIKDGATMKTFCGTPEYLAPE

SEQ ID NO: 83 - protein, 26 aa, Homo sapiens
DFGWSVHAPSLRRKTMCGTLDYLPPE

SEQ ID NO: 84 - protein, 27 aa, Homo sapiens
DFGLSKMEDPGSVLSTACGTPGYVAPE

SEQ ID NO: 85 - protein, 30 aa, Homo sapiens
DFGLATVFRYNNRERLLNKMCGTLPYVAPE

SEQ ID NO: 86 - protein, 32 aa, Homo sapiens
DFGMARGLCTSPAEHQYFMTEYVATRWYRAPE

SEQ ID NO: 87 - protein, 28 aa, Homo sapiens
DFGLCKEGIKDGATMKTFCGTPEYLAPE

SEQ ID NO: 88 - protein, 31 aa, Homo sapiens
DFGASKRLQTICLSGTGMKSVTGTPYWMSPE

SEQ ID NO: 89 - protein, 28 aa, Homo sapiens
DFGLCKESIHDGTVTHTFCGTIEYMAPE

SEQ ID NO: 90 - protein, 28 aa, Homo sapiens
DFGLSKESIDHEKKAYSFCGTVEYMAPE

SEQ ID NO: 91 - protein, 30 aa, Homo sapiens
DFGLTRLLDDFDGTYETQGGKIPIRWTAPE

SEQ ID NO: 92 - protein, 31 aa, Homo sapiens
DFGLARIADPEHDHTGFLTEYVATRWYRAPE

SEQ ID NO: 93 - protein, 29 aa, Homo sapiens
DFGLARVIEDNEYTAREGAKFPIKWTAPE

SEQ ID NO: 94 - protein, 31 aa, Homo sapiens
DFGLTKAIETDKEYYTVKDDRDSPVFWYAPE

SEQ ID NO: 95 - protein, 29 aa, Homo sapiens
DFGLARLIEDNEYTAREGAKFPIKWTAPE

SEQ ID NO: 96 - protein, 29 aa, Homo sapiens
DFGLARVIEDNEYTAREGAKFPIKWTAPE

SEQ ID NO: 97 - protein, 29 aa, Homo sapiens
DFGLARLIEDNEYTARQGAKFPIKWTAPE

SEQ ID NO: 98 - protein, 31 aa, Homo sapiens
DFGLSKALRADENYYKAQTHGKWPVKWYAPE

SEQ ID NO: 99 - protein, 29 aa, Homo sapiens
DFGLARLIEDNEYTARQGAKFPIKWTAPE

SEQ ID NO: 100 - protein, 31 aa, Homo sapiens
DFGLSKALGADDSYYTARSAGKWPLKWYAPE

(19) United States
(12) Patent Application Publication
(10) Pub. No.: US 2008/0220497 A1
(43) Pub. Date: Sep. 11, 2008

(54) Modulation of protein functionalities

(76) Inventors: Daniel L. Flynn, Lawrence, KS (US); Peter A. Petillo, Arlington, MA (US)

Correspondence Address: HOVEY WILLIAMS LLP, 10801 Mastin Boulevard, Suite 1000, Overland Park, KS, 66210, US

(21) Appl. No.: 11/178,834

(22) Filed: Jul. 11, 2005

Related U.S. Application Data
(63) Continuation-in-part of application No. 10/746,545, filed on Dec. 24, 2003.
(60) Provisional application No. 60/638,987, filed on Dec. 23, 2004.
(60) Provisional application No. 60/639,087, filed on Dec. 23, 2004.
(60) Provisional application No. 60/638,986, filed on Dec. 23, 2004.
(60) Provisional application No. 60/638,968, filed on Dec. 23, 2004.

(51) Int. Cl. C12N 9/12; C07K 14/00; C12N 9/16; C12N 9/48; C12N 9/10

(52) U.S. Cl. 435/194; 530/402; 435/196; 435/212; 435/193

(57) ABSTRACT

New methods for the rational identification of molecules capable of interacting with specific naturally occurring proteins are provided, in order to yield new pharmacologically important compounds and treatment modalities. Broadly, the method comprises the steps of identifying a switch control ligand forming a part of a particular protein of interest, and also identifying a complemental switch control pocket forming a part of the protein and which interacts with said switch control ligand. The ligand interacts in vivo with the pocket to regulate the conformation and biological activity of the protein such that the protein assumes a first conformation and a first biological activity upon the ligand-pocket interaction, and assumes a second, different conformation and biological activity in the absence of the ligand-pocket interaction. Next, respective samples of said protein in the first and second conformations are provided, and these are screened against one or more candidate molecules by contacting the molecules and the samples. Thereupon, small molecules which bind with the protein at the region of the pocket maybe identified. Novel protein-modulator adducts and methods of altering protein activity are also provided.

### CROSS-REFERENCE TO RELATED APPLICATIONS

This application is a continuation-in-part of identically titled application Ser. No. 10/746,545, filed Dec. 24, 2003, the latter application being incorporated by reference herein. This application claims the priority benefit of the following provisional patent applications: U.S. Patent Application Nos. 60/638,987, filed Dec. 23, 2004, Enzyme Modulators For Treatment Of Inflammatory, Autoimmune, Cardiovascular And Immunological Diseases; 60/639,087, filed Dec. 23, 2004, Enzyme Modulators For Treatment Of Cancers And Hyperproliferative Diseases; 60/638,986, filed Dec. 23, 2004, Enzyme Modulators For Treatment Of Cancers, Hyperproliferative Disorders, Or Diseases Treatable With An Anti-Angiogenic Agent; and 60/638,968, filed Dec. 23, 2004, Enzyme Modulators For Treatment Of Cancers And Hyperproliferative Diseases. Each of these applications is incorporated by reference herein.

Each of the following applications is incorporated by reference: Process For Modulating Protein: Function, Ser. No. 60/437,487, filed Dec. 31, 2002; Anti-Cancer Medicaments, Ser. No. 60/437,403, filed Dec. 31, 2002; Anti-Inflammatory Medicaments, Ser. No. 60/437,415, filed Dec. 31, 2002; Anti-Inflammatory Medicaments, Ser. No. 60/437,304, filed Dec. 31, 2002; and Medicaments For the Treatment of Neurodegenerative Disorders or Diabetes, Ser. No. 60/463,804, filed Apr. 18, 2003.

### BACKGROUND OF THE INVENTION

1. Sequence Listing

The following application contains a sequence listing in computer readable format (CRF). The content of the enclosed CRF is hereby incorporated by reference.

2. Field of the Invention

The present invention is broadly concerned with new, rationalized methods of identifying molecules which serve as protein activity modulators, as well as new protein-modulator adducts. More particularly, the invention is concerned with such methods and adducts which, in preferred forms, make use of a mechanism of protein conformation change involving interaction between switch control ligands and complemental switch control pockets.

3. Description of the Prior Art

Basic research has recently provided the life sciences community with an unprecedented volume of information of the human genetic code, and the proteins that are produced by it. In 2001, the complete sequence of the human genome was reported (Lander, E. S. et al., Initial Sequencing and Analysis of the Human Genome; Nature (2001) 409:860; Venter, J. C. et al., The Sequence of the Human Genome, Science (2001) 291:1304). The global research community is now classifying the 50,000+ proteins that are encoded by this genetic sequence, and more importantly, it is attempting to identify those proteins that are causative of major, under-treated human diseases. Despite the wealth of information that the human genome and its proteins are providing, particularly in the area of conformational control of protein function, the methodology and strategy by which the pharmaceutical industry sets about to develop small molecule therapeutics has not significantly advanced beyond using native protein binding sites for binding to small molecule therapeutic agents. These native sites are normally used by proteins to perform essential cellular functions by binding to and processing natural substrates or transducing signals from natural ligands. Because these native sites are used broadly by many other proteins within protein families, drugs which interact with them are often plagued by lack of selectivity and, as a consequence, insufficient therapeutic windows to achieve maximum efficacy. Side effects and toxicities are revealed in such small molecules, either during preclinical discovery, clinical trials, or later in the marketplace. Side effects and toxicities continue to be a major reason for the high attrition rate seen within the drug development process. For the kinase protein family of proteins, interactions at these native sites have been recently reviewed: see J. Dumas, Emerging Pharmacophores: 1997-2000, Expert Opinion on Therapeutic Patents (2001) 11: 405-429; J. Dumas, Editor, Current Topics in Medicinal Chemistry (2002) 2: issue 9.

It is known that proteins are flexible, and this flexibility has been reported and utilized with the discovery of the small molecules which bind to alternative, flexible active sites with proteins. For a review of this topic, see Teague, Nature Reviews/Drug Discovery, Vol. 2, pp. 527-541 (2003). See also, Wu et al., Structure, Vol. 11, pp. 399-410 (2003). However these reports focus on small molecules which bind only to proteins at the protein natural active sites. Peng et al., Bio. Organic and Medicinal Chemistry Ltrs., Vol. 13, pp. 3693-3699 (2003), and Schindler, et al., Science, Vol. 289, p. 1938 (2000) describe inhibitors of Abl kinase. These inhibitors are identified in WO Publication No. 2002/034727. This class of inhibitors binds to the ATP active site while also binding in a mode that induces movement of the kinase catalytic loop. Pargellis et al., Nature Structural Biology, Vol. 9, p. 268 (2002) reported inhibitors of p38 alpha-kinase which were also disclosed in WO Publication No. 00/43384 and Regan et al., J. Medicinal Chemistry, Vol. 45, pp. 2994-3008 (2002). This class of inhibitors also interacts with the kinase at the ATP active site involving a concomitant movement of the kinase activation loop.

More recently, it has been disclosed that kinases utilize activation loops and kinase domain regulatory pockets to control their states of catalytic activity. This has been recently reviewed: see M. Huse and J. Kuriyan, Cell (2002) 109: 275.

### SUMMARY OF THE INVENTION

The present invention is directed to methods of identifying molecules which interact with specific naturally occurring proteins (e.g., mammalian, and especially human proteins) in order to modulate the activity of the proteins, as well as novel protein-small molecule modulator adducts. In its method aspects, the invention exploits a characteristic of naturally occurring proteins, namely that the proteins change their conformations in vivo with a corresponding alteration in protein activity. For example, a given protein in one conformation may be biologically upregulated as an enzyme, while in another conformation, the same protein may be biologically downregulated. Moreover, the invention preferably makes use of one mechanism of conformation change utilized by naturally occurring proteins, through the interaction of what are termed “switch control ligands” and complemental “switch control pockets” within the protein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic representation of a naturally occurring mammalian protein in accordance with the invention including “on” and “off” switch control pockets, a transiently modifiable switch control ligand, and an active ATP site;

FIG. 2 is a schematic representation of the protein of FIG. 1, wherein the switch control ligand is illustrated in a binding relationship with the off switch control pocket, thereby causing the protein to assume a first biologically downregulated conformation;

FIG. 3 is a view similar to that of FIG. 1, but illustrating the switch control ligand in its charged-modified condition wherein the OH groups of certain amino acid residues have been phosphorylated;

FIG. 4 is a view similar to that of FIG. 2, but depicting the protein wherein the switch control ligand is in a binding relationship with the on switch control pocket, thereby causing the protein to assume a second biologically-active conformation different than the first conformation of FIG. 2;

FIG. 4a is an enlarged schematic view illustrating a representative binding between the phosphorylated residues of the switch control ligand, and complemental residues from the on switch control pocket;

FIG. 4b is an enlarged schematic view illustrating a representative binding between a switch control ligand and the on-pocket of a protein, wherein the switch control ligand of the protein contains methionine amino acid residues modified by oxidation to sulfoxide species;

FIG. 4c is an enlarged schematic view illustrating a representative binding between a switch control ligand and the on-pocket of a protein, wherein the switch control ligand of the protein contains methionine amino acid residues modified by oxidation to sulfone species;

FIG. 4d is an enlarged schematic view illustrating a representative binding between a switch control ligand and the on-pocket of a protein, wherein the switch control ligand of the protein contains cysteine amino acid residues modified by oxidation to sulfenic acid species;

FIG. 4e is an enlarged schematic view illustrating a representative binding between a switch control ligand and the on-pocket of a protein, wherein the switch control ligand of the protein contains cysteine amino acid residues modified by oxidation to sulfonic acid species;

FIG. 4f is an enlarged schematic view illustrating a representative binding between a switch control ligand and the on-pocket of a protein, wherein the switch control ligand of the protein contains cysteine amino acid residues modified by nitrosylation to S-nitrosylated species;

FIG. 5 is a view similar to that of FIG. 1, but illustrating in schematic form possible small molecule compounds in a binding relationship with the on and off switch control pockets;

FIG. 6 is a schematic view of the protein in a situation where a composite switch control pocket is formed with portions of the switch control ligand and the on switch control pocket, and with a small molecule in binding relationship with the composite pocket;

FIG. 6a is a schematic view of the protein in a situation where a composite on switch control pocket is formed with a first portion of the switch control ligand 106a and the on switch control pocket, and with a small molecule in binding relationship with the composite on switch control pocket, and wherein a second portion of the switch control ligand 106b is in binding relationship with the off switch control pocket;

FIG. 7 is a schematic view of the protein in a situation where a combined switch control pocket is formed with portions of the on switch control pocket, the switch control ligand sequence, and the active ATP site, and with a small molecule in binding relationship with the combined switch control pocket;

FIG. 8 is a X-ray crystal structural ribbon diagram illustrating the on conformation of the insulin receptor kinase having SEQ ID NO. 19 in its biologically upregulated state;

FIG. 9 is a similar to FIG. 8 but depicts the insulin receptor kinase having SEQ ID NO. 21 in the off conformation in its biologically downregulated state;

FIG. 10 is a SURFNET visualization of Abl kinase having SEQ ID NO. 10, with the on switch control pocket illustrated in blue;

FIG. 11 is a GRASP visualization of Abl kinase having SEQ ID NO. 10, with the on switch control pocket encircled in yellow;

FIG. 12 is a ribbon diagram of the Abl kinase protein having SEQ ID NO. 10, with important amino acid residues of the on switch control pocket identified;

FIG. 13 is a ribbon diagram of the Abl kinase having SEQ ID NO. 10 illustrating the combined switch control pocket (on switch control pocket/switch control ligand sequence/ATP active site);

FIG. 14 is a SURFNET visualization of p38 kinase having SEQ ID NO. 14 with the on switch control pocket illustrated in blue;

FIG. 15 is a GRASP visualization of p38 kinase SEQ ID NO. 14 with the on switch control pocket encircled in yellow;

FIG. 16 is a ribbon diagram of p38 kinase SEQ ID NO. 52 with important amino acid residues of the on switch control pocket identified;

FIG. 17 is a SURFNET visualization of Gsk-3 beta kinase SEQ ID NO. 16 with the dual functionality on-off switch control pocket illustrated in blue;

FIG. 18 is a GRASP visualization of Gsk-3 beta kinase SEQ ID NO. 16 with the dual functionality on-off switch control pocket encircled in yellow;

FIG. 19 is ribbon diagram of Gsk-3 beta kinase SEQ ID NO. 16 with important amino acid residues of the combination on-off switch control pocket identified;

FIG. 20 is a SDS-PAGE gel identifying the semi-purified Abl kinase SEQ ID NO. 53 in its unphosphorylated state;

FIG. 21 is a SDS-PAGE gel identifying the purified Abl kinase SEQ ID NO. 53 in its unphosphorylated state;

FIG. 22 is the chromatogram elution profile of semi-purified Abl kinase SEQ ID NO. 53;

FIG. 23 is the chromatogram elution profile of purified Abl kinase SEQ ID NO. 53;

FIG. 24 is an SDS-PAGE gel identifying Abl kinase SEQ ID NO. 53 before (lanes 2-4) and after (lanes 5-8) TEV tag cleavage;

FIG. 25 is a UV spectrum of purified Abl kinase having SEQ ID NO. 53 with the small molecule inhibitor PD 180970 bound to the ATP site of the protein;

FIG. 26 is the chromatogram elution profile of Abl kinase having SEQ ID NO. 51 (Abl 1-531, Y412F mutant) upon purification through Nickel affinity chromatography and Q-Sepharose chromatography;

FIG. 27 is a SDS-PAGE gel of purified Abl kinase having SEQ ID NO. 51;

FIG. 28 is the chromatogram elution profile of purified p38-alpha kinase having SEQ ID NO. 48 in its unphosphorylated state;

FIG. 29 is a SDS-PAGE gel of purified p38-alpha kinase having SEQ ID NO. 48 in its unphosphorylated state;

FIG. 30 is a mass spectrogram of activated Gsk 3-beta kinase having SEQ ID NO. 54 in its phosphorylated state;

FIG. 31 is a mass spectrogram of unactivated Gsk 3-beta kinase having SEQ ID NO. 54 in its unphosphorylated state;

FIG. 32 is a Western Blot analysis staining of phosphorylated Gsk 3-beta kinase having SEQ ID NO. 54 with the anti-phosphotyrosine antibody;

FIG. 33 is a Western Blot analysis staining of unphosphorylated Gsk 3-beta kinase having SEQ ID NO. 54 with the anti-phosphotyrosine antibody;

FIG. 34 is a an X-ray co-crystal structure of the p38-alpha small molecule switch control inhibitor of Example 8 bound into the composite on switch control pocket of p38-alpha kinase having SEQ ID NO. 48;

FIG. 35 is a an X-ray co-crystal structure of the p38-alpha small molecule switch control inhibitor of Example 29 bound into the composite on switch control pocket of p38-alpha kinase having SEQ ID NO. 48;

FIG. 36 is a an X-ray co-crystal structure of the p38-alpha small molecule switch control inhibitor of Example 61 bound into the composite on switch control pocket of p38-alpha kinase having SEQ ID NO. 48;

FIG. 37 is a an X-ray co-crystal structure of the p38-alpha small molecule switch control inhibitor of Example 62 bound into the composite on switch control pocket of p38-alpha kinase having SEQ ID NO. 48;

FIG. 38 is a an X-ray co-crystal structure of the p38-alpha small molecule switch control inhibitor of Example 63 bound into the composite on switch control pocket of p38-alpha kinase having SEQ ID NO. 48;

FIG. 39 is a an X-ray co-crystal structure of the p38-alpha small molecule switch control inhibitor of Example 29 bound into the composite on switch control pocket of doubly phosphorylated p38-alpha kinase having SEQ ID NO. 55;

FIG. 40 is a an X-ray co-crystal structure of the Abl small molecule switch control inhibitor of Example 64 bound into the composite on switch control pocket of Abl kinase having SEQ ID NO. 53;

FIG. 41 is a an X-ray co-crystal structure of the Abl small molecule switch control inhibitor of Example 65 bound into the composite on switch control pocket of Abl kinase having SEQ ID NO. 53;

FIG. 42 is a an X-ray co-crystal structure of the Braf small molecule switch control inhibitor of Example 65 bound into the composite on switch control pocket of oncogenic V599E Braf kinase having SEQ ID NO. 45;

FIG. 43 is a depiction of the fluorescence affinity assay for p38-alpha kinase;

FIG. 44 is a depiction of the fluorescence affinity assay for Abl kinase;

FIG. 45 is a graph of the time-dependent binding of an ATP-competitive binding fluoroprobe to Abl kinase having SEQ ID NO. 56;

FIG. 46 is a graph illustrating the enhancement of binding of a known ATP-competitive binding fluoroprobe to Abl kinase having SEQ ID NO. 56, in the presence of the switch control inhibitor of Example 66, over a period of 100 minutes;

FIG. 47 is a graph similar to that of FIG. 46, illustrating the binding enhancement at the early time period of 0-20 minutes;

FIG. 48 is a graph depicting the EC₅₀ value of the Abl kinase switch inhibitor of Example 64 for accelerating the binding of the known ATP-competitive binding fluoroprobe;

FIG. 48A is a graph depicting the EC₅₀ value of the Abl kinase switch inhibitor of Example 65 for accelerating the binding of the known ATP-competitive binding fluoroprobe;

FIG. 48B is a graph depicting the EC₅₀ value of the Abl kinase switch inhibitor of Example 66 for accelerating the binding of the known ATP-competitive binding fluoroprobe;

FIG. 49 is an X-ray crystal structure of procaspase-7 having SEQ ID NO. 57 illustrating the switch control pocket at the dimer interface;

FIG. 50 is an X-ray crystal structure of the composite switch control pocket of wild-type Braf kinase having SEQ ID NO. 44;

FIG. 51 is a rendering of an SDS-PAGE gel of purified oncogenic V599E Braf kinase having SEQ ID NO. 58; and

FIG. 52 is a graph illustrating the ATP non-competitive type inhibition of p38-alpha kinase having SEQ ID NO. 48 exhibited by the small molecule switch inhibitor of Example 72.

### DETAILED DESCRIPTION OF THE PREFERRED EMBODIMENTS

The present invention provides a way of rationally developing new small molecule modulators which interact with naturally occurring proteins (e.g., mammalian, and especially human proteins) in order to modulate the activity of the proteins. Novel protein-small molecule adducts are also provided. The invention preferably makes use of naturally occurring proteins having a conformational property whereby the proteins change their conformations in vivo with a corresponding change in protein activity. For example, a given enzyme protein in one conformation may be biologically upregulated, while in another conformation, the same protein may be biologically downregulated. The invention preferably makes use of one mechanism of conformation change utilized by naturally occurring proteins, through the interaction of what are termed “switch control ligands” and complemental “switch control pockets” within the protein.

As used herein, “switch control ligand” means a region or domain within a naturally occurring protein and having one or more amino acid residues therein which are modifiable (either transiently (reversibly) or substantially permanently) in vivo between individual states by genomic, biochemical or chemical modification, typically involving mutation, phosphorylation, sulfation, fatty acid acylation, glycosylation, prenylation, carboxylation, nitrosylation, cystinylation (wherein two proximal cysteine residues form a disulfide bond between them) or oxidation of the modifiable ligand residues. Genomic modification, fatty acid acylation, glycosylation, prenylation, or carboxylation constitute substantially permanent modifications of switch control ligand residues, whereas phosphorylation, sulfation, nitrosylation, cystinylation, or oxidation constitute reversible modifications of switch control ligand residues. Similarly, “switch control pocket” means a plurality of contiguous or non-contiguous amino acid residues within a naturally occurring protein and comprising conformational control residues (hereafter referred to as “Z” residues in the case of an on pocket, and “X” residues in the case of an off pocket) capable of binding in vivo with the modifiable residues of a switch control ligand in one of the individual states thereof in order to induce or restrict the conformation of the protein and thereby modulate the biological activity of the protein, and wherein at least some of said conformational control residues are capable of binding with a non-naturally occurring switch control modulator molecule to induce or restrict a protein conformation and thereby modulate the biological activity of the protein.

A protein-modulator adduct in accordance with the invention comprises a naturally occurring protein having a switch control pocket with a non-naturally occurring molecule bound to the protein at the region of said switch control pocket, said molecule being bound to some or all of the conformational control amino acid residues forming a part of the switch control pocket, and serving to at least partially regulate the biological activity of said protein by inducing or restricting the conformation of the protein. Preferably, the protein also has a corresponding switch control ligand embedded within the sequence of the protein, the ligand interacting in vivo with the pocket to regulate the conformation and biological activity of the protein such that the protein will assume a first conformation and a first biological activity upon the ligand-pocket interaction, and will assume a second, different conformation and biological activity in the absence of the ligand-pocket interaction.

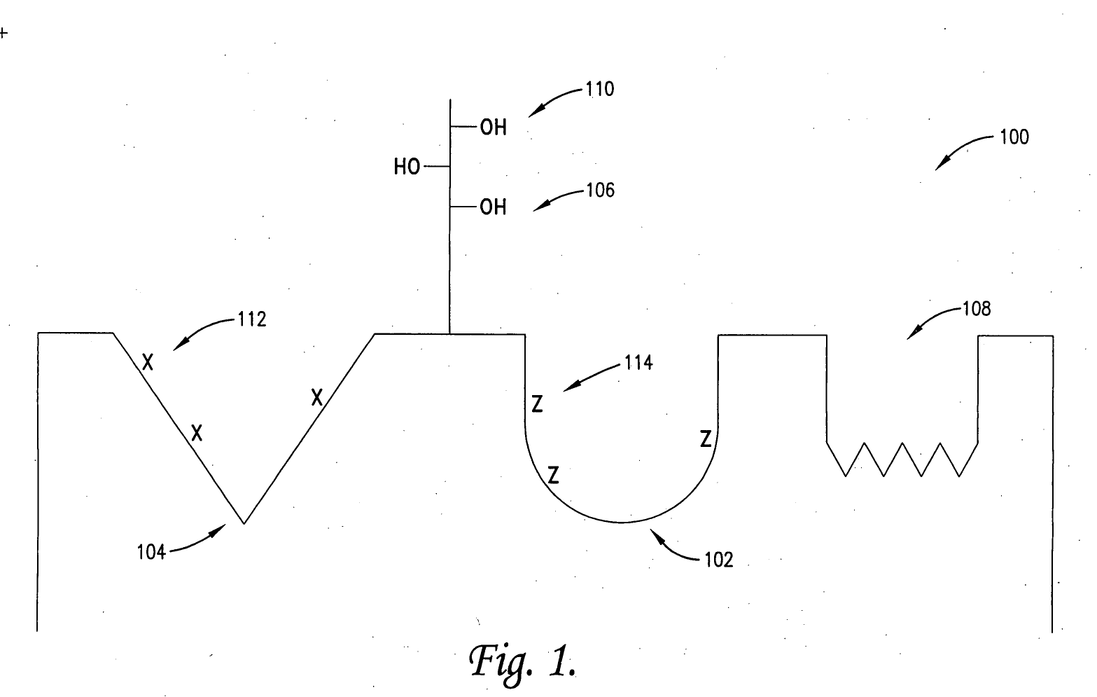

The nature of the switch control ligand/switch control pocket interaction may be understood from a consideration of schematic FIGS. 1-4. Specifically, in FIG. 1, a protein 100 is illustrated in schematic form to include an “on” switch control pocket 102, and “off” switch control pocket 104, and a switch control ligand 106. In addition, the schematically depicted protein also includes an ATP active site 108. In the exemplary protein of FIG. 1, the ligand 106 has three modifiable amino acid residues with side chain phosphorylatable OH groups 110 (typically, these amino acid residues include serine, threonine, or tyrosine). The off pocket 104 contains corresponding X conformational control residues 112 and the on pocket 102 has conformational control Z residues 114. Hence, the X and Z residues can be thought of as conformational control amino acid residues which are capable of binding with the corresponding residues forming a part of the ligand 106.

In the exemplary instance, the protein 100 will change its conformation depending upon the charge status of the OH groups 110 on ligand 106, i.e., when the OH groups are unmodified, a neutral charge is presented, but when these groups are phosphorylated a negative charge is presented.

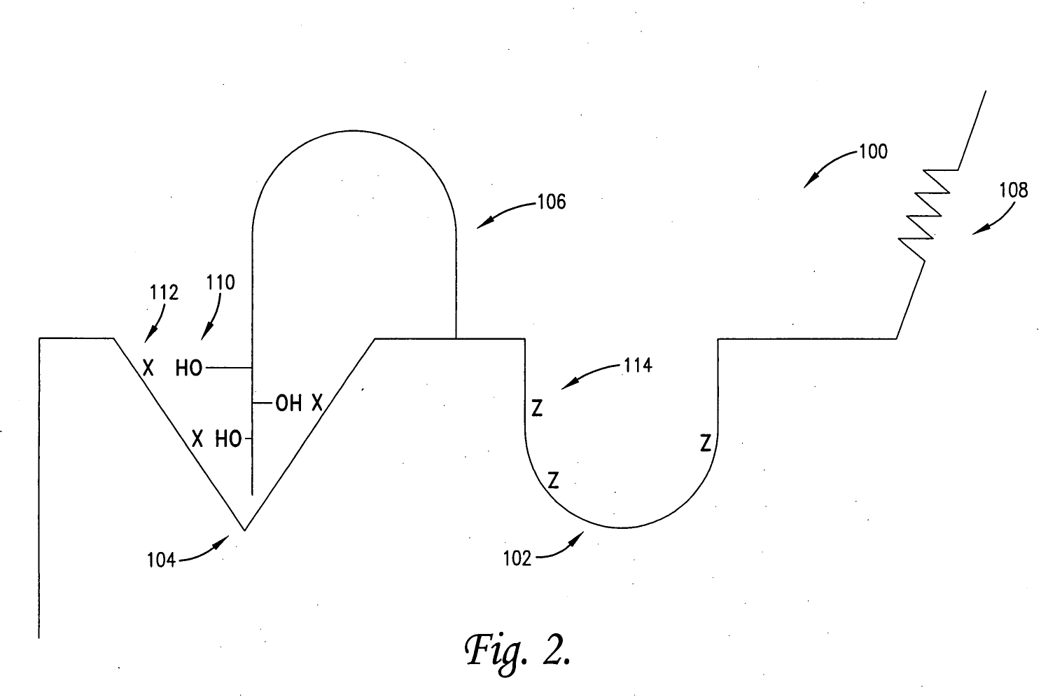

The functionality of the pockets 102, 104 and ligand 106 can be understood from a consideration of FIGS. 2-4. In FIG. 2, the ligand 106 is shown operatively interacted with the off pocket 104 such that the OH groups 110 interact with the X conformational control residues 112 forming a part of the pocket 104. Such interaction is primarily by virtue of hydrogen bonding between the OH groups 110 and the residues 112 along with additional hydrophobic, van der Waals interactions, and London dispersion forces. As seen, this ligand/pocket interaction causes the protein 100 to assume a conformation different from that seen in FIG. 1 and corresponding to the off or biologically downregulated conformation of the protein.

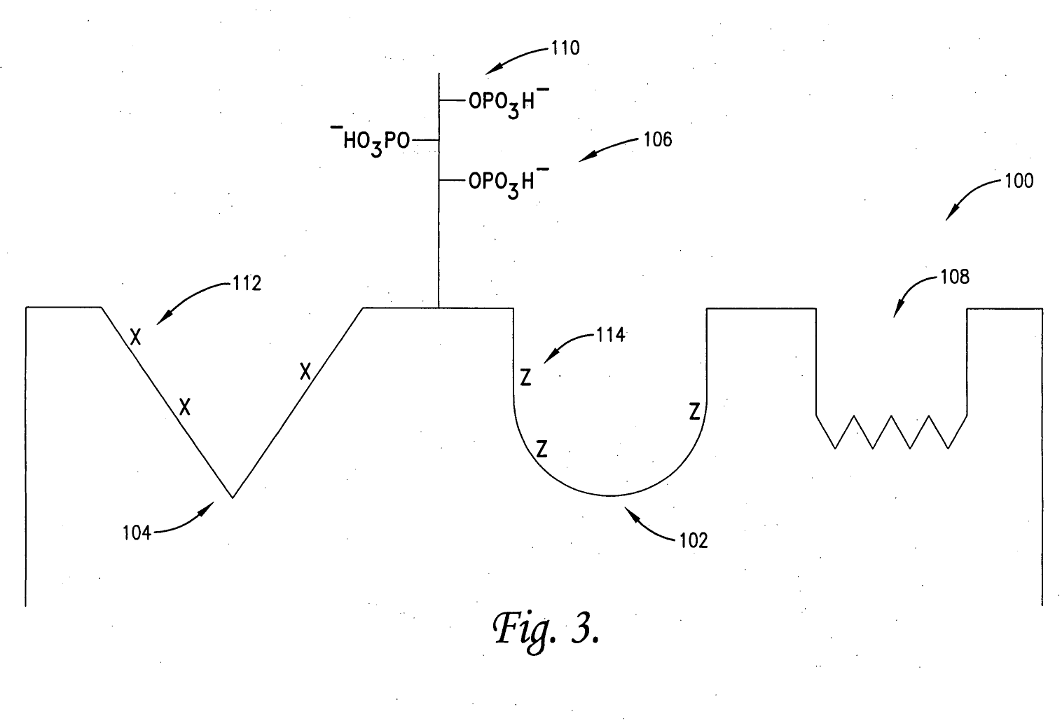

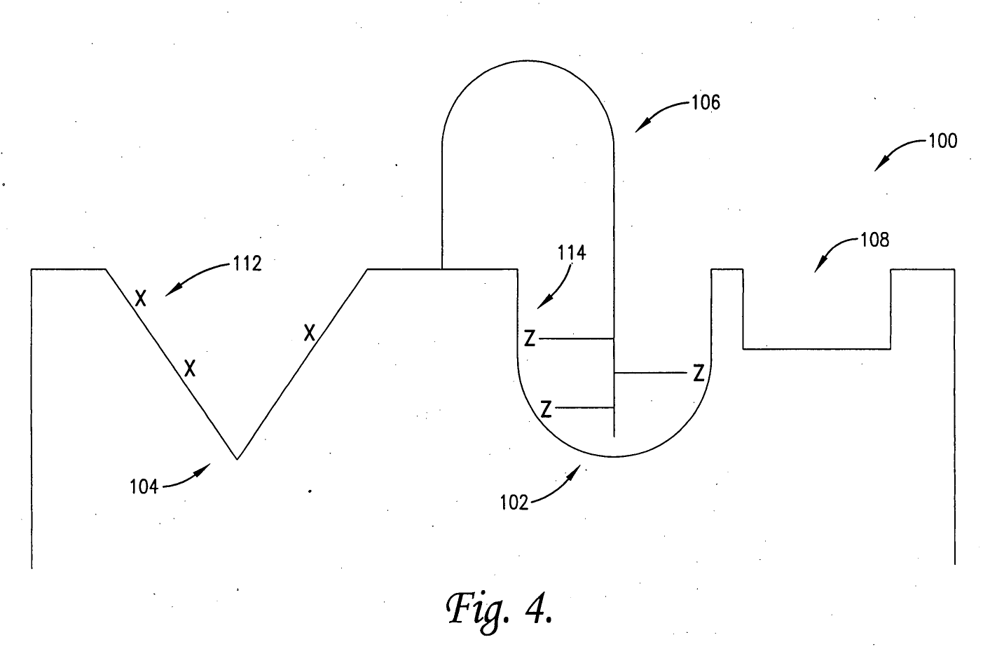

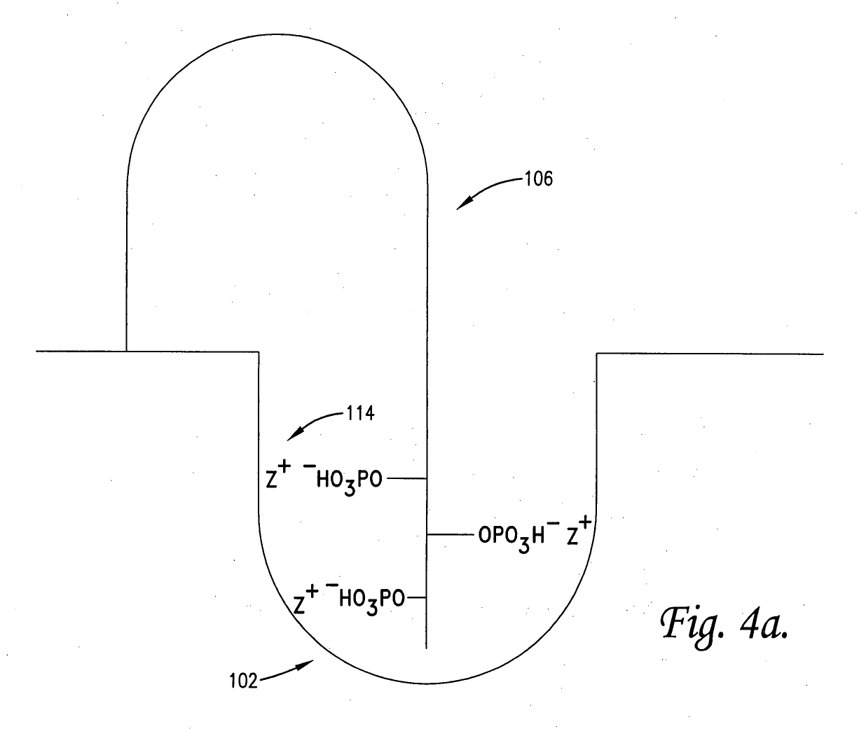

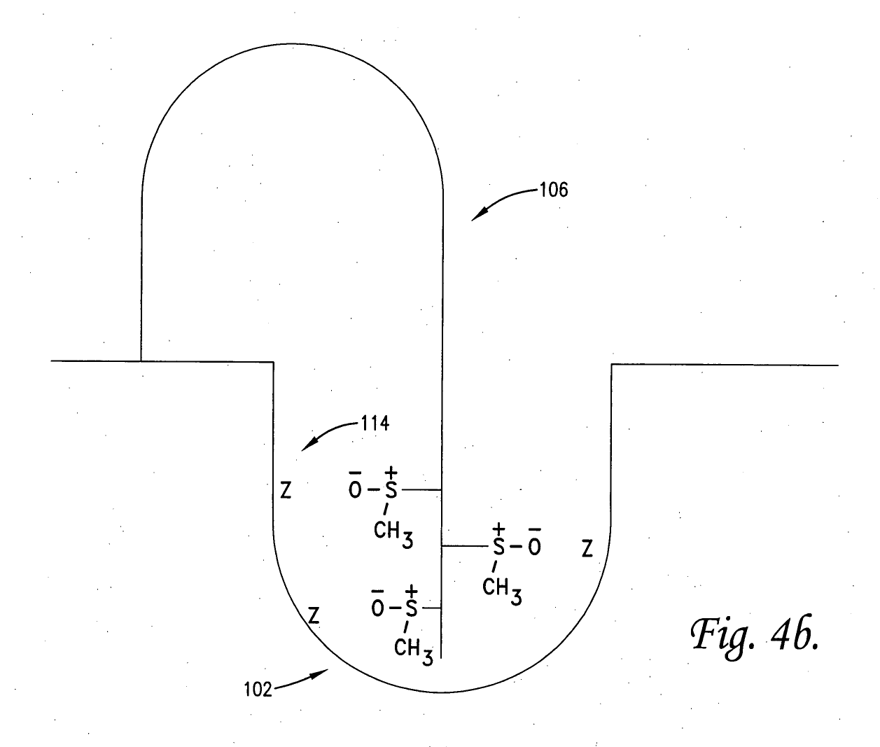

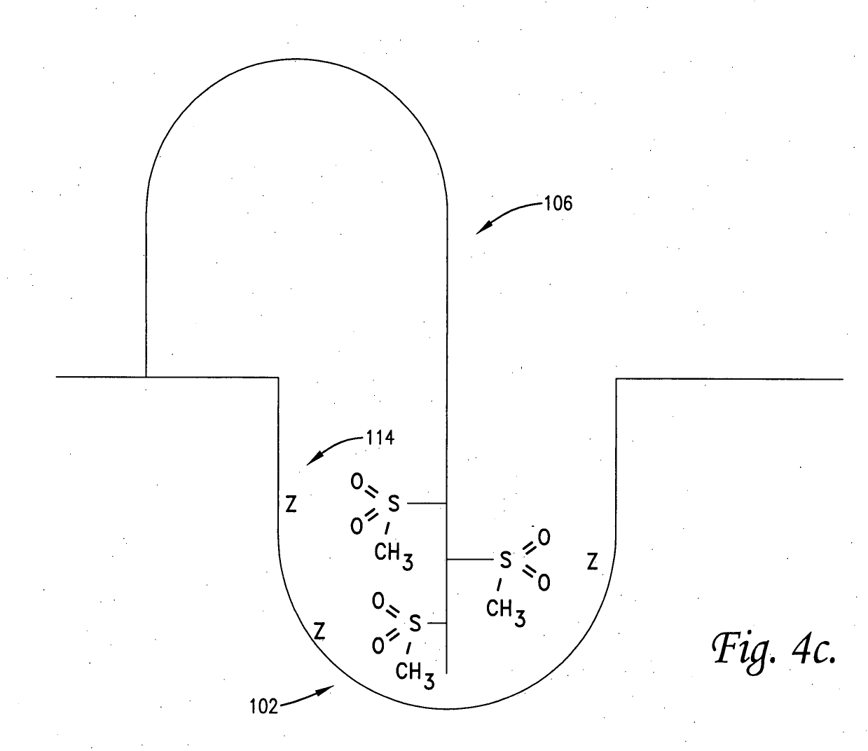

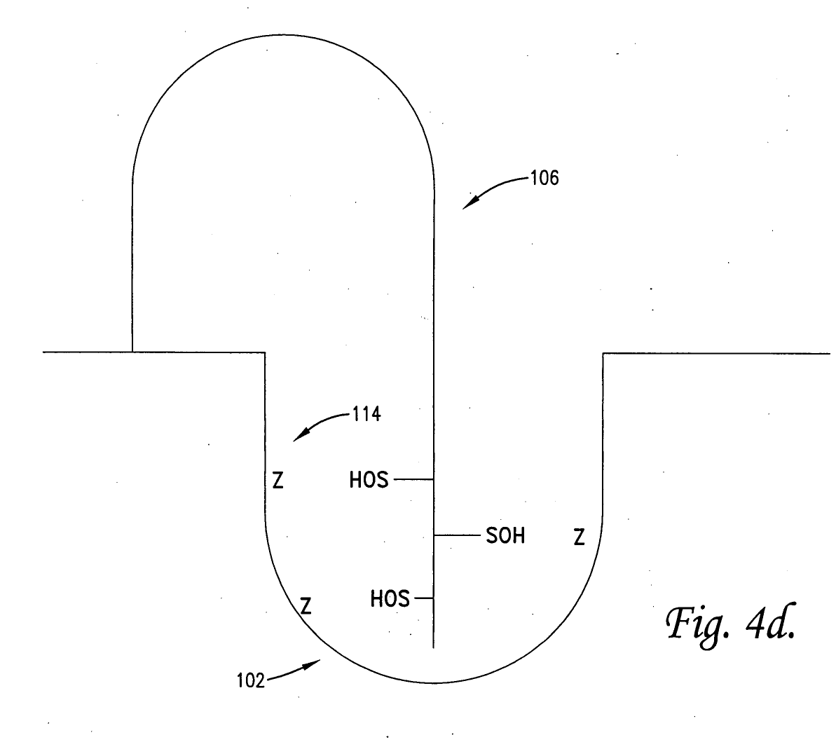

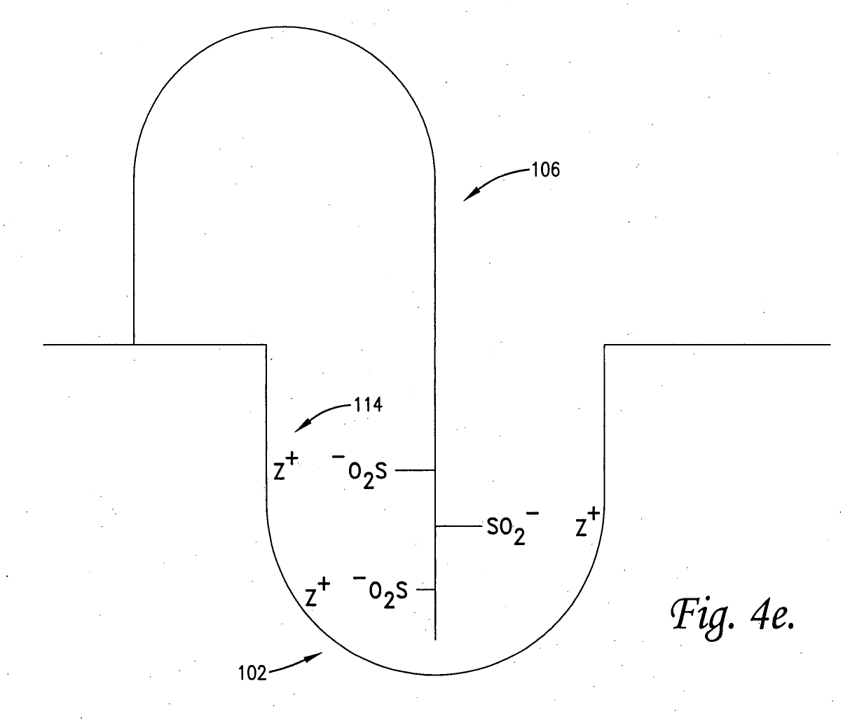

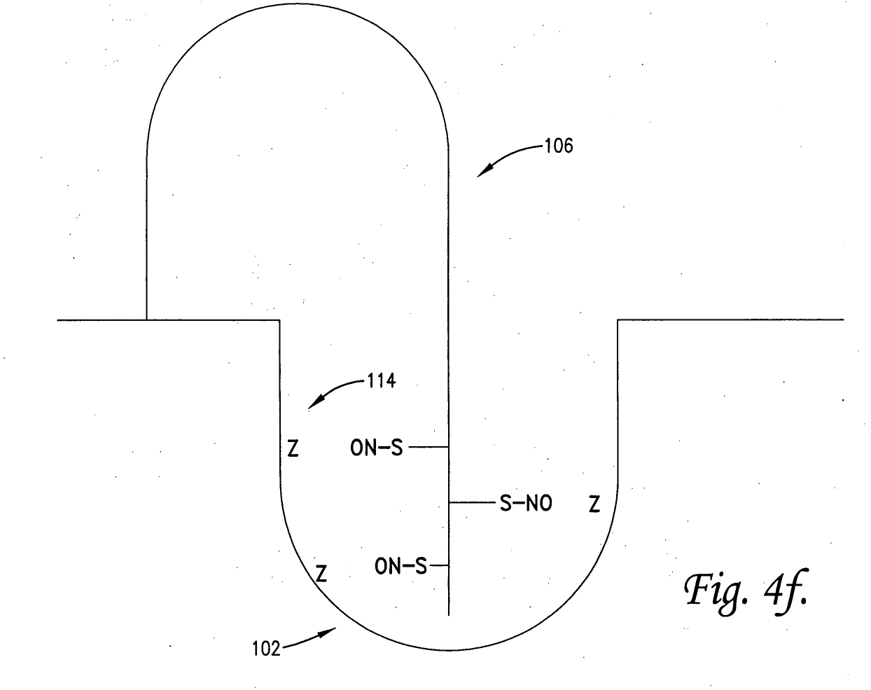

FIG. 3 illustrates the situation where the ligand 106 has shifted from the off pocket interaction conformation of FIG. 2 and the OH groups 110 have been phosphorylated, giving a negative charge to the ligand. In this condition, the ligand has a strong propensity to interact with on pocket 102, to thereby change the protein conformation to the on or biologically upregulated state (FIG. 4). FIG. 4a illustrates that the phosphorylated groups on the ligand 106 are attracted to positively charged conformational control Z residues 114 (typically including arginine or lysine residues) to achieve an ionic-like stabilizing bond between the groups 110 and the Z residues. Note that in the on conformation of FIG. 4, the protein conformation is different than the off conformation of FIG. 2, and that the ATP active site is available and the protein is functional as a kinase enzyme.

The foregoing discussion has centered upon a switch control ligand having phosphorylatable residues. However, the invention is not so limited and encompasses a variety of types of ligand modifications. For example, many protein kinases contain methionine and/or cysteine amino acid residues within the switch control ligand region; these residues are typically modified by oxidation reactions rather than phosphorylation.

Under conditions of oxidative stress, methionine residues within a switch control ligand may undergo oxidation to produce methionine sulfoxide or methionine sulfone derivatized amino acid residues. These modified methionine residues provide electronegativity to induce the switch control ligand to adopt its on or off switch state. FIG. 4b illustrates a switch control ligand wherein one or more methionine amino acid residues are modified to methionine sulfoxides, such modification inducing the switch control ligand 106 to occupy the on switch pocket 102. FIG. 4c illustrates a switch control ligand wherein one or more methionine amino acid residues are modified to methionine sulfones, such modification inducing the switch control ligand to occupy the on switch pocket 102.

The following scheme illustrates the oxidative conversion of a switch control ligand methionine residue to either a methionine sulfoxide or a methionine sulfone. The oxidative modification of methionine residues to methionine sulfone residues likely proceeds through intermediate oxidation of methionine residues to methionine sulfoxide residues.

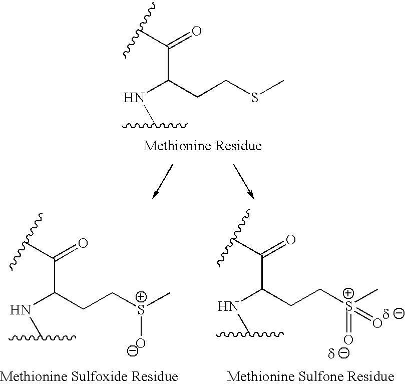

Of course, if more than one methionine residue is present within a respective switch control ligand sequence, one or more of such methionine residues may be oxidized to modulate the switch mechanism. Moreover, one or more methionine residues may be modified to a methionine sulfoxide, while another methionine residue may be concomitantly oxidized to a methionine sulfone.

Under conditions of oxidative stress, cysteine residues within the switch control ligand may undergo oxidation to produce cysteine sulfenic acids or cysteine sulfinic acid derivatized amino acid residues. These modified cysteine residues provide electronegativity or negative charge to induce the switch control ligand to adopt its on or off switch state. FIG. 4d illustrates a switch control ligand wherein one or more cysteine amino acid residues are modified to cysteine sulfenic acids, such modification inducing the switch control ligand to occupy the on switch pocket 102. FIG. 4e illustrates a switch control ligand wherein one or more cysteine amino acid residues are modified to cysteine sulfinic acids, such modification inducing the switch control ligand to occupy the on switch pocket 102.

The following scheme illustrates the oxidative conversion of a switch control ligand cysteine residue to either a cysteine sulfenic acid or a cysteine sulfinic acid. The oxidative modification of cysteine residues to cysteine sulfinic acid residues likely proceeds through intermediate oxidation of cysteine residues to cysteine sulfenic acid residues.

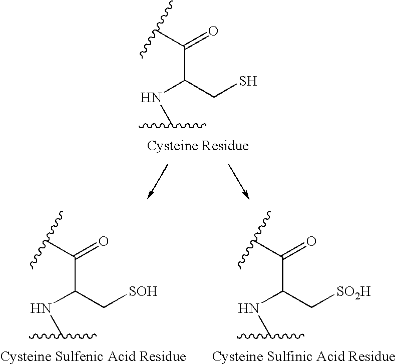

Of course, if more than one cysteine is present within a respective switch control ligand sequence, one or more of such cysteine residues may be oxidized to modulate the switch mechanism. Moreover, one cysteine residue may be modified to a cysteine sulfenic acid, while another cysteine residue may be concomitantly oxidized to a cysteine sulfinic acid.

Finally, those skilled in the art will appreciate that if a switch control ligand contains one or more cysteine and methionine residues, combinations of oxidized cysteine and oxidized methionine residues may act in concert to modify the switch control ligand and induce it to occupy an on or off switch state.

Another mechanism of transient switch control ligand modification involves S-nitrosylation of cysteine residues located within a switch control ligand sequence, in order to induce the ligand to adopt an on or off state. These S-nitrosylated cysteine residues provide electronegativity to induce the switch control ligand to adopt its on or off switch state. This may involve interactions of various types, such as transfer of the NO moieties from cysteine to an arginine Z group from pocket 102 or an arginine X group from pocket 104, or a straightforward bonding interaction. FIG. 4f illustrates a switch control ligand wherein one or more cysteine amino acid residue is modified to an S-nitrosylated cysteine residue, such modification inducing the switch control ligand to occupy the on switch pocket 102.

The following scheme illustrates the oxidative conversion of a switch control ligand cysteine residues to an S-nitrosylated cysteine residue.

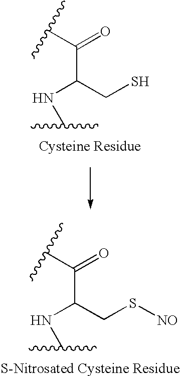

In some cases, a pair of proximal cysteine residues may be modified to form a cystine dimer, wherein two proximal cysteine residues are oxidatively bonded to each other by a disulfide bond. In these cases, one cysteine residue is transiently oxidized to a cysteine sulfenic acid or S-nitrosylated cysteine, and the second cysteine subsequently displaces the oxidized moiety on the first cysteine to form the cystine disulfide dimer. Of course, there are other mechanisms for forming disulfide cystine dimers that do not proceed through the intermediacy of cysteine sulfenic acids or S-nitrosylated cysteine residues, and these other mechanisms are encompassed herein.

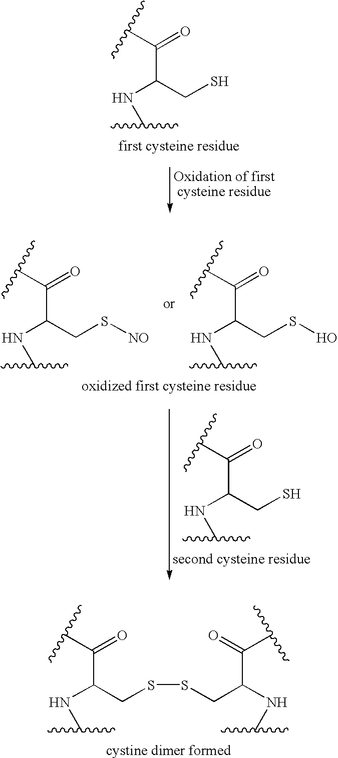

As before, it will be understood that if a switch control ligand contains one or more cysteine and methionine residues, combinations of oxidized cysteine and methionine residues may act in concert to modify the switch control ligand and induce it to occupy an on or off switch state, wherein the oxidized cysteine residues may be combinations of cysteine sulfenic acid residues, cysteine sulfinic residues, or S-nitrosylated cysteine residues. Kinases that possess a methionine as part of their switch control ligand sequence are exemplified (but not limited to) in Tables 1a and 1b. Kinases that possess a cysteine as part of their switch control ligand sequence are exemplified (but not limited to) in Tables 2a and 2b. Kinases that possess both a methionine and cysteine as part of their switch control ligand sequence are exemplified (but not limited to) in Tables 3a and 3b. Kinases that possess neither a metheonine nor a cysteine as part of their switch control ligand sequence are exemplified (but not limited to) in Table 4.

TABLE 1a
Representative human kinases that possess one or more methionines in the activation loop.

| | | |
|---|---|---|
| Kinase Name + GenBank Identifier | Activation Loop Sequence | Seq ID No. |

| | | |
|---|---|---|
| >BRAF - NM_004333; | DFGLATVKSRWSGSHQFEQLSGSILNMAPE | 41 |
| residues(DFG-->APE) = 593 to 623 (length = 30) | | |
| >CRaf - NM_002880; | DFGLATVKSRWSGSQQVEQPTGSVLWMAPE | 62 |
| residues(DFG-->APE) = 485 to 515 (length = 30) | | |
| >ABL-1 - NM_005157; | DFGLSRLMTGDTYTAHAGAKFPIKWTAPE | 63 |
| residues(DFG-->APE) = 380 to 409 (length = 29) | | |
| >FLT-3 - NM_004119; | DFGLARDIMSDSNYVVRGNARLPVKWMAPE | 64 |
| residues(DFG-->APE) = 828 to 858 (length = 30) | | |
| >HER-2 - NM_004448; | DFGLARLLDIDETEYHADGGKVPIKWMALE | 65 |
| residues(DFG-->APE) = 862 to 892 (length = 30) | | |
| >IRK-1 - NM_000208; | DFGMTRDIYETDYYRKGGKGLLPVRWHAPE | 66 |
| residues(DFG-->APE) = 1176 to 1206 (length = 30) | | |
| >KDR - NM_002253; | DFGLARDIYKDPDYVRKGDARLPLKWMAPE | 67 |
| residues(DFG-->APE) = 1045 to 1075 (length = 30) | | |
| >cKit - NM_000222; | DFGLARDIKNDSNYVVKGNARLPVKWMAPE | 68 |
| residues(DFG-->APE) = 809 to 839 (length = 30) | | |
| >cMET - NM_00245; | DFGLARDMYDKEYYSVHNKTGAKLPVKNMALE | 69 |
| residues(DFG-->APE) = 1221 to 1253 (length = 32) | | |
| >p38a - NM_001315; | DFGLARHTDDEHTGYVATRWYRAPE | 70 |
| residues(DFG-->APE) = 167 to 192 (length = 25) | | |

TABLE 1b
Other representative human kinases that possess one or more methionines in the activation loop.

| | | | |
|---|---|---|---|
| EGFR1 - NM_002350 | p36d - NM_002754 | CDK-6 - NM_001259 | ACTR-2B - NM_001106 |
| FES - NM_002005 | PDGFR-A - NM_006206 | COT - NM_005204 | ADCK-3 - NM_020247 |
| FGFR-1 - NM_000604 | PDGFR-B - NM_002609 | EGFR - NM_005228 | CDK-4 - NM_000075 |
| FGFR-3 - NM_000142 | ROS1 - NM_002944 | FAK - NM_005607 | CDK-6 - NM_001259 |
| FGFR-4 - NM_002011 | TRK-A - NM_002529 | FLT4 - NM_002020 | COT - NM_005204 |
| p38b - NM_002751 | TRK-B - NM_006180 | HER-3 - NM_001982 | EGFR - NM_005228 |
| p38g - NM_002969 | CDK-4 - NM_000075 | IGF1R - NM_000875 | FAK - NM_005607 |
| BMPR-2 - NM_001204 | FMS - NM_005211 | FLT1 - NM_002019 | FGFR2 - NM_000141 |
| BMX - NM_001721 | HPK1 - NM_007181 | HH498 - NM_015978 | GCK - NM_004579 |
| CaMKK-1 - NM_032294 | CK-1g2 - NM_001319 | CK-1g3 - NM_004384 | CRIK - NM_007174 |
| ADCK-3 - NM_020247 | CaMKK-2 - NM_006549 | CDK-10 - NM_052987 | CK-1g1 - NM_022048 |
| FLT4 - NM_002020 | HER-3 - NM_001982 | IGF1R - NM_000875 | ACTR-2B - NM_001106 |
| FER - NM_005246 | EphA4 - NM_004438 | MAP2K5 - NM_145160 | MAP2K2 - NM_030662 |
| DRAK1 - NM_004760 | DLK - NM_006301 | CRIK - NM_007174 | MAP3K1 - XM042066 |
| MAP2K1 - NM_002755 | KHS1 - NM_006575 | IRAK4 - NM_016123 | MST4 - NM_016542 |
| LZK - NM_004721 | JNK3 - NM_002753 | NEK2 - NM_002497 | MST3 - NM_003576 |
| LTK - NM_002344 | JNK1 - NM_002750 | MYO3B - NM_138995 | MST2 - NM_006281 |
| LOK - NM_005990 | ITK - NM_005546 | MYO3A - NM_017433 | MST1 - NM_006282 |
| KHS2 - NM_003618 | IRR - NM_014215 | MUSK - NM_005592 | MLK4 - NM_032435 |
| MLK3 - NM_002419 | RET - NM_020630 | SgK288 - NM_178510 | TXK - NM_003328 |
| MLK2 - NM_002446 | PKR - NM_002759 | ZC4/NRK - NM_198465 | TRKC - NM_002530 |
| MLK1 - NM_033141 | TESK2 - NM_007170 | ZC3/MINK - NM_015716 | TTK - NM_003318 |
| PEK - NM_004836 | TESK1 - NM_006285 | ZC2/TNIK - XM039796 | TLK2 - NM_006852 |
| NLK - NM_016231 | TAO3 - NM_016281 | ZC1/HGK - NM_004834 | TLK1 - NM_012290 |
| ROS - NM_002944 | TAO2 - NM_016151 | ZAK - NM_016653 | TGFbR2 - NM_003242 |
| RON - NM_002447 | TAO1 - NM_020791 | YSK1 - NM_006374 | |
| RIPK2 - NM_003821 | SLK - NM_014720 | ULK3 - NM_015518 | |

TABLE 2a
Representative human kinases that possess one or more cysteines in the activation loop.

| | | |
|---|---|---|
| Kinase Name + GenBank Identifier | Activation Loop Sequence | Seq ID No. |

| | | |
|---|---|---|
| >Aur-A - NM_003600; | DFGWSVHAPSSRRTTLCGTLDYLPPE | 71 |
| residues(DFG-->APE) = 273 to 299 (length = 26) | | |
| >FGR - NM_005248; | DFGLARLIKDDEYNPCQGSKFPIKWTAPE | 72 |
| residues(DFG-->APE) = 399 to 428 (length = 29) | | |
| >GSK-3a - NM_019884; | DFGSAKQLVRGEPNVSYICSRYYRAPE | 73 |
| residues(DFG-->APE) = 262 to 289 (length = 27) | | |
| >GSK-3b - NM_002093; | DFGSAKQLVRGEPNVSYICSRYYRAPE | 74 |
| residues(DFG-->APE) = 199 to 226 (length = 27) | | |
| >HSER - NM_004963; | DFGCNSILPPKKDLWTAPE | 75 |
| residues(DFG-->APE) = 631 to 650 (length = 19) | | |
| >HUNK - NM_014586; | DFGLSNCAGILGYSDPFSTQCGSPAYAAPE | 76 |
| residues(DFG-->APE) = 203 to 233 (length = 30) | | |
| >KSR1 - XM290793; | DFGLFGISGVVREGRRENQLKLSHDWLCYLAPE | 77 |
| residues(DFG-->APE) = 1047 to 1080 (length = 33) | | |
| >MOK - NM_014226; | DFGSCRSVYSKQPYTEYISTRWYRAPE | 78 |
| residues(DFG-->APE) = 144 to 171 (length = 27) | | |
| >MOS - NM_005372; | DFGCSEKLEDLLCFQTPSYPLGGTYTHRAPE | 79 |
| residues(DFG-->APE) = 218 to 249 (length = 31) | | |
| >SGK - NM_005627; | DFGLCKENIEHNSTTSTFCGTPEYLAPE | 80 |
| residues(DFG-->APE) = 239 to 267 (length = 28) | | |

TABLE 2b
Other representative human kinases that possess one or more cysteines in the activation loop.

| | | | |
|---|---|---|---|
| ADCK-4 - NM_024876 | KSR2 - NM_173598 | MAPKAPK3 - NM_004635 | QSK - NM_025164 |
| HIPK3 - NM_005734 | IRE1 - NM_001433 | MAPKAPK2 - NM_004759 | QIK - NM_015191 |
| HIPK1 - NM_152696 | NEK8 - NM_178170 | PKACg - NM_002732 | PRKY - NM_002760 |
| DYRK4 - NM_003845 | NEK1 - NM_01222 | PKACa - NM_002730 | PRKX - NM_005044 |
| DYRK3 - NM_003582 | MSK1 - NM_004755 | PHKg2 - NM_000294 | PLK3 - NM_004073 |
| DYRK2 - NM_006482 | MOK - NM_014226 | PHKg1 - NM_006213 | PLK1 - NM_005030 |
| DCAMKL2 - NM_152619 | MARK3 - NM_002376 | PASK - NM_015148 | PKN3 - NM_013355 |
| DCAMKL1 - NM_004734 | MARK1 - NM_018650 | RSK4 - NM_014496 | PKG2 - NM_006259 |
| PKG1 - NM_006258 | SIK - NM_173354 | TYRO3 - NM_006293 | TSSK2 - NM_053006 |
| SSTK - NM_032037 | SGK3 - NM_013257 | TSSK3 - NM_052841 | SGK2 - NM_016276 |
| SNRK - NM_017719 | | | |

TABLE 3a

Representative human kinases that possess one or more methionines and
one or more cysteines in the activation loop.

| | | |
|---|---|---|
| Kinase Name + GenBank Identifier | Activation Loop Sequence | Seq ID No. |

| | | |
|---|---|---|
| >ACK - NM_005781; | DFGLMRALPQNDDHYVMQEHRKVPFAWCAPE | 81 |
| residues(DFG-->APE) = 269 to 300 (length = 31) | | |
| >AKT-1 - NM_005163; | DFGLCKEGIKDGATMKTFCGTPEYLAPE | 82 |
| residues(DFG-->APE) = 291 to 319 (length = 28) | | |
| >Aur-B - NM_004217; | DFGWSVHAPSLRRKTMCGTLDYLPPE | 83 |
| residues(DFG-->APE) = 217 to 243 (length = 26) | | |
| >CaMK-1a - NM_003656; | DFGLSKMEDPGSVLSTACGTPGYVAPE | 84 |
| residues(DFG-->APE) = 161 to 188 (length = 27) | | |
| >CHK-1 - NM_001274; | DFGLATVFRYNNRERLLNKMCGTLPYVAPE | 85 |
| residues(DFG-->APE) = 147 to 177 (length = 30) | | |
| >Erk5 - NM_002749; | DFGMARGLCTSPAEHQYFMTEYVATRWYRAPE | 86 |
| residues(DFG-->APE) = 199 to 231 (length = 32) | | |
| >JNK2 - NM_002752; | DFGLCKEGIKDGATMKTFCGTPEYLAPE | 87 |
| residues(DFG-->APE) = 168 to 195 (length = 27) | | |
| >MAP3K2 - NM_006609; | DFGASKRLQTICLSGTGMKSVTGTPYWMSPE | 88 |
| residues(DFG-->APE) = 501 to 532 (length = 31) | | |
| >p70s6 - NM_003161; | DFGLCKESIHDGTVTHTFCGTIEYMAPE | 89 |
| residues(DFG-->APE) = 235 to 263 (length = 28) | | |
| >RSK2 - NM_004586; | DFGLSKESIDHEKKAYSFCGTVEYMAPE | 90 |
| residues(DFG-->APE) = 210 to 238 (length = 28) | | |

TABLE 3b
Representative human kinases that possess one or more methionines and one or more cysteines in the
activation loop.

| | | | |
|---|---|---|---|
| AKT-3 - NM_005465 | CaMK-1a - NM_003656 | CLIK-1L - NM_152835 | DRAK2 - NM_004226 |
| cMET - NM_00245 | CaMK-1b - NM_017275 | GCN2 - AB037759 | DMPK2 - NM_017525 |
| HER-4 - NM_005235 | CaMK-1d - NM_020397 | GAK - NM_005255 | DMPK1 - NM_004409 |
| ADCK-4 - NM_024876 | CaMK-1g - NM_020439 | Fused - NM_015690 | CYGF - NM_001522 |
| ALK - NM_004304 | CaMK-2b - NM_001220 | Erk5 - NM_002749 | CRK7 - NM_016507 |
| AMPKa-1 - NM_006251 | CaMK-4 - NM_001744 | Erk3 - NM_002748 | CLIK1L - NM_152835 |
| AMPKa-2 - NM_006252 | CDC-7 - NM_003503 | EphB6 - NM_004446 | MAP3K7 - NM_003188 |
| ANKRD-3 - NM_020639 | CDKL-5 - NM_003159 | EphB1 - NM_004441 | MAP3K6 - NM_004672 |
| ANPb - NM_000907 | ChaK-1 - NM_017672 | EphA3 - NM_005233 | MAP3K5 - NM_005923 |
| BRSK-1 - NM_032430 | CHK-2 - NM_007194 | eEF2K - NM_013302 | MAP3K4 - NM_006724 |
| BRSK-2 - NM_003957 | CLIK-1 - NM_080836 | DYRK1B - NM_004714 | MAP3K3 - NM_002401 |
| MAP2K7 - NM_145185 | IRAK1 - NM_001569 | NDR1 - NM_007271 | MNK2 - NM_017572 |
| MAP2K6 - NM_002758 | NEK5 - XM292160 | MSK2 - NM_003942 | MLK2 - NM_002446 |
| MAP2K4 - NM_003010 | NEK3 - NM_002498 | MSK2 - NM_003942 | MASTL - NM_032844 |
| MAP2K3 - NM_002756 | NEK11 - NM_024800 | MSK1 - NM_004755 | MAST4 - XM291141 |
| LATS1 - NM_004690 | NEK10 - NM_152534 | MRCKb - NM_006035 | MAST3 - XM038150 |
| IRE2 - XM370946 | NDR2 - NM_015000 | MRCKa - NM_003607 | MAST2 - NM_015112 |
| MAST1 - NM_014975 | MAPKAPK5 - NM_003668 | PKCbeta2* - X07109 | PAK2 - NM_002577 |
| MAP3K8 - NM_025052 | PKCd - NM_006254 | PAK6 - NM_020168 | PAK1 - NM_002576 |
| PKCb - NM_002738 | PKCa - NM_002737 | PAK4 - NM_005884 | p70S6Kb - NM_003952 |
| PINK1 - NM_032409 | PIM3 - NM_001001852 | PAK3 - NM_002578 | OSR1 - NM_005109 |
| PDHK4 - NM_002612 | PDHK3 - NM_005391 | NIM1 - NM_153361 | NuaK2 - NM_030952 |
| PDHK1 - NM_002610 | PAK5 - NM_020341 | NIK - NM_003954 | NuaK1 - NM_014840 |
| NEK9 - NM_033116 | ROCK1 - NM_005406 | PKN2 - NM_006256 | TESK1 - NM_006285 |
| RSKL1 - NM_012424 | RHOK - NM_002929 | PKN1 - NM_002741 | TEC - NM_003215 |
| RSK4 - NM_014496 | PYK2 - NM_004103 | PKCt - NM_006257 | TAK1 - NM_003188 |
| RSK3 - NM_002953 | PSKH2 - NM_033126 | PKCh - NM_006255 | STLK3 - NM_013233 |
| RSK1 - NM_021135 | PSKH1 - NM_006742 | PKCg - NM_002739 | STK33 - NM_030906 |
| ROCK2 - NM_004850 | PLK2 - NM_006622 | PKCe - NM_005400 | SMG1 - NM_014006 |
| SBK - XM370948 | ULK2 - NM_014683 | ULK1 - NM_003565 | TTBK2 - NM_173500 |
| TTBK1 - XM166453 | Trb1 - NM_025195 | TNK1 - NM_003985 | |

TABLE 4

Representative human kinases that possess neither methionines
nor cysteines in the activation loop.

| | | |
|---|---|---|
| Kinase Name + GenBank Identifier | Activation Loop Sequence | Seq ID No. |

| | | |
|---|---|---|
| >EPHA1 - NM_005232; | DFGLTRLLDDFDGTYETQGGKIPIRWTAPE | 91 |
| residues(DFG-->APE) = 766 to 796 (length = 30) | | |
| >ERK-1 - NM_002746; | DFGLARIADPEHDHTGFLTEYVATRWYRAPE | 92 |
| residues(DFG-->APE) = 183 to 214 (length = 31) | | |
| >HCK - NM_002110; | DFGLARVIEDNEYTAREGAKFPIKWTAPE | 93 |
| residues(DFG-->APE) = 398 to 427 (length = 29) | | |
| >JAK-1 - NM_002227; | DFGLTKAIETDKEYYTVKDDRDSPVFWYAPE | 94 |
| residues(DFG-->APE) = 1008 to 1039 (length = 31) | | |
| >Lck - NM_005356; | DFGLARLIEDNEYTAREGAKFPIKWTAPE | 95 |
| residues(DFG-->APE) = 381 to 410 (length = 29) | | |
| >LynA - NM_002350.; | DFGLARVIEDNEYTAREGAKFPIKWTAPE | 96 |
| residues(DFG-->APE) = 384 to 413 (length = 29) | | |
| >SRC - NM_005417; | DFGLARLIEDNEYTARQGAKFPIKWTAPE | 97 |
| residues(DFG-->APE) = 406 to 435 (length = 29) | | |
| >SYK - NM_003177; | DFGLSKALRADENYYKAQTHGKWPVKWYAPE | 98 |
| residues(DFG-->APE) = 511 to 542 (length = 31) | | |
| >YES - NM_005433; | DFGLARLIEDNEYTARQGAKFPIKWTAPE | 99 |
| residues(DFG-->APE) = 413 to 442 (length = 29) | | |
| >ZAP-70 - NM_001079; | DFGLSKALGADDSYYTARSAGKWPLKWYAPE | 100 |
| residues(DFG-->APE) = 478 to 509 (length = 31) | | |

Yet another type of modification of amino acid residues in the switch control ligand 106 involves the genomic mutation of a wild-type amino acid residue which does not function to mediate a change in conformational state of the protein, to a mutated amino acid residue which does function to mediate a change in conformational state of the protein. Such a mutated residue is of the type referred to as a modified amino acid of the switch ligand sequence which is permanent. By way of example, the following scheme depicts wild-type Braf kinase, wherein the valine amino acid residue 599, which is not a modified amino acid residue of the ligand sequence, is mutated to a glutamic acid residue 599, which is a permanently modified amino acid residue (compared to wild-type residue valine 599) that triggers the switch mechanism in Braf kinase.

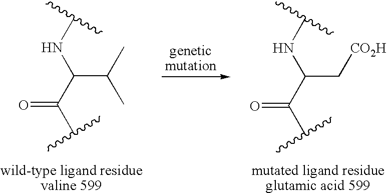

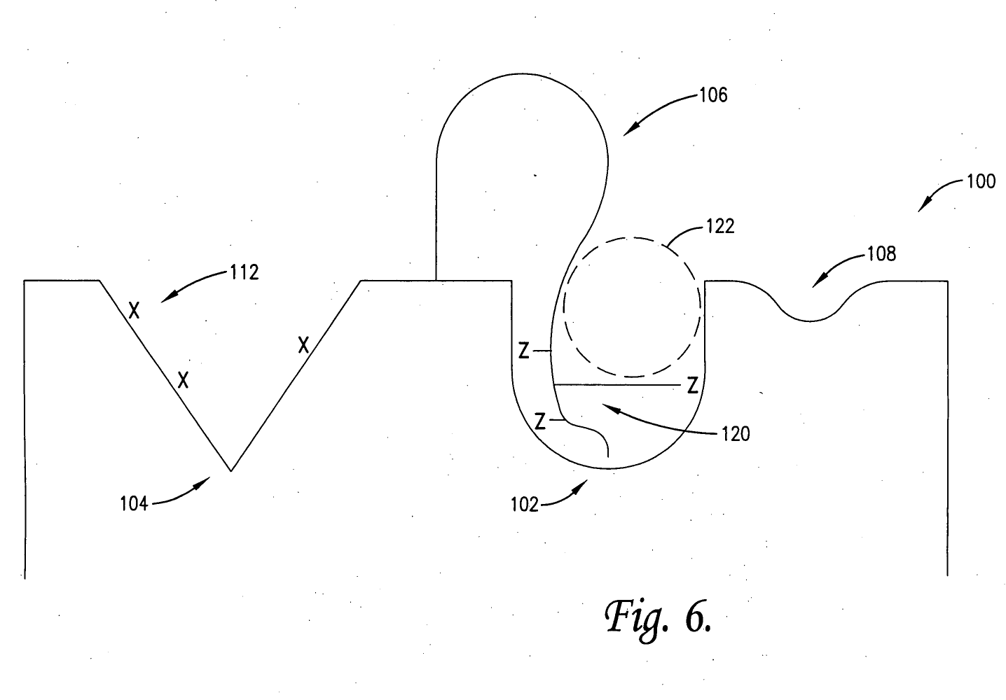

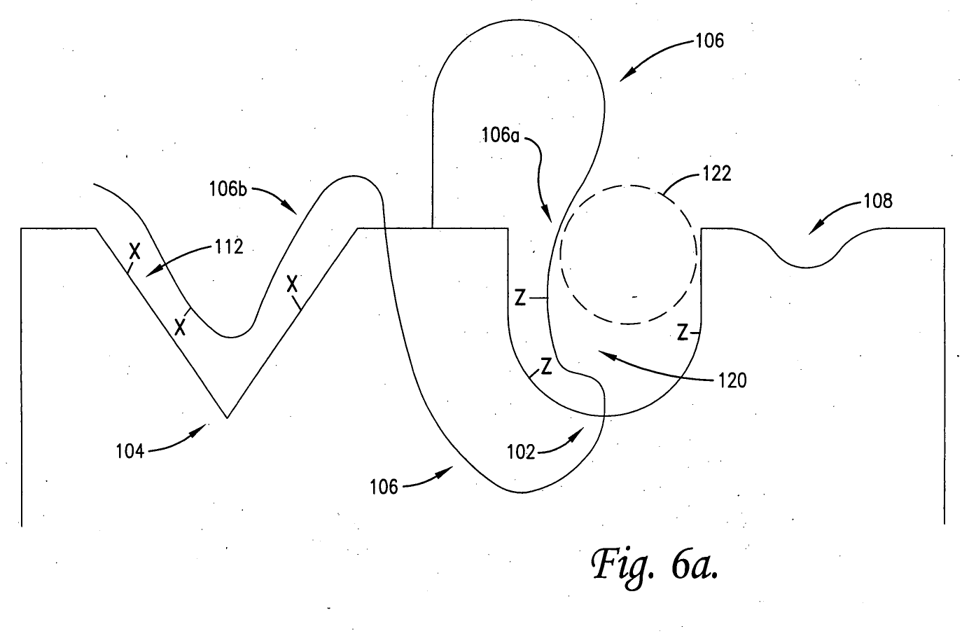

FIGS. 1-4 illustrate a simple situation where the protein exhibits discrete pockets 102 and 104 and ligand 106. However, in many cases a more complex switch control pocket pattern is observed. FIG. 6 illustrates a situation where an appropriate pocket for small molecule interaction is formed from amino acid residues taken both from ligand 106 and, for example, from pocket 102. This is termed a “composite switch control pocket” made up of residues from both the ligand 106 and a switch control pocket, and is referred to by the numeral 120. A small molecule 122 is illustrated which interacts with the pocket 120 for protein modulation purposes. Of course, the small molecule 122 binds with some or all of the conformational control residues Z of the composite pocket.

FIG. 6a is a schematic representation of a naturally occurring mammalian protein kinase in a binding relationship with a small molecule switch control inhibitor wherein the small molecule binds to the conformational control Z residues on composite switch control pocket 120 of the protein kinase. The on composite switch control pocket 120 is made up of amino acid residues taken from the on switch control pocket 102 and amino acid residues taken from the N-terminal region 106a of the switch control ligand 106. The small molecule makes binding contact with Z groups of switch control pocket 102. The inhibitor also optionally makes contact with Z groups taken from the N-terminal region of the switch control ligand 106a. Other amino acids taken from pocket 102 and the N-terminal region 106a may contribute to the composite switch control pocket. Upon binding of the small molecule to this on composite switch control pocket, the C-terminal region 106b of the switch control ligand 106 is displaced into the off switch control pocket 104. The concomitant binding of the small molecule into the on composite switch control pocket and displacement of the C-terminal region 106b into the off switch control pocket 104 functionally down-regulates the biological activity of the protein kinase. Specifically, 1) the ATP cofactor pocket is occluded by one or more amino acid residues of the N-terminal region 106a; 2) the bulk of the switch control ligand 106 occludes the protein substrate binding pocket of the protein kinase; 3) the catalytic amino acid residues including the aspartic acid from the DFG motif of 106a and combinations of the histidine, aspartic acid, and asparagine amino acids from the catalytic loop are induced to assume a catalytically downregulated conformation. Additionally, binding of the small molecule switch control inhibitor to the protein kinase induces a change in the protein conformation which modulates domains involved in dimerization or oligomerization, cell-trafficking, or participation in signaling complexes with other proteins.

In other cases it has been found that the conformational control Z or X residues of on and off pockets respectively can bind with each other in certain protein conformations, e.g., in the X-ray co-crystal structure of the switch control inhibitor of Example 29 with p38-alpha kinase, the inhibitor makes contact (binds) with X conformational control residue tyrosine-35. Tyrosine-35 also binds to arginine-67 which is a Z group from the on control pocket. In this off conformational state of p38-alpha kinase, the Z conformational control residue arginine-67 forms a stabilizing interaction with the X conformational control residue tyrosine-35.

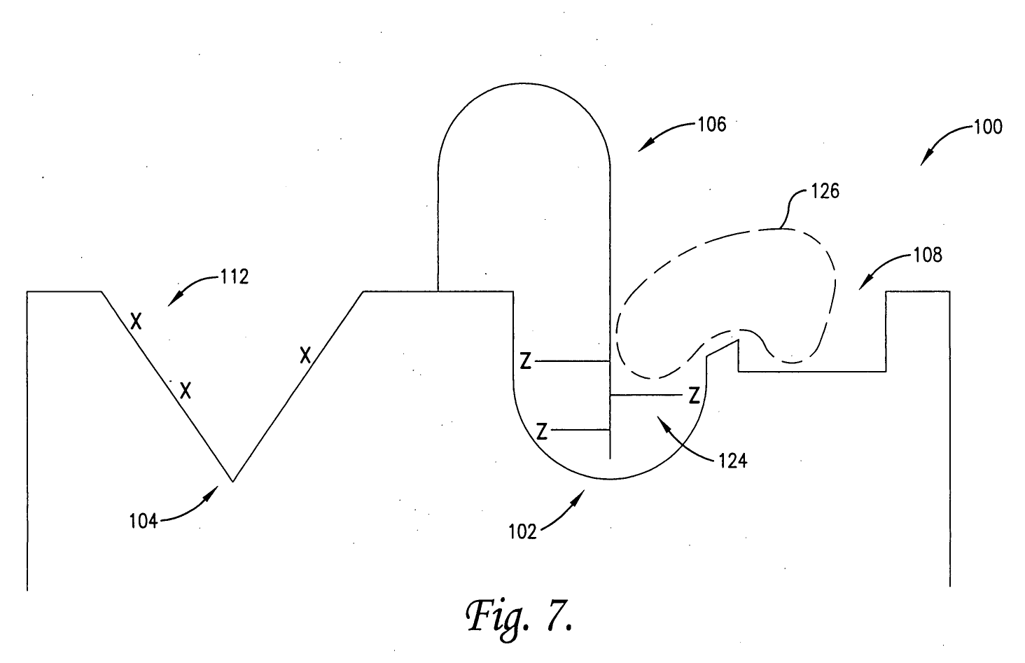

Another more complex switch pocket is depicted in FIG. 7 wherein the pocket includes residues from on pocket 102, and ATP site 108 to create what is termed a “combined switch control pocket.” Such a combined pocket is referred to as numeral 124 and may also include residues from ligand 106. An appropriate small molecule 126 is illustrated with pocket 124 for protein modulation purposes.

It will thus be appreciated that while in the simple pocket situation of FIGS. 1-4, the small molecule will interact with the simple pocket 102 or 104, in the more complex situations of FIGS. 6 and 7 the interactive pockets are in the regions of the pockets 120 or 124. Thus, broadly, the small molecules interact “at the region” of the respective switch control pocket.

The proteins useful in the invention can also be thought of as having first, second and third respective series of amino acid residues therein. The first series of residues forms a part of the ligand 106, and the residues of the first series are individually modifiable in vivo (either transiently or substantially permanently) between two respective states, usually corresponding with two different protein conformations. This first series of amino acid residues is capable of binding with the second series of residues when the first series is in one of its states, and alternately, the first series binds with the third series of residues when the first series is in the other of its states. Thus, the second and third series of residues can be analogized with the Z and X residues described previously.

The modifiable residues of a switch control ligand sequence (i.e. the first series) can be modified collectively or independently of each other. Specifically residues from the first series can be all modified substantially simultaneously between the two respective states, or in other cases, certain other residues of the first series may be modifiable separately from other residues of the first series. The residues of the first series are modifiable through a variety of mechanisms, e.g., phosphorylation, sulfation, fatty acid acylation, glycosylation, prenylation, carboxylation, nitrosylation, cystinylation, or oxidation.

In the context of protein-modulator adducts, the modulator molecule is bound to at least one of the amino acid residues of the first, second or third series. That is to say, the modulator molecule can be bound to one or more of the first (ligand) series of residues, and/or with one or more of the residues of the second and third series. Such binding can be in the nature of non-covalent reversible bonding, e.g. hydrogen bonding, hydrophobic bonding, electrostatic bonding, ionic bonding, van der Waals interactions, or London dispersion forces. Additionally, in certain instances, the bonding may involve chemical modification of an amino acid residue by removal of a residue moiety, which generally results in a change in state, for example, an amino acid residue of the first series may be modifiable by nitrosylation thereof, and a modulator molecule may remove a nitrosyl group from the residue during the binding sequence. The following scheme depicts a small molecule modulator R—SH removing a nitrosyl group from a ligand (first series) S-nitrosyl cysteine amino acid residue.

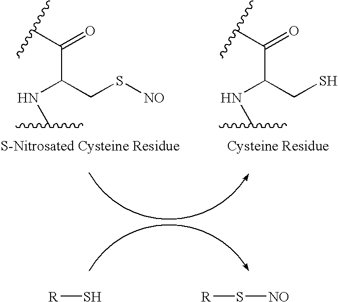

In another example, the residue of the first series may be modifiable by oxidation thereof, and the small molecule modulator may add an oxygen radical during the binding sequence. The following scheme illustrates this phenomenon wherein the small molecule modulator in an oxidized state comes in contact with a methionine amino acid residue of a ligand sequence (i.e. first series) and chemically modifies said methionine residue to an oxidized state. In this oxido-reduction mechanism the small molecule modulator gets reduced in the process of oxidizing the methionine amino acid residue.

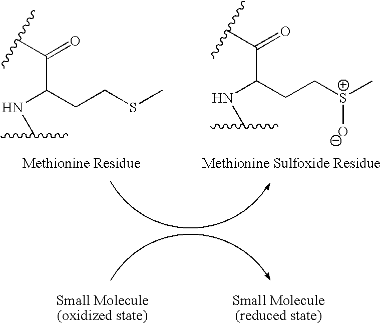

In most instances, a modulator molecule will bind with one or more residues of the second and third series (or Z or X residues described above). This situation can be exemplified through actual protein-modulator adducts.

For example, where the protein is p38-alpha kinase, an effective modulator molecule will bind to arginine 70 and/or arginine 67, which are Z residues (second series); preferably, the molecule binds with the guanidinyl moieties of either or both of these arginines. Additionally, the molecule may further bind with at least certain residues selected from the group consisting of glutamic acid 71, leucine 74, methionine 78, valine 83, isoleucine 141, histidine 148, phenylalanine 169, lysine 53, isoleucine 84, leucine 104, and threonine 106. In an alternate case, a useful modulator would bind with tyrosine 35 which is an X residue (third series), and in such a case, there may be further binding with arginine 70 and/or arginine 67, and at least certain residues selected from the group consisting of glutamic acid 71, leucine 74, methionine 78, valine 83, isoleucine 141, histidine 148, phenylalanine 169, lysine 53, isoleucine 84, leucine 104, and threonine 106.

Where the protein is a wild-type Braf kinase, a useful modulator molecule will bind with one or more of asparagine 499, lysine 600, and arginine 602, which are Z residues (second series), with possible further binding with at least certain residues selected from the group consisting of alanine 496, valine 599, glutamic acid 500, phenylalanine 594, lysine 482, leucine 513, isoleucine 526, threonine 528, valine 503, leucine 504, threonine 507, isoleucine 512, leucine 566, and histidine 573.

Similarly, where the protein is oncogenic V599E Braf kinase, a useful modulator molecule will bind with one or more of asparagine 499, lysine 600, and arginine 602, which are Z residues (second series). A useful modulator may also bind to the mutated amino acid residue glutamic acid 599 (the modified amino acid of the first series). The modulator may further bind with at least certain residues selected from the group consisting of alanine 496, glutamic acid 500, phenylalanine 594, lysine 482, leucine 513, isoleucine 526, threonine 528, valine 503, leucine 504, threonine 507, isoleucine 512, leucine 566, and histidine 573.

Wild-type c-Abl kinase or oncogenic Bcr-Abl kinase can bind with a modulator in accordance with the invention at arginine 405 (a Z residue) and/or glutamic acid 301 (an X residue). Further binding between the protein and the modulator molecule would possibly be with glutamic acid 305, phenylalanine 401, lysine 290, valine 318, isoleucine 332, threonine 334, valine 308, isoleucine 312, leucine 317, leucine 373, and histidine 380.

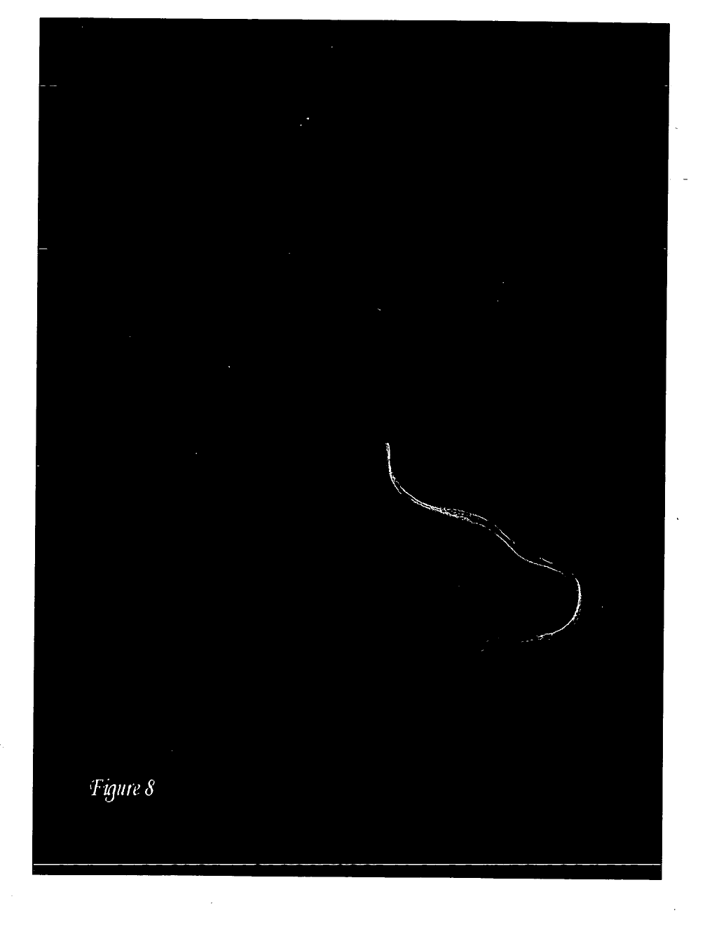

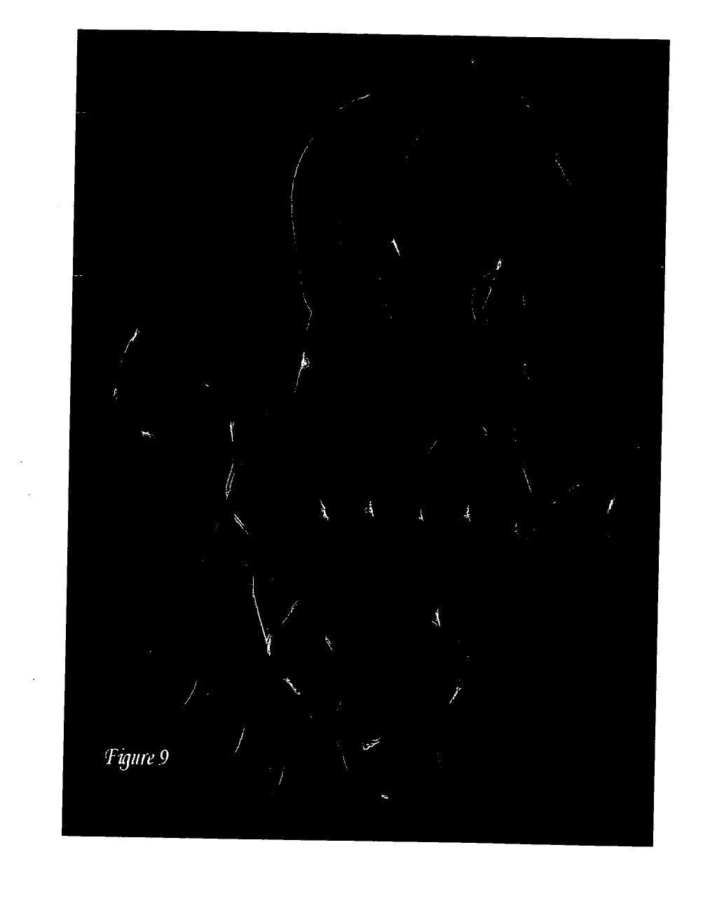

FIGS. 8 and 9 are ribbon diagrams derived from X-ray crystallography analysis of the insulin receptor kinase domain protein, where FIG. 8 illustrates the protein in its on or biologically upregulated conformation, shown in blue. In this photograph, the yellow-colored strand is the switch control ligand sequence, whereas the magenta portions represent key residues forming the complemental on-switch control pocket which interacts with the ligand sequence to maintain the protein in the biologically upregulated conformation. FIG. 9 on the other hand depicts the protein in its off or biologically downregulated conformation, shown in simulated brass color. In this diagram, the switch control sequence is again depicted in yellow and key residues of the off-switch control pocket are illustrated in green. Again, the interaction between the switch control ligand and the off-switch control pocket maintains the protein in the depicted biologically downregulated conformation.

Referring again to the schematic depictions, the FIG. 8 diagram corresponds to FIG. 4 wherein the ligand 106 interacts with on pocket 102. Likewise, FIG. 9 corresponds to FIG. 2 wherein ligand 106 interacts with pocket 104.

Those skilled in the art will appreciate that a given protein will “switch” over time between the upregulated and downregulated conformations based upon the modification of ligand 106, wherein a change in the status of the modifiable state of ligand 106 tends to shift the protein to the on pocket conformation, or tends to shift the protein to the off pocket conformation. Thus, the conformation change effected by the switch control ligand/switch control pocket interaction is dynamic in nature and is ultimately governed by intracellular conditions.

It will also be understood that abnormalities in protein conformation can lead to or exacerbate diseases. For example, if a given protein untowardly remains in the off or biologically downregulated conformation, metabolic processes requiring the active protein will be prevented, retarded or unwanted side reactions may occur. Similarly, if a protein untowardly remains in the on or biologically upregulated conformation, the metabolic process may be unduly promoted which can also result in serious health problems.

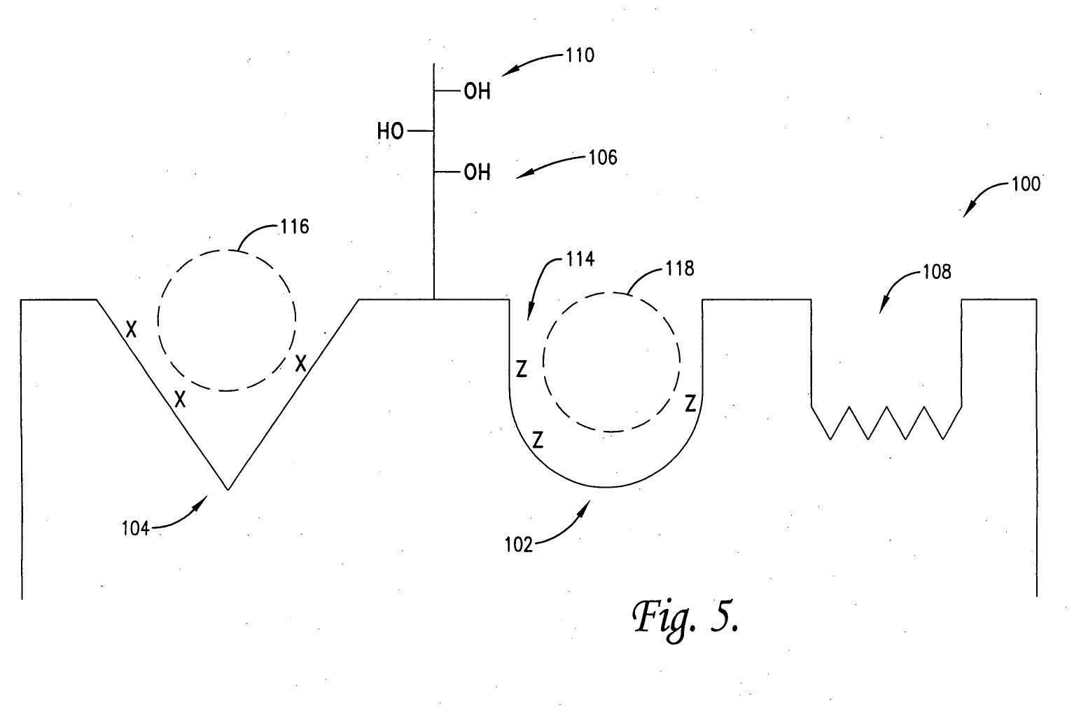

However, it has been found that small molecule compounds can be developed which will modulate protein activity so as to duplicate or approach normal in vivo protein activity. Referring to FIG. 5, it will be seen that a small molecule 116 may interact with off pocket 104 so as to inhibit ligand 106 from interacting with the pocket 104. In this simplified hypothetical, the protein 100 would then have a greater propensity to remain in the on or biologically upregulated conformation. As an alternative, a small molecule 118 is shown interacting with on pocket 102 so as to inhibit ligand 106 from interaction with the pocket 102. Under this simplified scheme, this would result in a greater propensity for the ligand 106 to interact with off pocket 104, thereby causing the protein to move to its off or biologically downregulated conformation.

Hence, analysis of proteins to ascertain the location and sequences of interacting switch control ligands and switch control pockets, together with an understanding of how these interact to switch the protein between biologically upregulated and down-regulated conformations, provides a powerful tool which can be used in the design and development of small molecule compounds which can modulate protein activity.

Broadly speaking, the method of identifying molecules which interact with specific naturally occurring proteins in order to modulate protein activity involves first identifying a switch control ligand forming a part of the protein, and a switch control pocket also forming a part of the protein and which interacts with the ligand. The ligand and pocket cooperatively interact to regulate the conformation and biological activity of the protein, such that the protein will assume a first conformation and a corresponding first biological activity upon the ligand-pocket interaction, and will assume a second, different conformation and biological activity in the absence of the ligand-pocket interaction.

In the next step, respective samples of the protein in the first and second conformations thereof are provided, and these protein samples are used in screening assays of candidate small molecules. Such screening broadly involves contacting the candidate molecules with at least one of the samples, and identifying which of the small molecules bind with the protein at the region of the identified switch control pocket.

The method of the invention is applicable to a wide variety of naturally occurring mammalian (e.g., human) proteins, which may be wild type consensus proteins, disease polymorphs, disease fusion proteins and/or artificially engineered variant proteins. Classes of applicable proteins would include enzymes, receptors, and signaling proteins; more particularly, the kinases, phosphatases, phosphodiesterases, proteases, sulfotranferases, sulfatases, transcription factors, nuclear hormone receptors, g-protein coupled receptors, g-proteins, gtp-ases, hormones, polymerases, and other proteins containing nucleotide regulatory sites. In most instances, proteins of interest would have a molecular weight of at least 15 kDa, and more usually above about 30 kDa.

In the course of the method of the invention, a number of techniques may be used to identify switch control ligand sequence(s) and switch control pocket(s) and to determine the upregulation or downregulation effects of candidate small molecule modulators. Broadly speaking, these methods comprise analysis of bioinformatics, X-ray crystallography, nuclear magnetic resonance spectroscopy (NMR), circular dichroism (CD), and affinity based screening. In addition, entirely conventional techniques such as site directed mutagenesis and standard biochemical experiments may also be of assistance.

Bioinformatic analysis permits identification of relevant ligands and pockets without the need for experimentation. For example, relevant protein data can be in some cases determined strictly through use of available databases such as PUBMED. Thus, an initial step may be a PUBMED inquiry regarding known structures of a protein of interest, which contains sequence information. Next, BLAST searches may be conducted, in order to ascertain other sequences containing a selected minimum stringency (e.g., at least 60% homology). This may reveal point mutations or polymorphisms of interest, as well as abnormal fusion proteins, all of which may be causative of disease; these may also provide insights into the identification of functional or dysfunctional switch control ligand sequences and/or pockets causative of disease. A specific example of such bioinformatic analysis is set forth in Example A below.

X-ray crystallography techniques first require protein expression affording purified proteins. Whole gene synthesis technology may be used to chemically synthesize protein genes optimized for the particular expression systems used. Conventional technology can be employed to rapidly synthesize any gene from synthetic oligonucleotides. Software (Gene Builder™) and associated molecular biology methods allow any gene to be synthesized. Whole gene synthesis is advantageous over traditional cloning methods because the codon optimized version of the gene can be rapidly synthesized for optimal expression. In addition, complex mutations (e.g. combining many different mutations) can be made in one step instead of sequentially. Strategic placement of restriction sites facilitates the rapid addition of mutations as needed. This technology therefore allows many more gene constructs to be created in a shorter amount of time. Protein sequence selection is determined using a combination of phylogenetic analyses, molecular modeling and structural predictions, known expression, functional screening data, and reported literature data to develop a strategy for protein production. Expression constructs can be made using commercially available vectors to express the proteins in baculovirus-infected insect cells. E. coli expression systems may be used for production of other proteins. The genes may be modified by adding affinity tags. The genes may also be modified by creating deletions, point mutations, and protein fusions to improve expression, aid purification and facilitate crystallization.

Protein Purification: Total cell paste from expression experiments may be disrupted by nitrogen cavitation, French press, or microfluidization which ever proves to be the most effective for releasing soluble protein. The extracts are subjected to parallel protein purification using the a robotic device that simultaneously runs multiple columns (including Glu-mAb, metal chelate, Q-seph, S-Seph, Phenyl-Seph, and Cibacron Blue) in parallel under standard procedures and the fractions are analyzed by SDS-PAGE. This information is combined with the published purification protocols to rapidly develop purification protocols. Once purified, the protein is subjected to a number of biophysical assays (Dynamic Light Scattering, UV absorption, MALDI-ToF, analytical gel filtration etc).

Crystal Growth and X-ray Diffraction Quality Analysis: Sparse matrix and focused crystallization screens are set up with and without ligands at 2 or more temperatures. Crystals obtained without ligands (apo-crystals) are used for ligand soaking experiments. Crystal growth conditions are optimized for protein-crystals based on initial results. Once suitable protein-crystals have been obtained, they are screened to determine their diffraction quality under various cryo-preservation conditions on an R-AXIS IV imaging plate system and an X-STREAM cryostat Protein-crystals of sufficient diffraction quality are used for X-ray diffraction data collection, or are stored in liquid nitrogen and saved for subsequent data collection at a synchrotron X-ray radiation source. The diffraction limits of protein-crystals are determined by taking at least two diffraction images at phi spindle settings 90° apart. The phi spindle is oscillated 1° during diffraction image collection. Both images are processed by the HKL-2000 suite of X-ray data analysis and reduction software. The diffraction resolution of the protein-crystals are accepted as the higher resolution limit of the resolution shell in which 50% or more of the indexed reflections have an intensity of 1 sigma or greater.

X-ray Diffraction Data Collection: If the protein-crystals are found to diffract to 3.0 Å or a better on the R-AXIS IV system or at a synchrotron, then a complete data set is collected at a synchrotron. A complete data set is defined as having at least 90% of all reflections in the highest resolution shell having been collected. The X-ray diffraction data are processed (reduced to unique reflections and intensities) using the HKL-2000 suite of X-ray diffraction data processing software.

Structure Determination: The structures of the proteins are determined by molecular replacement (MR) using one or more protein search models. This MR method uses the protein coordinate sets available in the Protein Data Bank (PDB). If necessary, the structure determination is facilitated by multiple isomorphous replacement (MIR) with heavy atoms and/or multi-wavelength anomalous diffraction (MAD) methods. MAD synchrotron data sets are collected for heavy atom soaked crystals if EXAFS scans of the crystals (after having been washed in mother liquor or cryoprotectant without heavy atom) reveal the appropriate heavy atom signal. Analysis of the heavy atom data sets for derivatization is completed using the CCP4 crystallographic suite of computational programs. Heavy atom sites are identified by (|FPH|−|FP|)² difference Patterson and the (|F+|−|F−|)₂ anomalous difference Patterson map.

High field nuclear magnetic resonance MR) spectroscopic methods can also be utilized to identify switch control ligand sequences and pockets. NMR studies have been reported to elucidate the structures of proteins and in particular protein kinases. (Wuthrich, K; “NMR of Proteins and Nucleic Acids” Wiley-Interscience: 1986; Evans, J. N. S., Biomolecular Nmr Spectroscopy, Oxford University Press: 1995; Cavanagh, J.; et al., N. Protein Nmr Spectroscopy: Principals and Practice, Academic Press: 1996.; Krishna, N. R.; Berliner, L. J. Protein Nmr for the Millennium (Biological Magnetic Resonance, 20), Plenum Pub Corp: 2003.

Over the last 20 years, NMR has evolved into a powerful technique to probe protein structures, to probe the interaction of proteins with other biomolecules and to expose the details of small-molecule-protein interactions. NMR methods are complementary to X-ray crystallographic methods, and the combination of the two techniques provides a powerful strategy to reveal the nature of protein/small molecule interactions. A particularly advantageous NMR technique involves the preparation of ¹⁵N and/or ¹³C labeled protein and analyzing chemical shift perturbations which occur upon conformational changes of the protein effected by interaction of the protein's switch control ligand sequence with its respective switch control pocket or interaction of a small molecule modulator with a switch control pocket region.

NMR chemical shift perturbations studies provide a powerful method to probe the dynamics of docking of switch control inhibitors into the switch control region of kinases. This approach is well described in the primary literature, and is accepted as a useful tool for probing the interaction of small molecules with protein substrates (See: Pellecchia M, Sem D S, Wuthrich K. “NMR in drug discovery”, Nat Rev Drug Discov. 2002 March; 1(3):211-9 and references therein). The principle follows that which has been previously described in the primary literature, and relies on changes in the local magnetic environment of protein, induced by the ligand, to cause a change in the chemical shifts of the ¹H, ¹³C and ¹⁵N resonances of the protein backbone and side chains that directly interact with the ligand. It is understood that these studies require ¹⁵N and ¹⁵N/¹³C labeled protein, either uniformly labeled in the cases of small proteins (<20 kD) or specifically labeled proteins in the case of larger proteins (>20 kD).

In the case of a protein substrate whose molecular weight is <20 kD, one skilled in the art assigns all the ¹H, ¹³C and ¹⁵N resonances of the uniformly isotopically labeled apo-protein of interest (such as a kinase) using modern NMR pulse sequences including (but not limited to) COSY, NOESY, TOCSY/HOHAHA, HSQC, HNCA, HNCOCA, and NCOCACB (Wuthrich K. “Determination of three-dimensional protein structures in solution by nuclear magnetic resonance: an overview”, Methods Enzymol. 1989; 177:125-31; Wuthrich K. “Protein structure determination in solution by NMR spectroscopy”, J. Biol Chem. 1990 Dec. 25; 265(36):22059-62 and references therein). Upon binding of the small molecule modulator to the protein, changes in the ¹H, ¹³C and ¹⁵N chemical shifts would be observed for those amino acid residues whose local anisotropies are affected by (1) direct contact and/or interaction with the ligand, and/or (2) a conformational movement in the apo-protein upon binding with the ligand. This last point is exemplified by the movement of the Phe of the DFG motif in a kinase from the DFG-“in” conformation to the DFG-“out” conformation. Uniform ¹⁵N and ¹³C isotopic labeling of the protein utilizes modern molecular biology techniques that are standard for one skilled in the art. See Zhao, Q; Frederick, R; Seder, K; Thao, S; Sreenath, H; Peterson, F; Volkman, B. F.; Markley, J. L.; Fox, B. G. “Production in two-liter beverage bottles of proteins for NMR structure determination labeled with either ¹⁵N- or ¹³C-¹⁵N.” J Struct Funct Genomics. 2004; 5(1-2):87-93 and references therein.

For proteins such as kinases whose molecular weights are >20 kD, direct assignment of all ¹H, ¹³C and ¹⁵N resonances within the protein is difficult to achieve. In such cases, specific labeling of key residues identified to be either (1) directly involved in binding of the ligand and/or (2) undergoing a conformational change relative to the apo-protein upon binding to the ligand. Specific labeling of one or more residues within the protein occurs by one of the following methods:

a. Labeling of all examples of a specific amino acid within a protein. This is accomplished through the use of bacterial auxotrophs or cell free in-vitro translation using the labeled amino acid(s) as input.
b. Site specific labeling of a single amino acid using nonsense codon technology either by in-vivo expression or in-vitro translation. See Wang, L; Brock, A; Herberich, B; Schultz, P. G. “Expanding the genetic code of Escherichia coli.”, Science. 2001 Apr. 20; 292(5516):498-500 and references therein.
c. Semi-synthesis or surgical labeling of one or more residues by the combination of in vivo expression and intein technology for the synthesis of peptides and protein. See Hondal, R. J. and Raines, R. T. “Semisynthesis of proteins containing selenocysteine” Methods Enzymol. (2002) 347:70-83; Hondal, R. J.; Nilsson, B. L.; 1 Raines, R. T. “Selenocysteine in native chemical ligation and expressed protein ligation”, J. Am. Chem. Soc. (2001) 123(21):5140-1; Nilsson, B. L.; Soellner, M. B.; Raines, R. T. “Chemical Synthesis of Proteins.”, Annu Rev Biophys Biomol Struct. 2004. Kiick, K. L.; Saxon, E; Tirrell, D. A.; Bertozzi, C. R. “Incorporation of azides into recombinant proteins for chemoselective modification by the Staudinger ligation.”, Proc Natl Acad Sci U S A. 2002 Jan. 8; 99(1):19-24.

This approach is exemplified using Braf kinase. Synthesis of oncogenic V599E Braf kinase having Seq ID NO 45 (MW=31 kD) is accomplished using the combination of in vivo expression of the N- and C-termini of the kinase, which are coupled with specific ¹³C/¹⁵N residues using semi-synthesis methodology to complete the synthesis of the full length kinase. Competency of the enzyme is verified by its use in a biochemical assay.

The semi-synthesis of two different specifically isotopically labeled kinases having SEQ ID NO. 46 and SEQ ID NO. 47 by the semi-synthesis approach yields biochemically competent enzymes that are constitutively active and behave identically to the apo sequence previously expressed in the SF-21 cells.

The first labeled Braf kinase (¹⁵N/¹³C labeled glutamic acid 500, Seq ID NO 46) is chosen because this key residue makes a strong binding contact with the urea moiety of Example 64. Changes in the ¹H, ¹⁵N and ¹³C chemicals shifts of E500 in oncogenic Braf kinase (SEQ ID NO. 46) are observed upon the binding of Example 64. ¹⁵N-filtering is used to show a change in the ¹H chemical shifts upon binding to a small molecule ligand. This demonstrates that changes in the chemical shift of key binding residues can be observed in the presence of Example 64.

The second Braf kinase (¹⁵N/¹³C labeled phenylalanine 594, SEQ ID NO. 47) is chosen to demonstrate how changes in the conformation of protein upon binding of an inhibitor can be observed. Changes in the ¹H, ¹⁵N and ¹³C chemicals shifts of F594 in Braf kinase (SEQ ID NO. 47) are observed upon the binding of Example 64. ¹⁵N-filtering is used to show a change in the ¹H chemical shifts upon binding. This demonstrates that changes in the chemical shift of key binding residues can be observed in the presence of Example 64.

Circular dichroism (CD) is a technique suited for the study of protein conformation (Johnson, W. C., Jr.; Circular Dichroism Spectroscopy and the vacuum ultraviolet region; Ann. Rev. Phys. Chem. (1978) 29:93; Johnson, W. C., Jr.; Protein secondary structure and circular dichroism: A practical guide” Proteins: Str. Func. Gen. (1990) 7:205; Woody, R. W. “Circular dichroism of peptides” (Chapter 2) The Peptides Volume 7 1985 Academic Press; Berova, N., Nakanishi, K., Woody, R. W., Circular Dichroism: Principles and Applications, 2nd Ed. Wiley-VCH, New York, 2000; Schmid, F. X.; Spectral methods of characterizing protein conformation and conformational changes in Protein Structure, a practical approach, edited by T. E. Creighton, IRL Press, Oxford 1989) and in particular has been reported for the study of protein kinase conformation changes. (Bosca, L.; Moran, F.; Circular dichroism analysis of ligand-induced conformational changes in protein kinase C. Mechanism of translocation of the enzyme from the cytosol to the membranes and its implications. Biochemical J (1993) 290:827; Okishio, N.; Tanaka, T.; Fukuda, R.; Nagai, M.; Differential Ligand Recognition by the Src and Phosphatidylinositol 3-Kinase Src Homology 3 Domains: Circular Dichroism and Ultraviolet Resonance Raman Studies; Biochemistry (2003) 42: 208; Deng, Z.; Roberts, D.; Wang, X.; Kemp, R. G.; Expression, characterization, and crystallization of the pyrophosphate-dependent phosphofructo-1-kinase of Borrelia burgdorferi. Arch. Biochem. Biophys. (1999) 371: 326; Reed, J; Kinzel, V; Kemp, B. E.; Cheng, H. C.; Walsh, D. A.; Circular dichroic evidence for an ordered sequence of ligand/binding site interactions in the catalytic reaction of the cAMP-dependent protein kinase. Biochemistry (1985) 24: 2967; Okishio, N.; Tanaka, T.; Nagai, M.; Fukuda, R.; Nagatomo, S.; Kitagawa, T.; Identification of Tyrosine Residues Involved in Ligand Recognition by the Phosphatidylinositol 3-Kinase Src Homology 3 Domain: Circular Dichroism and UV Resonance Ranian Studies., Biochemistry (2001) 40: 15797; Okishio, N.; Tanaka, T.; Fukuda, R; Nagai, M.; Role of the Conserved Acidic Residue Asp21 in the Structure of Phosphatidylinositol 3-Kinase Src Homology 3 Domain: Circular Dichroism and Nuclear Magnetic Resonance Studies, Biochemistry (2001) 40: 119; Mattsson, P. T.; Lappalainen, I.; Backesjo, C.-M.; Brockmann, E.; Lauren, S.; Vihinen, M.; Smith, C. I. E.; “Six X-linked agammaglobulinemia-causing missense mutations in the Src homology 2 domain of Bruton's tyrosine kinase: phosphotyrosine-binding and circular dichroism analysis.” J. Immun. (2000) 164: 4170; Raimbault, C.; Couthon, F.; Vial, C.; Buchet, R.; “Effects of pH and KCl on the conformations of creatine kinase from rabbit muscle. Infrared, circular dichroic, and fluorescence studies.” Euro. J. Biochem. (1995) 234: 570; Shah, J.; Shipley, G. G.; Circular dichroic studies of protein kinase C and its interactions with calcium and lipid vesicles. Biochim. Biophys. Acta (1992) 1119: 19.

The more pronounced helical organization and conformational movements that occur upon kinase activation (upregulation) compared to downregulation states can be observed by CD. Switch control pocket-based small molecule modulation can result in stabilization of a predominant conformational state. Correlation of CD spectra obtained in the presence of small molecular modulators with those obtained in the absence of modulators allows the determination of the nature of small-molecule binding and differentiation of such binding from that of conventional ATP-competitive inhibitors.

A variety of bio-analytical methods can provide small molecule binding affinities to proteins. Affinity-based screening methods using capillary zone electrophoresis (CZE) may be employed in the early stages of screening of candidate small molecule modulators. Direct determination of Kds (dissociation constants) of the small molecule modulator-protein interactions can be obtained. See Heegaard, N. H. H.; Nilsson, S.; Guzman, N. A.; Affinity capillary electrophoresis: important application areas and some recent developments; J. Chromatography B (1998)715: 29-54; Yen-Ho Chu, Y.-H.; Lees, W. J.; Stassinopoulos, A.; Walsh, C. T.; Using Affinity Capillary Electrophoresis To Determine Binding Stoichiometries of Protein-Ligand Interactions, Biochemistry (1994) 3 3:10616-10621; Davis, R. G.; Anderegg R. J.; Blanchard, S. G., Iterative size-exclusion chromatography coupled with liquid chromatographic mass spectrometry to enrich and identify tight-binding ligands from complex mixtures, Tetrahedron (1999) 55: 11653-1166; Shen Hu, S.; Dovichi, N. J.; Capillary Electrophoresis for the Analysis of Biopolymers; Anal. Chem. (2002) 74: 2833-2850; Honda, S.; Taga, A.; Suzuki, K; Suzuki, S.; Kakhi, K, Determination of the association constant of monovalent mode protein-sugar interaction by capillary zone eletrophoresis, J. Chromatography B (1992) 597: 377-382; Colton, I. J.; Carbeck, J. D.; Rao, J.; Whitesides, G. M., Affinity Capillary Electrophoresis: A physical-organic tool for studying interaction in biomolecular recognition, Electrophoresis (1998) 19: 367-382.

Another affinity based screening method makes use of reporter fluoroprobe binding to a candidate protein. Candidate small molecule modulators are screened in this fluoroprobe assay. Compounds which do bind to the protein are measured by a modulation in the fluorescence of the fluoroprobe reporter. This method is reported in the following Example C.

The invention also pertains to small molecule modulator-protein adducts. The proteins are of the type defined previously. Insofar as the modulators are concerned, they should have functional groups complemental with active residues within the switch control pocket regions, in order to maximize modulator-protein binding. For example, in the case of the kinases, it has been found that modulators having 1-3 dicarbonyl linkages are often useful. Where switch control pockets of cationic character are found, the small molecule modulators would often have acidic functional groups or moieties, e.g., sulfonic, phosphonic, or carboxylic groups. In terms of molecular weight, preferred modulators would typically have a molecular weight of from about 120-650 Da, and more preferably from about 300-550 Da. If these small molecule modulators are to be studied in whole cell environments, their properties should conform to well understood principles that optimize the small molecule modulators for cell penetrability (Lipinski's Rule of 5, Advanced Drug Delivery Reviews, Vol. 23, Issues 1-3, pp 3-25 (1997)).

The invention also provides methods of altering the biological activity of proteins broadly comprising the steps of first providing a naturally occurring protein having a switch control pocket. Such a protein is then contacted with a non-naturally occurring molecule modulator under conditions to cause the modulator to bind with the protein at the region of the pocket in order to at least partially regulate the biological activity of the protein by inducing or restricting the conformation of the protein.

The following examples set forth representative methods in accordance with the invention. It is to be understood, however, that these examples are provided by way of illustration and nothing therein should be taken as a limitation upon the overall scope of the invention.

### Example A

In the following steps 1-3, techniques are illustrated for the identification and/or development of small molecules which will interact at the region of switch control pockets forming a part of naturally occurring proteins, in order to modulate the in vivo biological activity of the proteins. Specifically, a family of 8 known kinase proteins are analyzed using the process of the invention, namely the Abl, p38-alpha, wild-type Braf, oncogenic V599E Braf, Gsk-3 beta, insulin receptor-1, protein kinase B/Akt and transforming growth factor B-I receptor kinases. These examples are illustrative of the techniques and are not intended to limit the application thereof.

### Step 1: Identification and Classification of Switch Control Ligands Within the 8 Kinase Proteins

In general, the switch control ligands of the kinases can be identified from using sequence and structural data from the respective kinases, if sufficiently detailed information of this type is available. Thus, this step of the method can be accomplished without experimentation. The known data relative to the kinases permits ready identification of modifiable amino acid residues, which in the case of these proteins are modified by phosphorylation, acylation, methionine or cysteine oxidation, cysteine S-nitrosylation, or cystinylation. The probable extent of the entire switch control ligand sequence can then be deduced. An additional helpful factor in the case of the kinases is that many ligands often begin with a DFG sequence of residues and ends with an APE sequence of residue (the single letter amino acid code is used throughout).

c-Abl Kinase or Bcr-Abl Kinase

The full length human c-Abl isoform 1-B sequence is provided herein as SEQ ID NO. 29. The full length Bcr-Abl sequence is provided herein as SEQ ID NO. 33. One switch control ligand sequence of Abl kinase and bcr-abl fusion protein kinase are constituted by the sequence: D400, F401, G402, L403, S404, R405, L406, M407, T408, G409, D410, T411, Y412, T413, A414, H415 (ligand 1, c-Abl isoform 1-B sequence numbering) (SEQ ID NO. 1). Y412 becomes phosphorylated upon (bcr)Abl activation by upstream regulatory kinases or by autophosphorylation, and thus is a transiently modified residue (Tanis et al, Molecular and Cellular Biology (2003) 23: 3884; Brasher and Van Etten, The Journal of Biological Chemistry (2000) 275: 35631). The switch control ligand sequence begins with DFG and terminates with E428.

An alternate switch control ligand has the sequence Myr-G2, Q3, Q4, P5, G6, K7, V8, L9, G10, D11, Q12, R13, R14, P15, S16, L17 (ligand 2, human c-Abl isoform 1-B sequence numbering) (SEQ ID NO. 2). Ligand 2, specific to the abl kinase isoform 1B, is the N-terminal cap of the Abl protein sequence, and in particular the N-terminal myristolyl group located on G2 (Glycine 2) is the modified amino acid residue (Jackson and Baltimore, (1989) EMBO Journal 8:449; Resh, Biochem Biophys. Acta (1999) 1451:1).

p38-alpha Kinase

The switch control ligand sequence of p38-alpha kinase (SEQ ID NO. 3) is constituted by the sequence: D168, F169, G170, L171, A172, R173, H174, T175, D176, D177, E178, M179, T180, G181, Y182, V183, A184, T185, R186, W187, Y188, R189 (SEQ ID NO. 4). T180 and Y182 become phosphorylated upon p38-alpha activation by upstream regulatory kinases (see Wilson et al, Chemistry & Biology (1997) 4:423 and references therein), and thus are transiently modifiable residues.

### Braf Kinase

The switch control ligand sequence of full length Braf kinase (Seq. ID NO. 40) is constituted by the sequence: D593, F594, G595, L596, A597, T598, V599, K600, S601, R602, W603, S604, G605, S606, H607, Q608, F609, E610, Q611, L612, S613, G614, S615, I616, L617, W618, M619, A620, P621, E622 (SEQ ID NO. 41) T598 and S601 become phosphorylated upon Braf activation by upstream regulatory kinases, and are thus transiently modifiable residues.

### Oncogenic V599E Braf Kinase

The switch control ligand sequence of full length oncogenic V599E Braf kinase (Seq. ID NO. 42) is constituted by the sequence: D593, F594, G595, L596, A597, T598, E599, K600, S601, R602, W603, S604, G605, S606, H607, Q608, F609, E610, Q611, L612, S613, G614, S615, I616, L617, W618, M619, A620, P621, E622 (SEQ ID NO. 43) V599 is mutated to an E599 residue, and this mutated E599 functions as the modifiable residue to activate the switch control ligand independent of phosphorylation of T598 and S601. Thus, V599E is a constituitively activated kinase, wherein E599 provides a surrogate acidic functionality mimicking phosphorylation of T598 and/or S601 to activate the switch control mechanism of Braf kinase.

### Gsk-3 Beta Kinase

The full length Gsk-3 beta kinase sequence is provided herein as SEQ ID No. 31. The Gsk-3 beta kinase sequence corresponding to the 1GNG crystal structure is provided herein as SEQ ID NO. 15. The switch control ligand sequence of Gsk-3 beta kinase protein is constituted by the sequence: D200, F201, G202, S203, A204, K205, Q206, L207, V208, K209, G210, E211, P212, N213, V214, S215, Y216, I217, C218, S219, R220 (Gsk ligand 1) (SEQ ID NO. 5); Y216 becomes phosphorylated upon activation by upstream regulatory kinases (Hughes et al, EMBO Journal (1993) 12: 803; Lesort et al, Journal of Neurochemistry (1999) 72:576; ter Haar et al, Nature Structural Biology (2001) 8: 593 and references therein.

An alternative switch control ligand sequence is: G3, R4, P5, R6, T7, T8, S9, F10, A11, E12 (Gsk ligand 2) (SEQ ID NO. 6); S9 becomes phosphorylated by the action of the upstream kinase PKB/Akt (Dajani et al, Cell (2001) 105: 721) Cross et al, Nature (1995) 378:785). S9 is the transiently modifiable residue.

### Insulin Receptor Kinase-1

The full length IRK-1 gene is provided herein as SEQ ID NO. 34. The sequence corresponding to the 1GAG crystal structure is provided herein as SEQ ID NO. 19. The switch control ligand sequence of insulin receptor kinase-1 is constituted by the sequence: D1150, F1151, G1152, M1153, T1154, R1155, D1156, I1157, Y1158, E1159, T1160, D1161, Y1162, Y1163, R1164, K1165, G1166, G1167, K1168, G1169, L1170 (SEQ ID NO. 7). Y1158, Y1162, and Y1163 are the transiently modifiable residues and become phosphorylated upon activation of the insulin receptor by insulin (see Hubbard et al, EMBO Journal (1997) 16: 5572 and references therein).

### Protein Kinase B/Akt

The full length Akt1 sequence is provided herein as SEQ ID NO. 36. The protein kinase B/Akt kinase-only domain is provided herein as SEQ ID NO. 37. It is noted that these sequences differ at the N and C terminii. Additionally, the kinase-only domain begins at residue 143 of the full length sequence. The switch control ligand sequence of protein kinase B/Akt is constituted by P468, H469, F470, P471, Q472, F473, S474, Y475, S476, A477, S478 (SEQ ID NO. 8). S474 is the transiently modifiable residue which is phosphorylated upon activation by upstream kinase regulatory proteins, thereby increasing PKB/Akt activity 1,000 fold above unphosphorylated PKB/Akt (Yang et al, Molecular Cell (2002) 9:1227 and references therein).

### Transforming Growth Factor B-I Receptor Kinase

The full length sequence of the TGF-B-I receptor kinase is provided herein as SEQ ID NO. 39. The switch control ligand of transforming growth factor B-I receptor kinase is T185, T186, S187, G188, S189, G190, S191, G192, L193, P194, L195, L196 (SEQ ID NO. 9). T185, T186, S187, S189, and S191 are the transiently modifiable residues and are partially or fully phosphorylated upon activation by the kinase activity of Transforming Growth Factor B-II receptor (Wrana et al, Nature (1994) 370: 341; Chen and Weinberg, Proc. Natl. Acad. Sci. USA (1995) 92:1565).

### Step 2: Identification and Classification of Switch Control Pockets

As in the case of identification of the switch control ligands, the complemental switch control pockets may be deduced from published kinase data, and particularly by X-ray crystallography structural analysis. An initial step in this analysis is the identification of residues which will bind with the previously identified modifiable residues within the corresponding switch control ligands. In steps 2 and 3, switch pockets, composite switch pockets, and combined switch pockets are identified. Switch pockets are initially identified from amino acid residues which form the pocket into which the switch control ligand binds. Composite switch pockets are then identified for many kinases, wherein amino acid residues from the switch control ligand sequence are also included in the definition of the switch pocket. Finally, combined switch pockets are identified for many kinases, wherein amino acid residues from the ATP pocket, in particular from the hinge region of the ATP pocket, are included in the definition of the switch pocket.

In some conformational states of kinases that do not have functional ATP pockets, amino acid residues from the beta-strand regions and/or the glycine rich loop contribute to the switch control pocket. In other conformational states wherein a functional ATP pocket is present, some of these amino acid residues from the beta-strand regions and/or the glycine rich loop can alternatively contribute to the ATP pocket and hence the definition of a combined switch control pocket. By way of example, in the inactive conformational state of p38-alpha kinase, tyrosine 35 (from the glycine rich loop) contributes to the definition of the switch control pocket and the composite switch control pocket. In this inactive conformational state, the ATP pocket is deformed and the glycine rich loop is displaced. However, in c-Abl or Bcr-Abl kinase, the corresponding tyrosine 272 from the glycine rich loop is, in some cases, in a conformational state which contributes (as part of the ATP pocket) to the definition of the combined switch control pocket for this kinase.

c-Abl Kinase or Bcr-Abl Kinase

X-ray crystal structural analysis of human Abl kinase 1FPU (SEQ ID NO. 10) (Schlindler et al, Science (2000) 289: 1938) and 1IEP (SEQ ID NO. 11) (Nagar et al, Cancer Research (2002) 62: 4236). The switch control pocket sequence is complemental with the previously identified switch control ligand 1 sequence for this kinase and has a cluster of 2 basic amino acids taken from a combination of the C-alpha helix (residues 300-311) and the catalytic loop (residues 378-387). Specifically, lysine 304 from the C-alpha helix and arginine 381 from the catalytic loop constitute Z residues of the switch control pocket, inasmuch as these residues can stabilize the binding of the transiently modified (phosphorylated) residue Y412 from the switch control ligand. Other predicted amino acid residues which contribute to the switch control pocket include residues from the glycine rich loop (tyrosine 272), the beta-3 strand (A288, K290, D295, M297, E298), the beta-4 strand (I312, L317), the beta-5 strand (V318), the beta-6 strand (I332, T334, E335, F336), other amino acids taken from the C-alpha helix (E301, K304, E305, V308, M309) and other amino acids taken from the catalytic loop (F378, I379, H380, R381, D382, N387). Additionally the E-alpha helix residue L373 and the F-alpha helix residue F435 are predicted to form the base of this pocket.

Table 5 illustrates amino acids from the protein sequence which form the switch control pocket for ligand 1 of c-Abl kinase or (bcr)Abl kinase. All references to amino acid residue numbering are relative to the full length human c-Abl kinase isoform 1B (SEQ ID NO. 29).

TABLE 5

| | | | | | |
|---|---|---|---|---|---|
| Glycine Rich Loop | | | | | |
| Y272 | | | | | |
| Beta Strand 3 | | | | | |
| A288 | K290 | D295 | M297 | E298 | |
| Beta Strand 4 | | | | | |
| I312 | L317 | | | | |
| Beta Strand 5 | | | | | |
| V318 | | | | | |
| Beta Strand 6 | | | | | |
| I332 | T334 | E335 | F336 | | |
| Catalytic Loop | | | | | |
| F378 | I379 | H380 | R381 | D382 | N387 |
| C-alpha helix | | | | | |
| E301 | K304 | E305 | V308 | M309 | |
| E-alpha helix | | | | | |
| L373 | | | | | |
| F-alpha helix | | | | | |
| F435 | | | | | |

X-ray crystal structural analysis of Abl kinase revealed a probable switch control pocket sequence based on structure 1OPL (SEQ ID NO. 12), which is complemental with ligand 2. Table 6 illustrates amino acids from the protein sequence which form the switch control pocket complemental with ligand 2 of (bcr)Abl kinase. The amino acid numbering is taken from the amino acid sequence of human c-Abl kinase isoform 1B.

TABLE 6
SH2 Domain and C-Lobe Helical
Switch Control Pocket

| | | | | |
|---|---|---|---|---|
| A-alpha helix | | | | |
| S152 | R153 | N154 | E157 | Y158 |
| E-alpha helix | | | | |
| A356 | L359 | L360 | Y361 | |
| N-Lobe Loop | | | | |
| N393 | | | | |
| F-alpha helix | | | | |
| L448 | A452 | Y454 | | |
| H-alpha helix | | | | |
| C483 | P484 | V487 | E481 | |
| I-alpha helix | | | | |
| E513 | | | | |
| I-I′ Loop | | | | |
| F516 | Q517 | | | |
| I′-alpha helix | | | | |
| I521 | V525 | L529 | | |

p38-alpha Kinase

X-ray crystal structural analysis of p38-alpha kinase based on structure 1KV2 (SEQ ID NO. 14) (Pargellis, et al.; Nat. Struct. Biol 9 pp. 268-272 (2002) revealed the probable switch control pocket. The switch control pocket for the previously identified switch control ligand sequence has a cluster of 2 basic amino acids taken from a combination of the C-alpha helix (residues 61-78) and the catalytic loop (residues 146-155). Specifically, arginine 67 and/or arginine 70 come from the C-alpha helix, and arginine 149 comes from the catalytic loop and these residues constitute the Z groups for this switch control pocket. Other predicted amino acids which contribute to the switch control pocket include residues from the glycine rich loop (residues 34-36, including the X residue tyrosine 35), amino acids taken from the C-alpha helix (residues 61-78), and amino acids taken from the catalytic loop (residues 146-155). Additionally amino acids taken from F-alpha helix (residues 197-200) form the base of this pocket.

Table 7 illustrates amino acids from the protein sequence which form the switch control pocket

TABLE 7

| | | | | |
|---|---|---|---|---|
| Glycine Rich Loop | | | | |
| Y35 | | | | |
| Beta Strand 5 | | | | |
| K53 | | | | |
| Beta Strand 6 | | | | |
| V83 | I84 | | | |
| Beta Strand 7 | | | | |
| L104 | T106 | | | |
| Catalytic Loop | | | | |
| I146 | H148 | R149 | D150 | N155 |
| C-alpha helix | | | | |
| R67 | R70 | E71 | L74 | M78 |
| E-alpha helix | | | | |
| I141 | | | | |
| F-alpha helix | | | | |
| Y200 | | | | |

### Braf Kinase and Oncogenic V599E Braf Kinase

X-ray crystal structural analysis of Braf kinase 1UWH (SEQ ID NO. 44) and oncogenic V599E Braf kinase 1UWJ (SEQ ID NO. 45) structure (P. T. C. Wan et al, Cell (2004) 116: 855) revealed the probable switch control pocket. The switch control pocket for the previously identified switch control ligand sequence has a basic amino acid taken from the catalytic loop. Specifically, arginine 574 comes from the catalytic loop and constitutes a Z group for this switch control pocket. Other predicted amino acids which contribute to the switch control pocket include residues from the glycine rich loop (residues 466-470), beta strands (strands 3, 5 and 6), amino acids taken from the C alpha-helix (residues 493-507), and amino acids taken from the catalytic loop (residues 571-580). Additionally, residues from the E alpha-helix (L566) and the C-lobe (Y632) form the base of this pocket. Table 8 illustrates amino acids from the protein sequence which forms the switch control pocket. The switch control pocket of oncogenic V599E Braf kinase is identical to wildtype Braf kinase.

TABLE 8

| | | | | | | |
|---|---|---|---|---|---|---|
| Glycine Rich Loop | | | | | | |
| S466 | F467 | V470 | | | | |
| Beta Strand 3 | | | | | | |
| K482 | | | | | | |
| Beta Strand 5 | | | | | | |
| I512 | L513 | | | | | |
| Beta Strand 6 | | | | | | |
| I526 | T528 | | | | | |
| Catalytic Loop | | | | | | |
| I571 | H573 | R574 | D575 | N580 | | |
| C-alpha Helix | | | | | | |
| Q493 | A496 | N499 | E500 | V503 | L504 | T507 |
| E-alpha Helix | | | | | | |
| L566 | | | | | | |
| C-Lobe | | | | | | |
| Y632 | | | | | | |

### Gsk-3 Beta Kinase

X-ray crystal structural analysis of gsk-3 beta kinase reveals the switch control pocket based on structures 1GNG (SEQ ID NO. 15), 1H8F (SEQ ID NO. 16), I109 (SEQ ID NO. 18) and 1O9U (SEQ ID NO. 27 structure with axin peptide having SEQ ID. NO. 28) (Frame et al., Molecular Cell, Vol. 7, pp. 1321-1327 (2001); Dajani et al, Cell, Vol. 105, pp. 721-732 (2001); Dajani et al., EMBO Journal, Vol. 22, pp. 494-501 (2003); and ter Haar, et al., Nature Structural Biology, Vol. 8, pp. 593-596 (2001). The switch control pocket corresponding to the above identified switch control ligand sequences 1 and 2 has a cluster of 2 basic amino acids taken from a combination of the C-alpha helix (residues 96-104), and the catalytic loop (residues 177-186). Specifically, arginine 96 comes from the C-alpha helix, and arginine 180 comes from the catalytic loop. These residues constitute the Z groups for this switch control pocket. Other amino acids which contribute to the switch control pocket include residues from the glycine rich loop (residues 66-68), beta-strand 5 (K85), other residues from the N-lobe (H106, I109, V110), amino acids taken from the C-alpha helix (residues 90-104), and amino acids taken from the catalytic loop (residues 177-186). Additionally amino acids from C-lobe (residues 233-235) form the base of this pocket.

Table 9 illustrates amino acids from the protein sequence which form the switch control pocket.

TABLE 9

| | | | | | |
|---|---|---|---|---|---|
| Glycine rich loop | | | | | |
| F67 | | | | | |
| Beta-strand 5 | | | | | |
| K85 | | | | | |
| N-lobe residues | | | | | |
| H106 | I109 | V110 | | | |
| C-alpha helix | | | | | |
| R96 | E97 | I100 | M101 | L104 | |
| Catalytic loop | | | | | |
| I177 | C178 | H179 | R180 | D181 | N186 |
| E-alpha helix | | | | | |
| L169 | I172 | | | | |
| C-lobe residues | | | | | |
| Y234 | | | | | |

### Insulin Receptor Kinase-1

X-ray crystal structural analysis of the insulin receptor kinase-1 reveals the switch control pocket based on structures 1GAG (SEQ ID NO. 19) and 1IRK (SEQ ID NO. 21) (Parang et al., Nat. Structural Biology, 8, p. 37 (2001); Hubbard et al., Nature, 372, p. 476 (1994). The switch control pocket for the switch control ligand sequence has a cluster of 2 basic amino acids taken from a combination of the C-alpha helix (residues 1037-1054), and the catalytic loop (residues 1127-1137). Specifically, arginine 1039 is contributed from the C-alpha helix, and arginine 1131 is contributed from the catalytic loop. These residues constitute the Z groups for this switch control pocket. Other amino acids which contribute to the switch control pocket include residues from the glycine rich loop (residues 1005-1007), amino acids taken from the C-alpha helix (residues 1037-1054), and amino acids taken from the catalytic loop (residues 1127-1137). Additionally amino acids taken from C-lobe (residues 1185-1187) form the base of this pocket.

Table 10 illustrates amino acids from the protein sequence which form the switch control pocket.

TABLE 10

| | | | | | | | |
|---|---|---|---|---|---|---|---|
| Glycine Rich Loop | | | | | | | |
| F1007 | | | | | | | |
| C-alpha helix | | | | | | | |
| R1039 | E1043 | F1044 | N1046 | E1047 | V1050 | M1051 | F1054 |
| Catalytic Loop | | | | | | | |
| F1128 | V1129 | H1130 | R1131 | D1132 | N1137 | | |
| C-Lobe | | | | | | | |
| V1185 | F1186 | T1187 | | | | | |

### Protein Kinase B/Akt

X-ray crystal structural analysis of protein kinase B/Akt reveals the switch control pocket based on structures 1GZK (SEQ ID NO. 22), 1GZO (SEQ ID NO. 23), and 1GZN (SEQ ID NO. 24) (Yang et al, Molecular Cell (2002) 9:1227. The switch control pocket for the corresponding switch control ligand sequence is constituted of amino acid residues taken from the B-alpha helix (residues 185-190), the C— alpha helix (residues 194-204) and the beta-5 strand (residues 225-231). In particular, arginine 202 comes from the C— alpha helix and constitutes a Z group for this switch control pocket.

Table 11 illustrates amino acids from the protein sequence which form the switch control pocket of protein kinase B/Akt.

TABLE 11

| | | | | | | | |
|---|---|---|---|---|---|---|---|
| B-alpha Helix | | | | | | | |
| K185 | E186 | Y187 | I188 | I189 | A190 | | |
| C-alpha Helix | | | | | | | |
| V194 | A195 | H196 | T197 | V198 | T199 | E200 | S201 |
| R202 | V203 | L204 | | | | | |
| beta-5 strand | | | | | | | |
| L225 | C226 | F227 | V228 | M229 | E230 | Y231 | |

### Transforming Growth Factor B-I Receptor Kinase

X-ray crystal structural analysis of the transforming growth factor B-I receptor kinase reveals the switch control pocket, based on structure 1B6C (SEQ ID NO. 25) (Huse et al., Cell (1999) 96:425). The switch control pocket is made up of amino acid residues taken from the GS-1 helix, the GS-2 helix, N-lobe residues 253-266, and C-alpha helix residues 242-252.

Table 12 illustrates amino acids from the protein sequence which form the switch control pocket of TGF B-1 receptor kinase.

TABLE 12

| | | | | | | | |
|---|---|---|---|---|---|---|---|
| GS-1 Helix | | | | | | | |
| Y182 | I181 | | | | | | |
| GS-2 Helix | | | | | | | |
| Q198 | | | | | | | |
| N-LOBE | | | | | | | |
| M253 | L254 | R255 | F262 | I263 | A264 | A265 | D266 |
| C-alpha Helix | | | | | | | |
| W242 | F243 | A246 | Y249 | Q250 | V252 | | |

A second switch control pocket exists in the TGF B-1 receptor kinase. This switch control pocket is similar to the pockets described above for Abl kinase (Table 5), p38-alpha kinase (Table 7), and gsk-3 beta kinase (Table 9). Although TGF B-1 does not have an obvious complementary switch control ligand to match this pocket, nevertheless this pocket has been evolutionarily conserved and may be used for binding small molecule switch control modulators. This pocket is made up of residues from the Glycine Rich Loop, the C-alpha helix, the catalytic loop, the switch control ligand sequence and the C-lobe.

Table 13 illustrates amino acids from the protein sequence which form this switch control pocket.

TABLE 13

| | | | | | |
|---|---|---|---|---|---|
| Glycine rich | | | | | |
| Loop | | | | | |
| R215 | F216 | | | | |
| N-Lobe | | | | | |
| F234 | R237 | | | | |
| C-alpha Helix | | | | | |
| R244 | S241 | I248 | V252 | | |
| Catalytic Loop | | | | | |
| I329 | A330 | H331 | R332 | D333 | L334 |
| C-Lobe | | | | | |
| H392 | F393 | E394 | | | |

A third switch control pocket is spatially located between the ATP binding pocket and the C-alpha helix and is constituted by residues taken from those identified in Table 14. This pocket is provided as a result of the distortion of the C-alpha helix in the “closed form” that binds the inhibitory protein FKBP12 (SEQ ID NO. 26) (see Huse et al, Molecular Cell (2001) 8:671).

Table 14 illustrates the sequence of the third switch control pocket.

TABLE 14

| | | | | | | | |
|---|---|---|---|---|---|---|---|
| Glycine rich Loop | | | | | | | |
| F216 | G217 | V219 | | | | | |
| N-lobe | | | | | | | |
| K232 | F234 | S235 | S236 | L254 | I259 | L260 | G261 |
| F262 | L276 | L278 | S280 | | | | |
| C-alpha Helix | | | | | | | |
| E245 | A246 | I248 | Y249 | V252 | | | |

### Step 3. Ascertain the Nature of the Switch Control Ligand-Switch Control Pocket Interaction, and Identify Appropriate Loci for Small Molecule Design

1. General computational methods. Computer-assisted delineation of switch-control pockets and switch control pocket/ligand interactions utilized modified forms of SurfNet (Laskowsi, R. A, J. Mol. Graph., 1995, 13, 323; PASS; G. Patrick Brady, G. P. Jr.; Stouten, P. F. W., J. Computer-Aided Mol. Des. 2000, 14, 383, Voidoo, G. J. Kleywegt & T. A. Jones (1994) Acta Ctyst D50, 178-185; http://www.iucr.ac.uk/ioumals/acta/tocs/actad/1994/actad5002.html; and Squares; Jiang, F.; Kim, S.-H.; “‘Soft-docking’”: Matching of Molecular Surface Cubes”, J. Mol. Biol. 1991, 219, 79) in tandem with GRASP for pocket visualization (http://trantor.bioc.columbia.edu/grasp/). Panning and docking of small molecule chemotypes into these putative sites employs SoftDock (http://www.scripps.edu/pub/olson-web/doc/autodock/; Morris, G. M.; Goodsell, D. S.; Halliday, R. S.; Huey, R.; Hart, W. E.; Belew, R. K; Olson, A. J, J. Computational Chemistry, 1998, 19, 1639] and Dock [http://www.cmpharm.ucsf.edu/kuntz/dock.html; Ewing, T. D. A.; Kuntz, I. D., J. Comp. Chem. 1997, 18, 1175] with AMBER-based[http://www.amber.ucsf.edu/amber/amber.html] constrained molecular dynamics as appropriate.

The general approach used by pocket analysis programs is to define gap regions and use these to determine what solvent accessible holes are available on the surface of the protein. Gap regions are either based on spheres or squares and are defined by first filling the region between two or more atoms with spheres or squares (whole and truncated) and then using these to compute a 3D density map which, when contoured, defines the surface of the gap region. The general approach, as taken from the Surfnet users manual is defined for spheres as follows:

a. Two atoms, A and B, have a trial gap sphere placed midway between their van der Waals surfaces and just touching each one.

b. Neighboring atoms are then considered in turn. If any penetrate the gap sphere, the trial gap sphere radius is reduced until it just touches the intruding atom. The process is repeated until all the neighboring atoms have been considered. If the radius of the sphere falls below some predetermined minimum limit (usually 1.0 A) it is rejected. Otherwise, the final gap sphere is saved.

c. The procedure is continued until all pairs of atoms have been considered and the gap region is filled with spheres.

d. The spheres are then used to update points on a 3D array of grid-points using a Gaussian function.

e. The update is such that, when the grid is contoured at a contour level of 100.0, the resultant 3D surface corresponds to each gap sphere.

f. When all the spheres have updated the grid, the final 3 D contour represents the surface of the interpenetrating gap spheres, and hence defines the extent of the pocket group of atoms comprising the surface pocket.

Those factors that affect the pocket analysis include the spacing of the grid points, the contour level employed, and the minimum and maximum limits of the sphere radii used to pack the gap. In general, the size and shape of a switch control pocket is described as the consensus pocket found by overlaying the computed switch control pockets determined from each individual program.

As noted above, it has been found that the interaction of a switch control ligand and one or more switch control pockets forms what is termed a “composite switch pocket.” This composite switch pocket has a sequence including amino acid residues taken from both the switch control ligand and the switch control pocket(s).

In other cases, the switch control pocket or the composite switch control pocket may overlap with an active site pocket (e.g., the ATP pocket of a kinase) creating a “combined switch control pocket.” These combined switch control pockets can also be useful as loci for binding with small molecules serving as switch control inhibitors.

Of course, the analysis of composite switch pockets and combined switch pockets is carried out using the same techniques as described above in connection with the switch control pockets.

c-Abl Kinase and Bcr-Abl Kinase

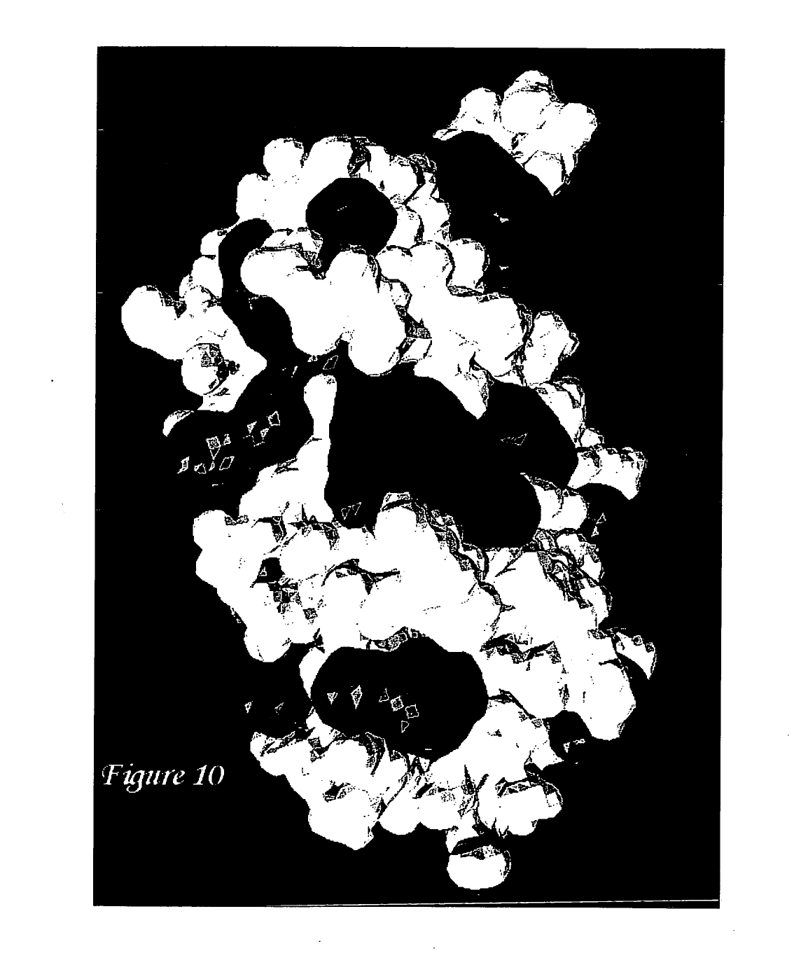

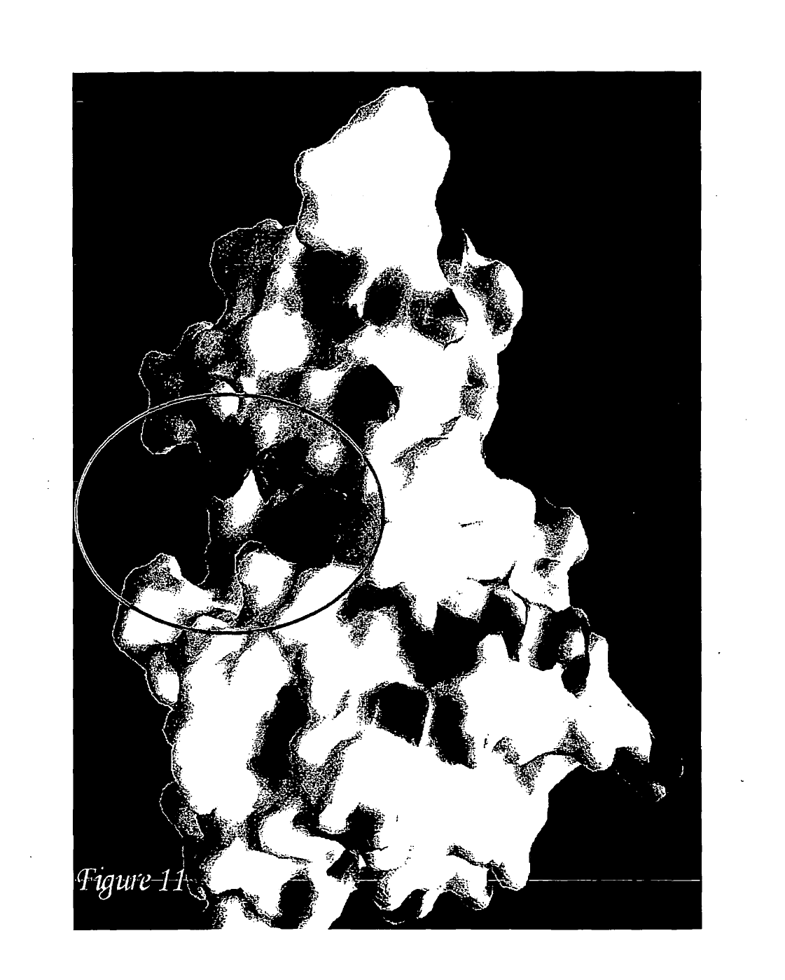

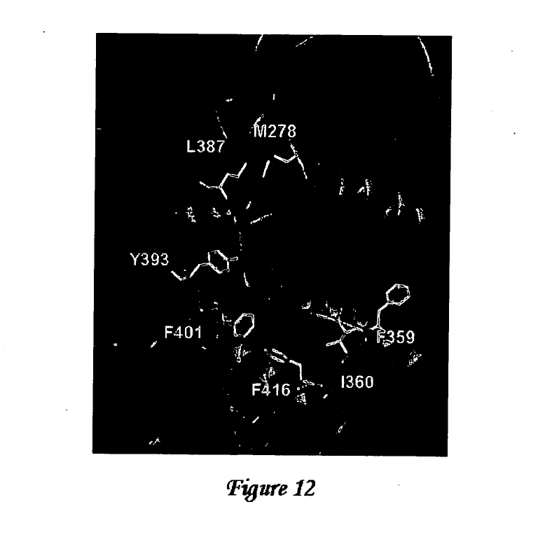

A SURFNET view of the pocket analysis is illustrated in FIG. 10. The switch control pocket is highlighted in light blue. A GRASP view of this switch control pocket is illustrated in FIG. 11, and wherein the composite pocket region of the protein is encircled. FIG. 12 illustrates key amino acid residues which make up the composite switch control pocket of c-Abl kinase or (bcr)Abl kinase. The amino acid residues making up the composite pocket are contributed by the switch control ligand and the switch control pocket previously identified A schematic representation of a composite switch control pocket is depicted in FIG. 6.

The specific amino acid residues making up the composite pocket are set forth in Table 15.

TABLE 15

| | | | | | | |
|---|---|---|---|---|---|---|
| Glycine Rich Loop | | | | | | |
| Y272 | | | | | | |
| Beta-Strand 3 | | | | | | |
| A288 | K290 | D295 | M297 | E298 | | |
| Beta-Strand 4 | | | | | | |
| I312 | L317 | | | | | |
| Beta-Strand 5 | | | | | | |
| V318 | | | | | | |
| Beta-Strand 6 | | | | | | |
| I332 | T334 | E335 | F336 | | | |
| Catalytic Loop | | | | | | |
| F378 | I379 | H380 | R381 | D382 | N387 | |
| C-alpha helix | | | | | | |
| E301 | K304 | E305 | V308 | M309 | | |
| Switch Control Ligand | | | | | | |
| D400 | F401 | G402 | L403 | S404 | R405 | L406 |
| M407 | T408 | G409 | D410 | T411 | Y412 | T413 |
| A414 | H415 | A416 | G417 | A418 | K419 | F420 |
| P421 | I422 | K423 | W424 | T425 | | |
| E-alpha helix | | | | | | |
| L373 | | | | | | |
| F-alpha helix | | | | | | |
| F435 | | | | | | |

Arginine 405 from the switch control ligand sequence contributes a Z group to the composite switch control pocket. The initial small molecule design for this composite switch control pocket focused on chemical probes which would bind to amino acids taken from beta-strands 3, 4, 5, and 6 (see table 15), C-alpha helix (E301, K304, E305, V408, M309), the E-alpha helix (L373), the Catalytic Loop (F378, 1379, H380, R381, D382, N387), and the switch control ligand sequence (D400, F401, G402, R405). Utilization of this composite switch control pocket allowed the design of inhibitors that anchor into this composite switch control pocket of c-Abl kinase or Bcr-Abl kinase.

Representative compounds selected for screening include 1-(3-tert-butyl-1,2,3,4-tetrahydroisoquinolin-7-yl)-1H-pyrazol-5-yl)-3-(2,3-dichlorophenyl)urea (Example 64); (3S)-6-(3-tert-butyl-5-(3-(2,3-dichlorophenyl)ureido)-1H-pyrazol-1-yl)-1,2,3,4-tetrahydroisoquinoline-3-carboxylic acid (Example 65); 1-(3-tert-butyl-1-(1,2,3,4-tetrahydroisoquinolin-6-yl)-1H-pyrazol-5-yl)-3-(2,3-dichlorophenyl)urea (Example 66); and N-(4-methyl-3-(4-phenylpyrimidin-2-ylamino)phenyl)-L4-(2-oxo-4-phenyl-oxazolidinyl-3-carbonyl)benzamide (Example 97).

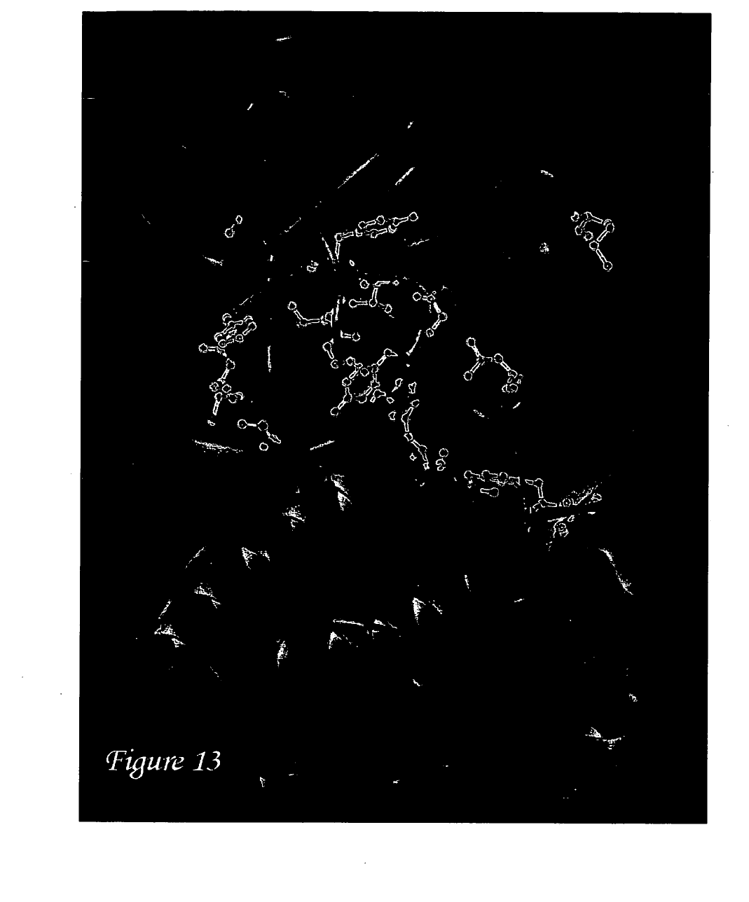

FIG. 13 illustrates key amino acid residues which make up the combined switch control pocket of c-Abl kinase or (bcr)Abl kinase. The amino acid residues making up the combined pocket are contributed by the switch control ligand, the switch control pocket, and the ATP active site previously identified. A schematic representation of a combined switch control pocket is depicted in FIG. 7.

The specific amino acid residues making up the combined pocket are set forth in Table 16. The asterisked sequences indicate regions where amino acid residues contribute as part of the composite switch control pocket in some conformational states of c-Abl or Bcr-Abl kinase, whereas in other conformational states of these kinases, these regions may contribute as part of the ATP pocket to the combined switch control pocket.

TABLE 16

| | | | | | | |
|---|---|---|---|---|---|---|
| Glycine Rich Loop* | | | | | | |
| Y272 | | | | | | |
| Beta Strand 3* | | | | | | |
| A288 | K290 | D295 | M297 | E298 | | |
| Beta Strand 4 | | | | | | |
| I312 | L317 | | | | | |
| Beta Strand 5* | | | | | | |
| V318 | | | | | | |
| Beta Strand 6* | | | | | | |
| I332 | T334 | E335 | F336 | | | |
| Catalytic Loop | | | | | | |
| F378 | I379 | H380 | R381 | D382 | N387 | |
| C-alpha helix | | | | | | |
| E301 | K304 | E305 | V308 | M309 | | |
| Switch Control Ligand | | | | | | |
| D400 | F401 | G402 | L403 | S404 | R405 | L406 |
| M407 | T408 | G409 | D410 | T411 | Y412 | T413 |
| A414 | H415 | A416 | G417 | A418 | K419 | F420 |
| P421 | I422 | K423 | W424 | T425 | | |
| E-alpha helix | | | | | | |
| L373 | | | | | | |
| F-alpha helix | | | | | | |
| F435 | | | | | | |
| ATP Pocket * | | | | | | |
| L267 | G268 | G269 | G270 | Q271 | Y272 | G273 |
| V275 | E277 | M337 | T338 | G340 | N341 | |

Utilization of this combined switch control pocket allowed the design of inhibitors that anchor into this combined switch control pocket of (bcr)Abl kinase.

Representative compounds selected for screening include 1-(1-(3-(2-amino-2-oxoethyl)phenyl)-3-tert-butyl-1H-pyrazol-5-yl)-3-(3-(pyridin-3-yloxy)phenyl)urea (Example 93) and 1-(3-tert-butyl-1-(1,2,3,4-tetrahydroisoquinolin-6-yl)-1H-pyrazol-5-yl)-3-(4-methyl-3-(pyrimidin-2-ylamino)phenyl)urea (Example 94).

p38-alpha Kinase

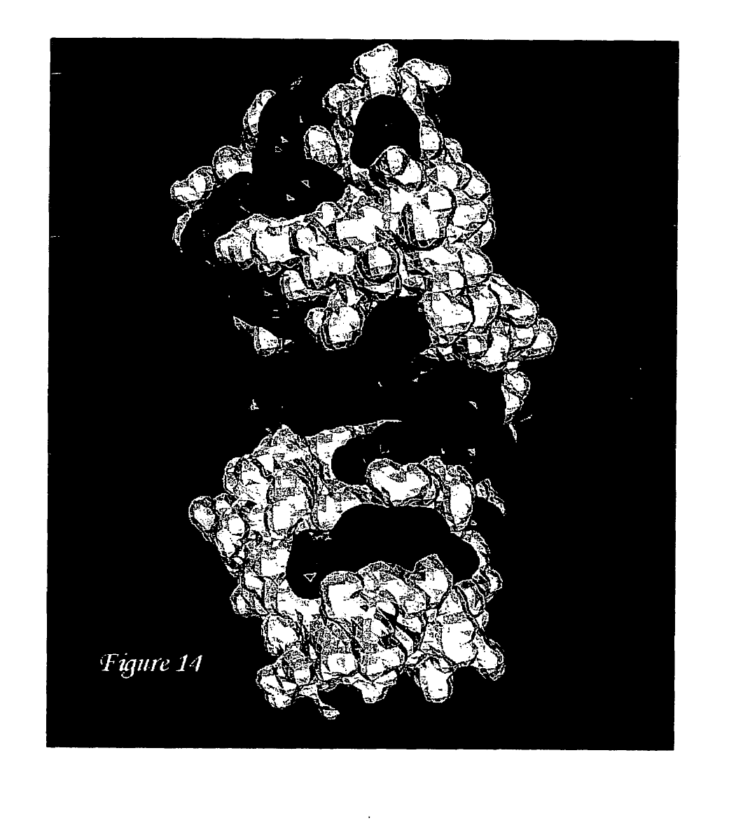

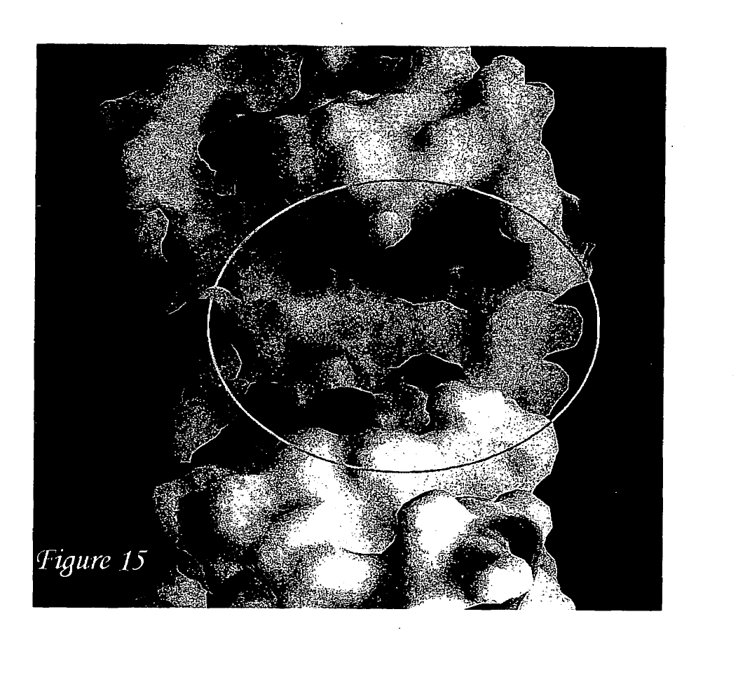

A SURFNET view of the pocket analysis is illustrated in FIG. 14. The composite switch control pocket is highlighted in light blue. A GRASP view of this composite switch control pocket is illustrated in FIG. 15.

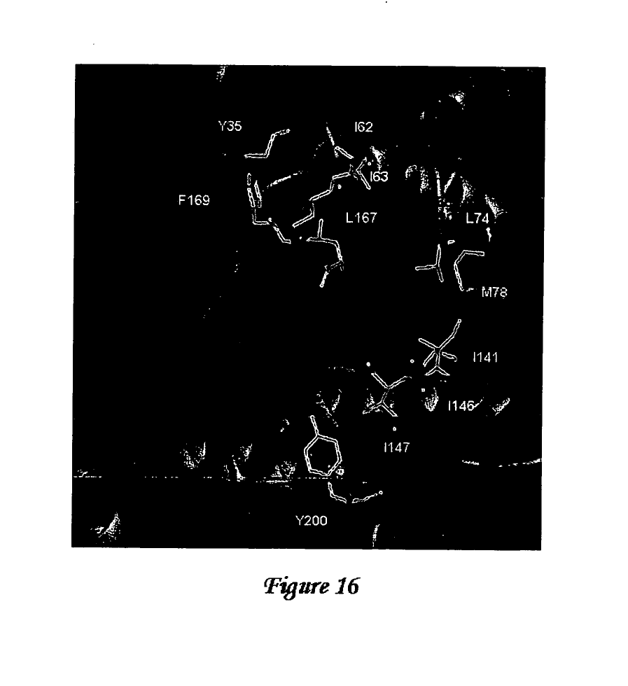

FIG. 16 illustrates key amino acid residues which make up the composite switch control pocket of p38-alpha kinase. These amino acids are taken from the glycine rich loop (Y35), the C-alpha helix (I62, I63, R67, R70, L74, L75, M78), beta-strands 5-7 (K53, V83, I84, L104, T106), the E-alpha helix (I141, I146), the catalytic loop (I147, H148, R149, D150, N155), an N-Lobe strand (L167), the switch control ligand sequence (D168, F169, H174), and the F-alpha helix (Y200). The specific amino acid residues making up the composite pocket are set forth in Table 17.

TABLE 17

| | | | | | | |
|---|---|---|---|---|---|---|
| Glycine Rich Loop | | | | | | |
| Y35 | | | | | | |
| Beta-Strand 5 | | | | | | |
| K53 | | | | | | |
| Beta-Strand 6 | | | | | | |
| V83 | I84 | | | | | |
| Beta-Strand 7 | | | | | | |
| L104 | T106 | | | | | |
| Catalytic Loop | | | | | | |
| I146 | H148 | R149 | D150 | N155 | | |
| C-alpha helix | | | | | | |
| R67 | R70 | E71 | L74 | M78 | | |
| Switch Control Ligand | | | | | | |
| D168 | F169 | G170 | L171 | A172 | R173 | H174 |
| T175 | D176 | D177 | E178 | M179 | T180 | G181 |
| Y182 | V183 | A184 | T185 | R186 | W187 | Y188 |
| R189 | | | | | | |
| E-alpha helix | | | | | | |
| I141 | | | | | | |
| F-alpha helix | | | | | | |
| Y200 | | | | | | |

Utilization of this composite switch control pocket allows the design of inhibitors that anchor into this switch control pocket of p38-alpha kinase.

Representative compounds include:
1-(3-tert-butyl-1-(3-(2-morpholino-2-oxoethyl)phenyl)-1H-pyrazol-5-yl)-3-(naphthalen-1-yl)urea (Example 21);
1-(1-(3-(2-amino-2-oxoethyl)phenyl)-3-tert-butyl-1H-pyrazol-5-yl)-3-(4-chlorophenyl)urea (Example 26);
3-(3-(3-tert-butyl-5-(3-(naphthalen-1-yl)ureido)-1H-pyrazol-1-yl)phenyl)propanoic acid (Example 29);
3-(3-(3-tert-butyl-5-(3-(4-chlorophenyl)ureido)-1H-pyrazol-1-yl)phenyl)propanoic acid (Example 30);
3-(4-(3-tert-butyl-5-(3-(naphthalen-1-yl)ureido)-1H-pyrazol-1-yl)phenyl)propanoic acid (Example 31); and
3-(4-(3-tert-butyl-5-(3-(4-chlorophenyl)ureido)-1H-pyrazol-1-yl)phenyl)propanoic acid (Example 32).

The amino acid residues making up the combined pocket for p38-alpha kinase are contributed by the switch control ligand, the switch control pocket, and the ATP pocket. A schematic representation of a combined switch control pocket is depicted in FIG. 7.

The specific amino acid residues making up the combined pocket are set forth in Table 18. The asterisked sequences indicate regions where amino acid residues contribute as part of the composite switch control pocket in some conformational states of p38-alpha kinase, whereas in other conformational states of p38-alpha kinase, these regions may contribute as part of the ATP pocket to the combined switch control pocket.

TABLE 18

| | | | | | | |
|---|---|---|---|---|---|---|
| Glycine Rich Loop* | | | | | | |
| Y35 | | | | | | |
| Beta-Strand 5* | | | | | | |
| K53 | | | | | | |
| Beta-Strand 6* | | | | | | |
| V83 | I84 | | | | | |
| Beta-Strand 7* | | | | | | |
| L104 | T106 | | | | | |
| Catalytic Loop | | | | | | |
| I146 | H148 | R149 | D150 | N155 | | |
| C-alpha helix* | | | | | | |
| R67 | R70 | E71 | L74 | M78 | | |
| Switch Control Ligand | | | | | | |
| D168 | F169 | G170 | L171 | A172 | R173 | H174 |
| T175 | D176 | D177 | E178 | M179 | T180 | G181 |
| Y182 | V183 | A184 | T185 | R186 | W187 | Y188 |
| R189 | | | | | | |
| E-alpha helix | | | | | | |
| I141 | | | | | | |
| F-alpha helix | | | | | | |
| Y200 | | | | | | |

### Braf Kinase

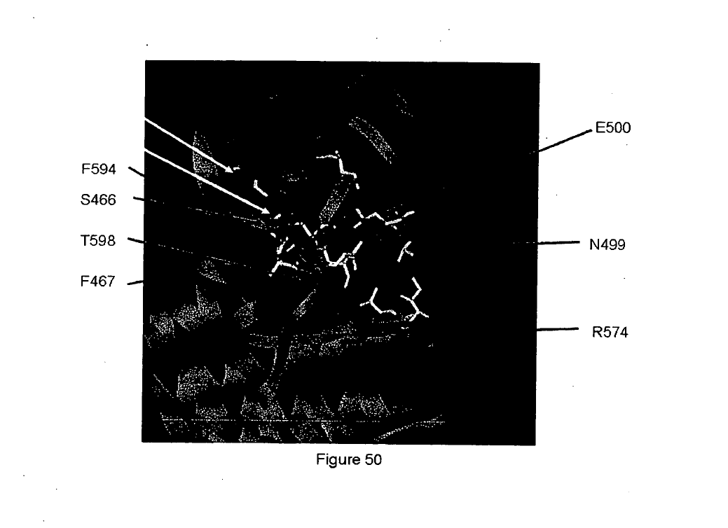

FIG. 50 illustrates key amino acid residues which make up the composite switch control pocket of Braf kinase. The switch control ligand sequence (D593, F594, G595, L596, A597, T598, V599, K600, S601, R602) contributes to the composite switch control pocket for Braf kinase, along with residues taken from the glycine rich loop (residues 466-470), beta strands (strands 3, 5 and 6), the C alpha-helix (residues 493-507), and amino acids taken from the catalytic loop (residues 571-580). Additionally, residues from the E alpha-helix (L566) and the C-lobe (Y632) form the base of this pocket. R574 and R602 are the Z groups (as defined in FIG. 4a) which stabilize the binding of transiently phosphorylated switch ligand residues phospho-T598 or phospho-S601. The specific amino acid residues making up the composite pocket are set forth in Table 19.

TABLE 19

| | | | | | | | |
|---|---|---|---|---|---|---|---|
| Glycine Rich | | | | | | | |
| Loop | | | | | | | |
| S466 | F467 | V470 | | | | | |
| Beta Strand 3 | | | | | | | |
| K482 | | | | | | | |
| Beta Strand 5 | | | | | | | |
| I512 | L513 | | | | | | |
| Beta Strand 6 | | | | | | | |
| I526 | T528 | | | | | | |
| Catalytic Loop | | | | | | | |
| I571 | H573 | R574 | D575 | N580 | | | |
| C-alpha Helix | | | | | | | |
| Q493 | A496 | N499 | E500 | V503 | L504 | T507 | |
| Switch Control | | | | | | | |
| Ligand | | | | | | | |
| D593 | F594 | G595 | L596 | A597 | T598 | V599 | K600 |
| S601 | R602 | W603 | S604 | K605 | S606 | H607 | Q608 |
| F609 | E610 | Q611 | L612 | S613 | G614 | S615 | I616 |
| L617 | W618 | M619 | A620 | P621 | E622 | | |
| E-alpha Helix | | | | | | | |
| L566 | | | | | | | |
| C-Lobe | | | | | | | |
| Y632 | | | | | | | |

### Oncogenic V599E Braf Kinase

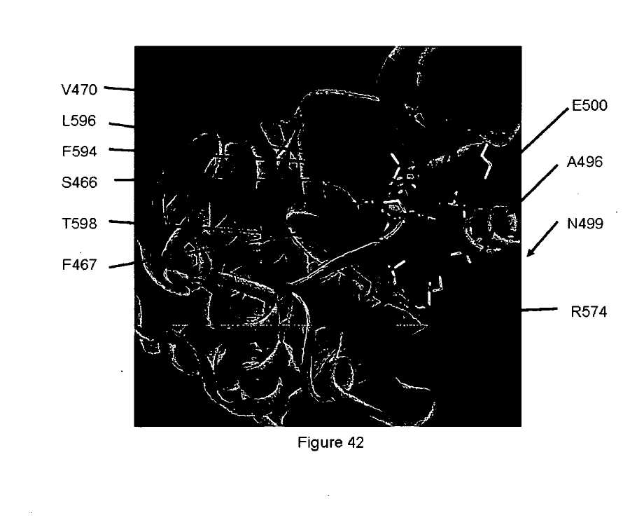

FIG. 42 illustrates key amino acid residues which make up the composite switch control pocket of oncogenic V599E Braf kinase. These amino acids are taken from the glycine rich loop (S466, F467, V470), beta-strand 3 (K482), beta-strand 5 (I512, L513), beta-strand 6 (I526, T528), the C-alpha helix (Q493, A496, N499, E500, V503, L504, T507), the catalytic loop (1571, H573, R574, D575, N580), the switch control ligand sequence (D593, F594, G595, L596, A597, T598, E599, K600, S601, R602), the E alpha-helix (L566), and the C-lobe residue (Y632). R574 and R602 are the Z groups (as defined in FIG. 4a) which stabilize the binding of the mutated switch ligand residue E599. The specific amino acid residues making up the composite pocket are set forth in Table 20.

TABLE 20

| | | | | | | | |
|---|---|---|---|---|---|---|---|
| Glycine Rich | | | | | | | |
| Loop | | | | | | | |
| S466 | F467 | V470 | | | | | |
| Beta Strand 3 | | | | | | | |
| K482 | | | | | | | |
| Beta Strand 5 | | | | | | | |
| I512 | L513 | | | | | | |
| Beta Strand 6 | | | | | | | |
| I526 | T528 | | | | | | |
| Catalytic Loop | | | | | | | |
| I571 | H573 | R574 | D575 | N580 | | | |
| C-alpha Helix | | | | | | | |
| Q493 | A496 | N499 | E500 | V503 | L504 | T507 | |
| Switch Control | | | | | | | |
| Ligand | | | | | | | |
| D593 | F594 | G595 | L596 | A597 | T598 | E599 | K600 |
| S601 | R602 | W603 | S604 | K605 | S606 | H607 | Q608 |
| F609 | E610 | Q611 | L612 | S613 | G614 | S615 | I616 |
| L617 | W618 | M619 | A620 | P621 | E622 | | |
| E-alpha Helix | | | | | | | |
| L566 | | | | | | | |
| C-Lobe | | | | | | | |
| Y632 | | | | | | | |

Representative examples of Braf kinase or oncogenic V599E Braf kinase inhibitors include 1-(3-tert-butyl-1-(1,2,3,4-tetrahydroisoquinolin-7-yl)-1H-pyrazol-5-yl)-3-(2,3-dichlorophenyl)urea (Example 64) and (3S)-6-(3-tert-butyl-5-(3-(2,3-dichlorophenyl)ureido)-1H-pyrazol-1-yl)-1,2,3,4-tetrahydroisoquinoline-3-carboxylic acid (Example 65).

### Gsk-3 Beta Kinase

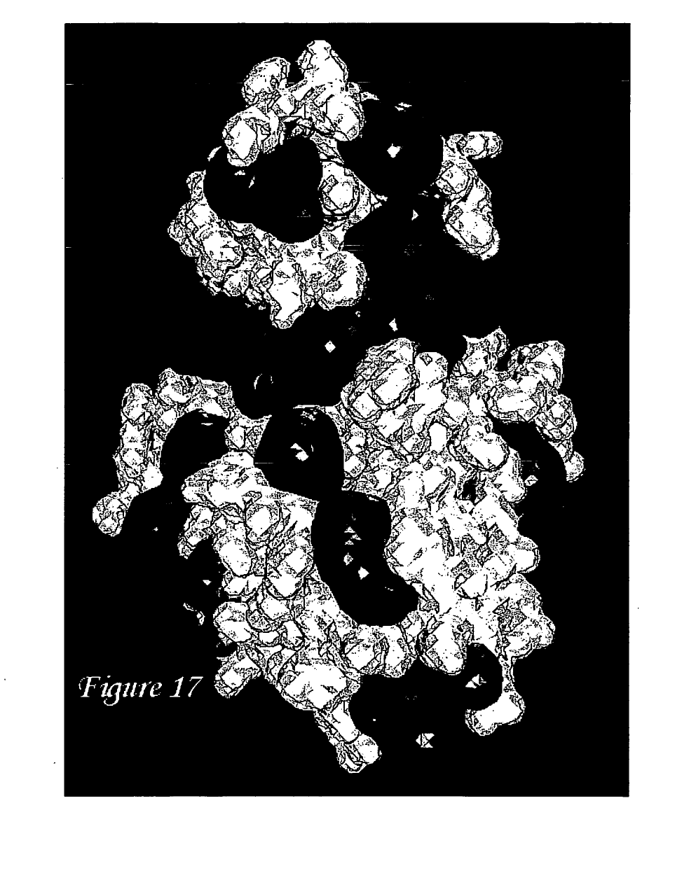

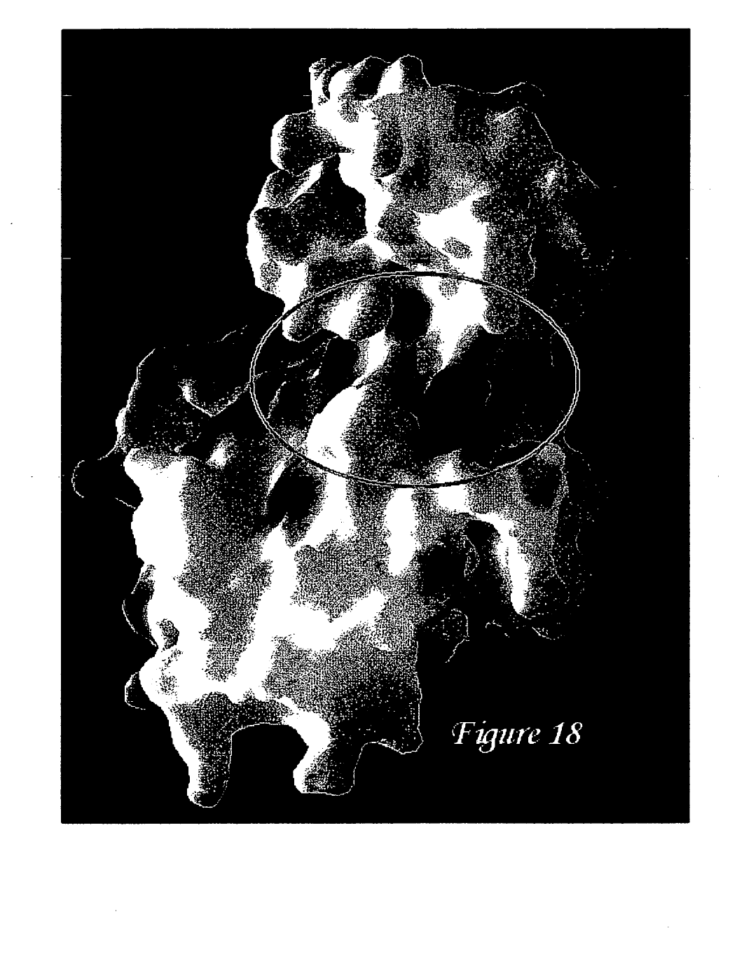

A SURFNET view of the pocket analysis is illustrated in FIG. 17. The composite switch control pocket is highlighted in light blue. A GRASP view of this composite switch control pocket is illustrated in FIG. 18.

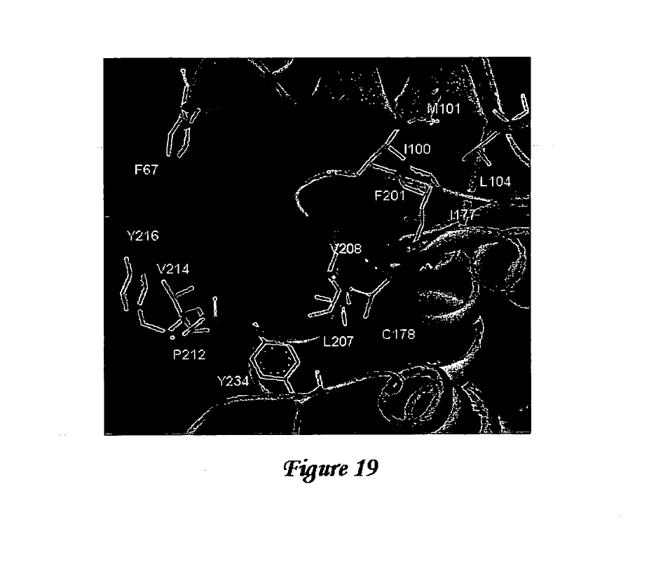

FIG. 19 illustrates key amino acid residues which make up the composite switch control pocket of gsk-3 beta kinase having SEQ ID NO. 16. The residues are from the glycine rich loop (F67), beta-strand 5 (K85), other N-lobe residues (H106, I109, V110), the C-alpha helix (R96, E97, I100, M101, K103, L104), the E-alpha helix (L169, I172), the catalytic loop (I177, C178, H179, R180, D181, N186), the switch control ligand sequence (D200, F201, S203, K205, L207, V208, P212, N213, V214, Y216), and the C-Lobe residue (Y234). Utilization of this pocket allows the design of small molecule modulator compounds that anchor into this composite switch control pocket of gsk-3 beta kinase.

The composite pocket illustrated in Table 21 is a dual-functionality switch control pocket. When it binds with complemental ligand sequence 1 (Gsk ligand 1) the pocket functions as an on-pocket upregulating protein activity. Alternately, when it binds with complemental ligand sequence 2 (Gsk ligand 2) the pocket functions as an off-pocket downregulating protein activity.

Table 21 illustrates amino acids from the protein sequence which form the composite switch control pocket.

TABLE 21

| | | | | | |
|---|---|---|---|---|---|
| Glycine rich Loop | | | | | |
| F67 | | | | | |
| Beta-strand 5 | | | | | |
| K85 | | | | | |
| N-lobe residues | | | | | |
| H106 | I109 | V110 | | | |
| C-alpha helix | | | | | |
| R96 | E97 | I100 | M101 | L104 | |
| Catalytic loop | | | | | |
| I177 | C178 | H179 | R180 | D181 | N186 |
| Switch control ligand | | | | | |
| D200 | F201 | G202 | S203 | A204 | K205 |
| Q206 | L207 | V208 | R209 | G210 | E211 |
| P212 | N213 | V214 | S215 | Y216 | I217 |
| C218 | S219 | R220 | | | |
| E-alpha helix | | | | | |
| L169 | I172 | | | | |
| C-lobe residues | | | | | |
| Y234 | | | | | |

### Example B

### Step 4: Express and Purify the Proteins Statically Confined to Their Different Switch Controlled States

Gene Synthesis. Genes were completely prepared from synthetic oligonucleotides with codon usage optimized using software (Gene Builder™) provided by Emerald/deCODE genetics, Inc. Whole gene synthesis allowed the codon-optimized version of the gene to be rapidly synthesized. Strategic placement of restriction sites facilitated the rapid inclusion of additional mutations as needed.

The proteins were expressed in baculovirus-infected insect cells or in E. coli expression systems. The genes were optionally modified by incorporating affinity tags that can often allow one-step antibody-affinity purification of the tagged protein. The constructs were optimized for crystallizability, ligand interaction, purification and codon usage. Two 11 Liter Wave Bioreactors for insect cell culture capacity of over 100 L per month were utilized.

Protein purification. For protein purification, an AKTA Purifier, AKTA FPLC, Parr Nitrogen Cavitation Bomb, EmulsiFlex-C5 homogenizer and Protein Maker™ Protein Maker (Emerald's automated parallel purification system) were utilized. Instrumentation for characterizing purified protein included fluorescence spectroscopy, MALDI-ToF mass spectrometry, and dynamic light scattering.

Total cell paste was disrupted by nitrogen cavitation, French press, or microfluidization. The extracts were subjected to parallel protein purification using the Protein Maker device. The Protein Maker is a robotic device developed by Emerald that performs simultaneous purification columns in run multiple runs (including Glu-mAb, metal chelate, Q-seph, S-Seph, Phenyl-Seph, and Cibacron Blue) in parallel. The fractions were analyzed by SDS-PAGE. Purified protein was subjected to a number of biophysical assays (Dynamic Light Scattering, UV absorption, MALDI-ToF, analytical gel filtration etc) to quantitate the level of purity.

### Abl Kinase

Whole gene synthesis and subcloning of Abl kinase having SEQ ID NO. 56 (kinase domain, 6×His-TEV tag, Residues 248-534), Abl kinase having SEQ ID NO. 53 (kinase domain, Glu-6×His-TEV tag, Residues 248-518), and Abl kinase having SEQ ID NO. 51 (isoform 1-B 1-531 with Y412F) was completed and transfections were performed in insect cells. Bcr-abl kinase having SEQ ID NO. 59 (Glu-6×His-TEV tag, Residues 1-2029) and bcr-abl kinase having SEQ ID NO. 60 (Glu-6×His-TEV tag, Residues 1-2029; Y412F mutant) were similarly prepared and transfected into insect cells. Fernbach transfection cultures were optionally performed in the presence of the ATP competitive inhibitor PD 180970 or Gleevec to ensure that (bcr) Abl proteins produced were not phosphorylated at Y245 or Y412 (see Tanis et al. Molecular Cell Biology, Vol. 23, p 3884, (2003); Van Etten et al., Journal of Biological Chemistry, Vol. 275, p 35631, (2000)). Protein expression levels were determined by immunoprecipitation and SDS-Page. Protein expression levels for Abl kinases exceeded 10 mg/L. Py20 (anti-phosphotyrosine antibody) Western blotting was performed on purified protein expressed in the presence of these inhibitors to ensure that Y245 or Y412 was not phosphorylated.

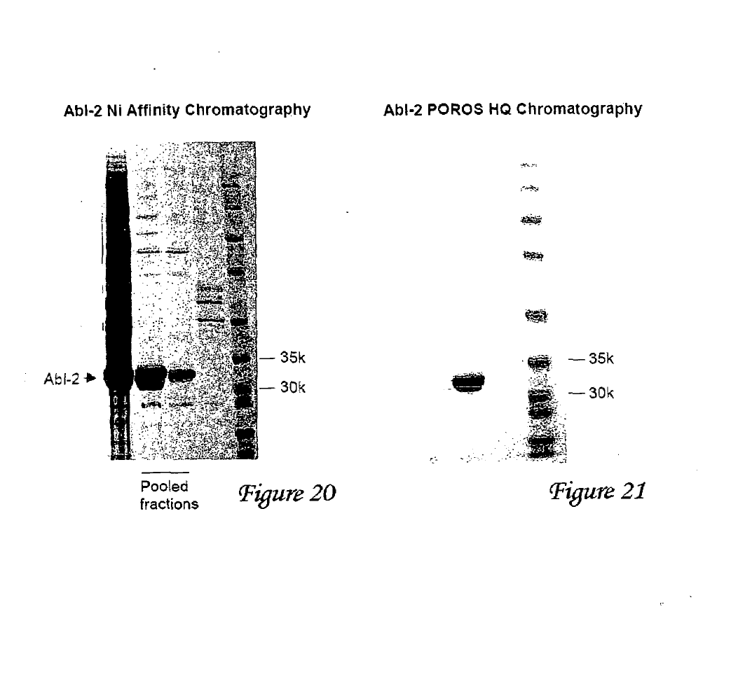

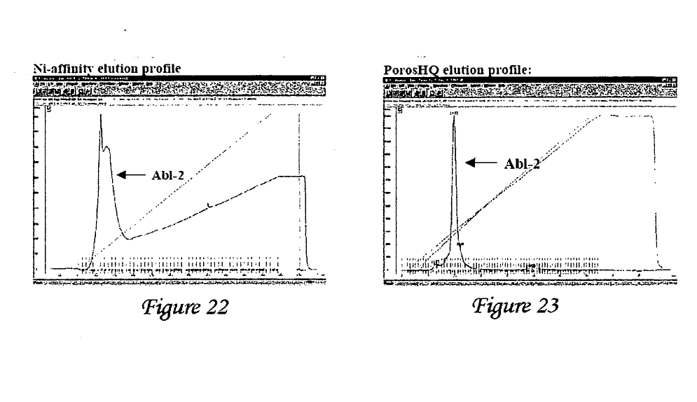

FIGS. 20 and 21 illustrate the purity of Abl-construct 2 expressed in the presence of PD-180970 after Nickel affinity chromatography (FIG. 20) and subsequent POROS HQ anion exchange chromatography (FIG. 21). FIG. 22 shows the elution profile for Abl construct 2 from Nickel affinity chromatography, and FIG. 23 depicts the elution profile for Abl construct 2 from POROS HQ anion exchange chromatography. This form of Abl is in its unphosphorylated physical state.

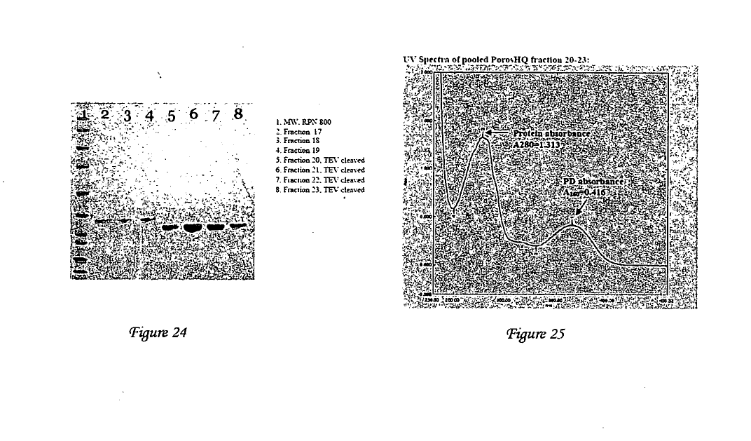

FIG. 24 illustrates the elution profile of Abl kinase having SEQ ID. NO. 53 after treatment with tev protease to remove the Glu-6×His-TEV affinity tag. Fractions 17-19 contain Abl protein with the Glu-6×His-TEV tag still intact, while fractions 20-23 contain Abl protein wherein the Glu-6×His-TEV tag has been removed. UV analysis (FIG. 25) of the pooled fractions 20-23 revealed an absorbance maximum at 360 nm indicative of the presence of the ATP competitive inhibitor PD 180970 still bound to the Abl ATP pocket, thus ensuring the preservation of Abl protein in its unphosphorylated state during expression and purification.

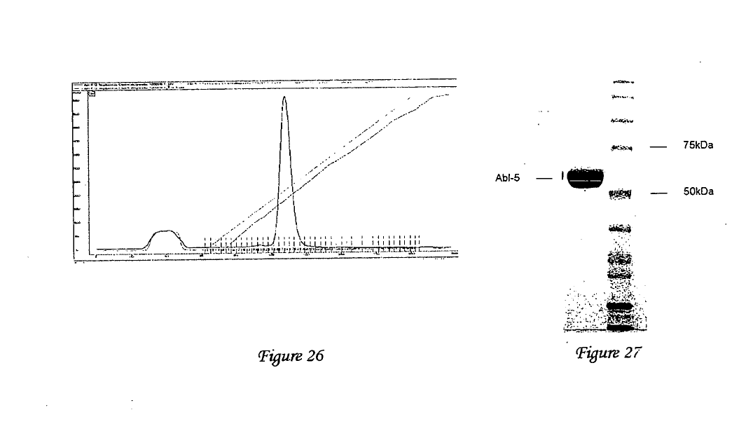

FIG. 26 illustrates the elution profile of Abl kinase having SEQ ID. NO. 51 upon purification through Nickel affinity chromatography and Q-Sepharose chromatography. FIG. 27 illustrates SDS-Page analysis of purified pooled fractions.

p38-alpha Kinase

Whole gene synthesis of p38-alpha kinase having SEQ ID NO. 48 (6×His-TEV tag, full length) was completed and proteins were expressed in E. coli using both arabinose-inducible and T7 promoter vectors. The expression of p38-alpha kinase in two expression vectors (pET15b and pBAD) was examined after induction with 0.5 M IPTG (pET15b) or 0.2% arabinose (pBAD). Protein expression was determined by immunoprecipitation and SDS-Page. Expression of p38-alpha in pBAD constructs after induction was clearly demonstrable in immunoprecipitates with ant-GLU monoclonal antibodies.

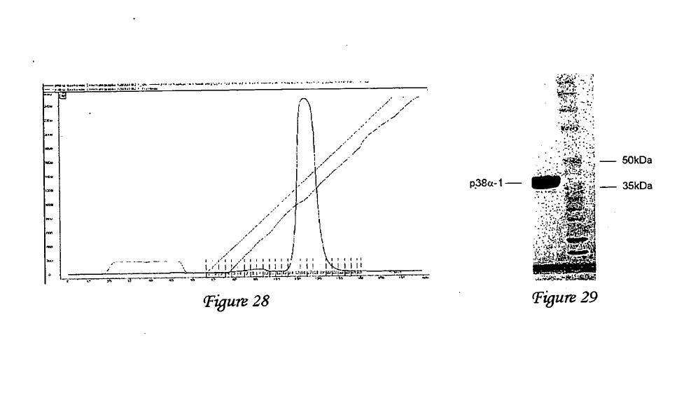

FIG. 28 illustrates the elution profile of p38-alpha protein upon Q-Sepharose chromatography. An SDS-Page of pooled purified fractions is illustrated in FIG. 29.

### Braf Kinase

Whole gene synthesis was completed on Seq ID NO. 61 (Braf kinase 4, 6×His-tag, residues 432-723 of the full length sequence and oncogenic V599E Braf kinase with Seq ID NO. 58 (6×His-tag, residues, V599E, 432-723 of the full length sequence. Baculovirus transfection cultures (40 mL of infected Sf9, 20 ml of infected Sf9-Pro and control-infected Sf9) were harvested at 2 days and lysed in 2 mL NP40 lysis buffer. Ni-chelating pull-downs and KT3 immuno precipitations were done with 1.5 mL and 0.2 mL of lysate respectively (8 uL of bead bed volume). Protein bound to the beads was eluted with SDS-sample buffer and run on an SDS-PAGE gel. Purification of 60 grams of cell paste yielded 4.5 mg of highly purified oncogenic V599E Braf kinase using the following five step procedure:

1. Ni Chromatography I: The first step in Braf purification utilized the engineered His-tag and isolation of the ternary complex (Braf/p50cdc37/Hsp90) on a Ni column.

2. POROS HS Chromatography: The pooled Ni fractions were then run through the POROS HS cation-exchange column where >90% of the ternary complex flows through.

3. Ni Chromatography II: The ternary complex was disassociated by a second passage through the Ni column. The disassociation was only partially complete and thus resulted in a main ternary complex peak and a second Braf peak containing one lower MW contaminant.

4. Ni Chromatography III: Due to the inefficient disassociation of the ternary complex after the second Ni column the ternary complex was again separated on the Ni column. This third Ni chromatography step resulted in a further disassociation of the complex and greater Braf yields.

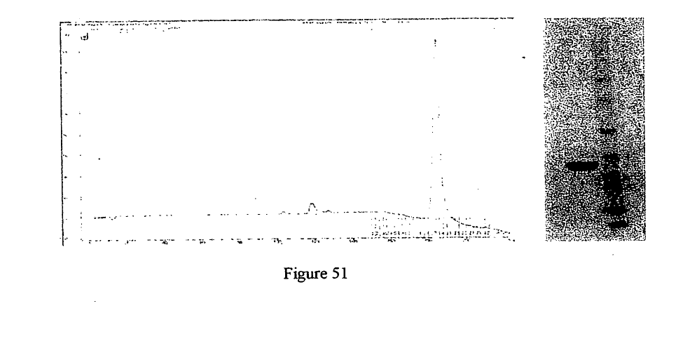

5. Heparin Chromatography: The final step in Braf purification utilized heparin chromatography, which separated Braf from the lower MW contaminant and effectively concentrated the pooled Ni fractions. An SDS-PAGE of the purified Braf is shown in FIG. 51.

### Gsk-3 Beta Kinase

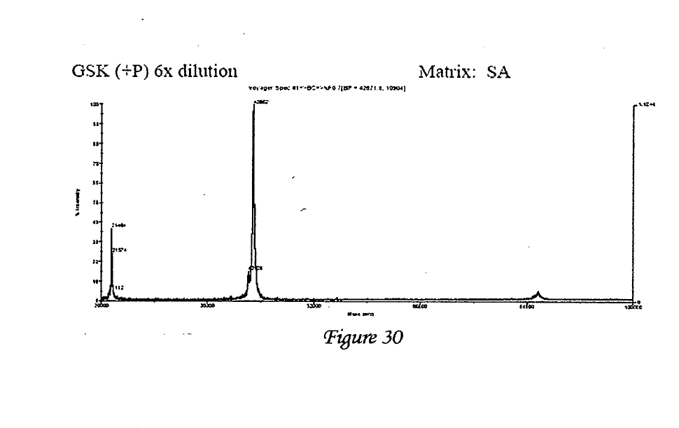

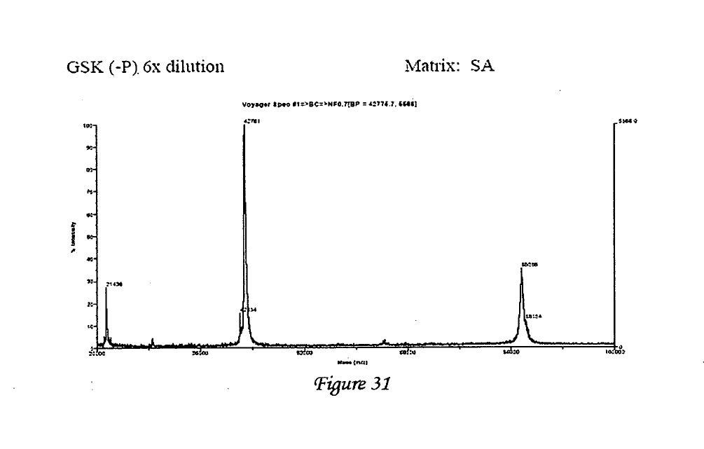

Whole gene synthesis was completed on kinase having SEQ ID NO. 54 (6×His-TEV tag, full length), kinase having SEQ ID NO. 49 (10×His, residues 27-393), and kinase having SEQ ID NO. 50 (Glu-6×His-TEV tag, residues 35-385). Transfections were performed in insect cells. Protein expression was determined by immuno precipitation and SDS-Page. The expression level for construct 3 exceeded 5 mg/L. Purification of gsk-3 beta protein involved procedures that allowed isolation of both switch control ligand unphosphorylated kinase (GSK−P) and switch control ligand phosphorylated kinase (GSK+P) forms from the same expression run. Nickel affinity chromatography was performed in 20 mM HEPES buffer at pH7.5. This step was followed by POROS HS (cation-exchange) chromatography. FIG. 30 illustrates the MALDI-TOF spectrum of the GSK+P protein indicating the expected molecular ion of 42862 Da. FIG. 31 illustrates the MADLI-TOF spectrum of the GSK−P protein indicating the expected molecular ion of 42781.

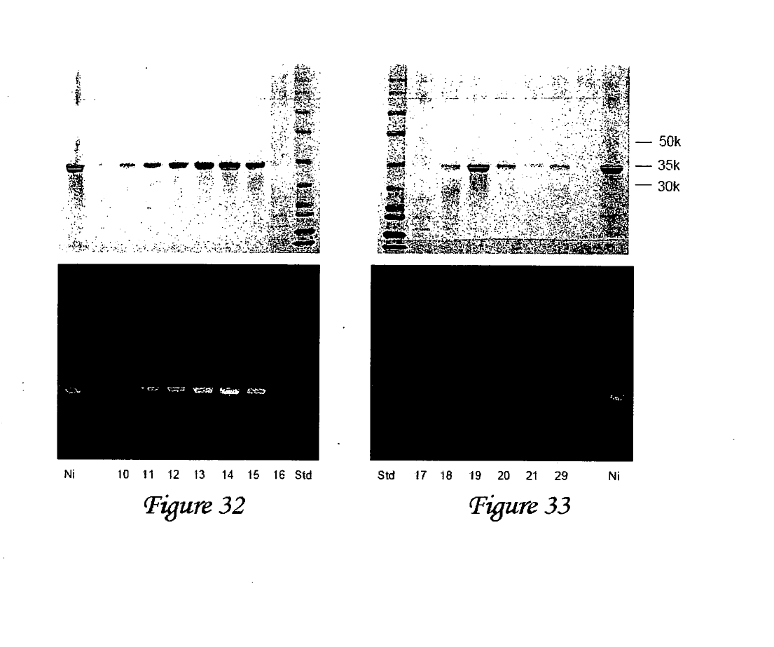

FIGS. 32 and 33 illustrate analysis of POROS HS chromatography fractions by SDS-PAGE analysis in conjunction with staining by the antiphosphotyrosine antibody PY-20. Fractions 10-15 were imaged by the PY-20 antibody, indicating the presence of phosphate on the switch control ligand tyrosine residue. Fractions 17-29 were not imaged by the PY-20 antibody, indicating the absence of switch control ligand phosphorylation of tyrosine.

### Example C

### Step 5. Screening of the Purified Proteins with Candidate Small Molecule Switch Control Modulators

Fluorescence Affinity Assay for Probing the Binding of Switch Control Inhibitors into Kinase Protein Switch Control Pockets

A fluoroprobe which does not fluoresce unless it is bound into the ATP pocket of a kinase is utilized in a general way to establish a fluorescence affinity assay. This fluorescence affinity assay is utilized in an affinity-based screen to identify small molecules which bind into switch control pockets of protein kinases. Binding of small molecule switch inhibitors displaces the switch control ligand phenylalanine of the DFG motif into an orientation which sterically blocks the ATP pocket. Such inhibitor-induced blockade of the ATP pocket is registered as an inhibition of binding of the fluoroprobe into the ATP pocket.

SKF 86002 is the fluoroprobe used in the p38 kinase fluorescence affinity assay. This fluoroprobe has been previously described (C. Pargellis, et al., Nature Structural Biology (2002) 9, 268-272; J. Regan, et al, J. Med. Chem. (2002) 45, 2994-3008). PD 166326 is utilized as the fluoroprobe in the fluorescence affinity assays for Abl kinase and Braf kinase. The structures of SKF 86002 and PD 166326 are shown below. PD 166326 has been previously reported as an ATP competitive protein kinase inhibitor (D. R. Huron et al, Clinical Cancer Res. (2003) 9: 1267).

As a matter of experimentation, other fluoroprobes for these and other kinases can be identified by i) identifying ATP-competitive inhibitors with potencies in the range of 1.0-10,000 nM, preferably 10-1,000 nM; ii) determining that the candidate fluoroprobe does not inordinately fluorescence in the absence of the candidate kinase; iii) determining that the candidate kinase does not inordinately fluorescence in the absence of the candidate fluoroprobe; iv) determining that measurable fluorescence is observed upon combining both the candidate fluoroprobe and the candidate kinase in the same experiment; v) determining that candidate small molecule switch control inhibitors can modulate the fluorescence of the fluoroprobe upon co-incubation. Examples of such candidate fluoroprobes for c-Abl or Bcr-Abl kinases can, by way of illustration, be taken from disclosed ATP-competitive inhibitors: see J. Wissing et al, Molecular and Cellular Proteomics (2004) 3: 1181; N. P. Shah et al, Science (2004) 305: 399).

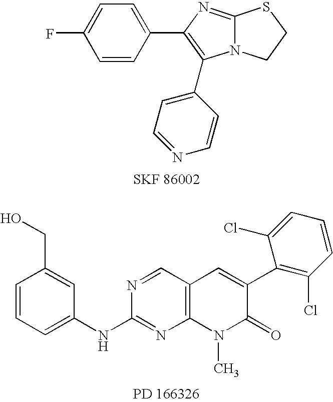

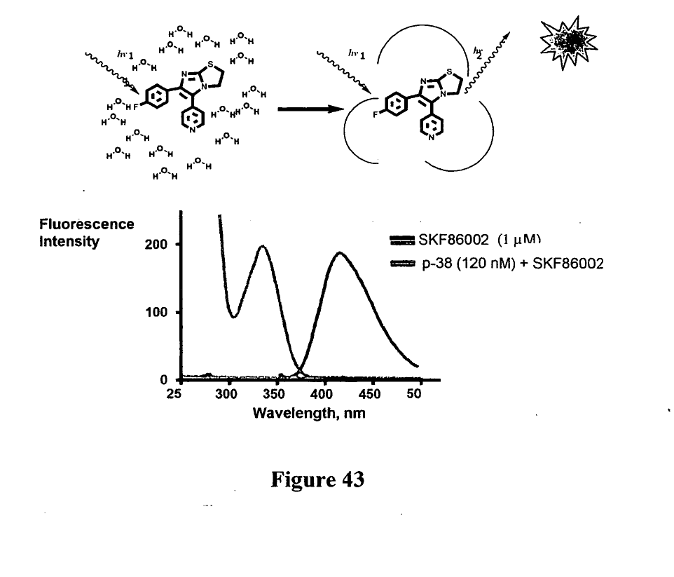

FIG. 43 illustrates the excitation absorbance spectrum and the fluorescence emission spectrum of SKF 86002 when bound into the ATP pocket of p38-alpha kinase. When either the fluoroprobe SKF 86002 or p38-alpha kinase was evaluated alone, there was no significant excitation or emission spectrum observed. Only when SKF 86002 and p38-alpha kinase were evaluated in combination was the excitation and emission spectrum of SKF 86002 realized.

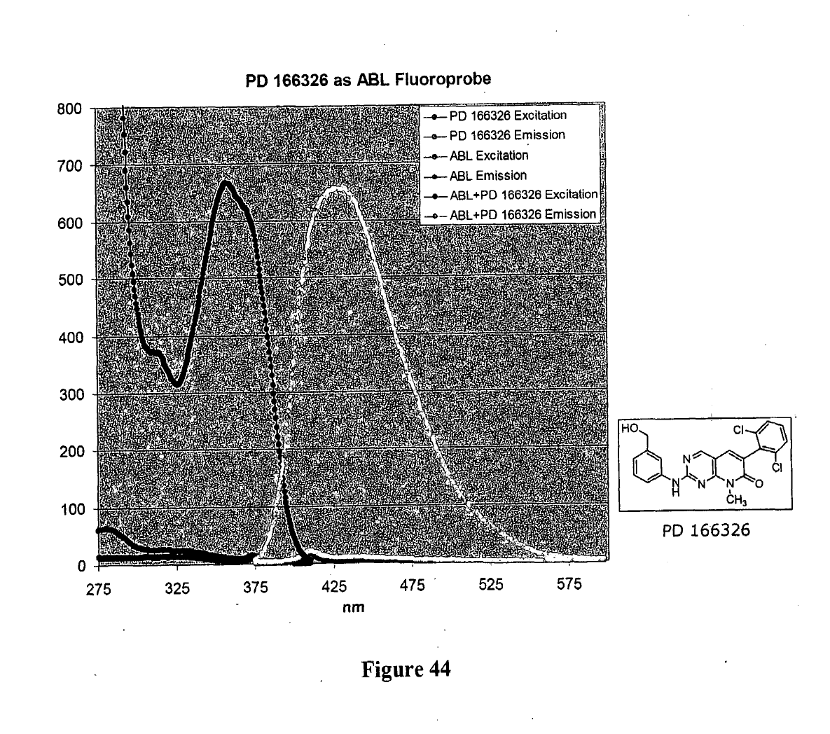

FIG. 44 illustrates the excitation absorbance spectrum and the fluorescence emission spectrum of PD 166326 when bound into the ATP pocket of Abl kinase. When either the fluoroprobe PD 166326 or Abl kinase was evaluated alone, there was no significant excitation or emission spectrum observed. Only when PD 166326 and Abl kinase were evaluated in combination was the excitation and emission spectrum of PD 166326 realized.

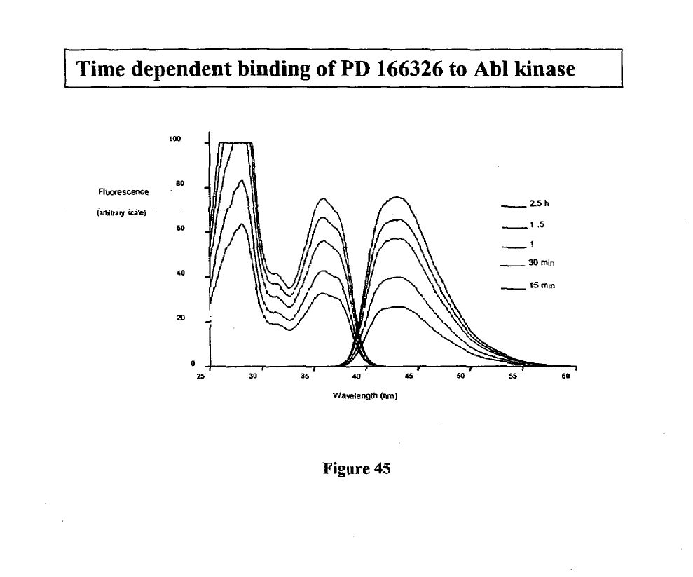

PD 166326 is a slow binding fluoroprobe to Abl kinase. FIG. 45 illustrates the time course of the excitation-emission spectra. Using a ratio of 100 nM PD 166326 to 40 nM Abl kinase, 90 minutes was required at 30° C. to establish the maximal excitation-emission spectra. Therefore, PD 166326 is a slow binding fluoroprobe, requiring flexibility by Abl kinase in order to achieve maximal binding into the ATP pocket of Abl kinase.

Fluorescence Affinity Assay Protocol for Unphospho-p38-alpha or Phospho-p38-alpha Kinase

### A. Materials

1. p38-alpha kinase
   a. Phospho-p38-alpha kinase from Roche Applied Diagnostics (0.8 mg/ml (12.5 uM))
   b. Unphospho-p38-alpha kinase from Decode Genetics, Inc. (0.52 mg/ml, 12 uM)
2. SKF86002 fluoroprobe (EMD Biosciences)
3. Bis-Tris propane buffer (20 mM, pH 7) with 0.15% n-octyl-glucoside
4. 384 microplate (Greiner 781091, Nuclear)
5. Polarstar Optima plate reader (BMG)

### B. Procedure:

1. Make Solution A containing 48 nM p38 and 2 uM SKF86002 in Bis-Tris buffer
2. Serially dilute test compounds in DMSO and in the reaction buffer according to Step 1 and 2 of Table 22 below. This dilution can be done automatically on a Tecan (or similar) robotic workstation or performed manually.
3. Mix Solution A with the diluted compound solutions following Step 3 of Table 22. This step can be performed automatically on a Tecan (or similar) robotic workstation or performed manually.
4. Incubate at 30° C. or room temp for 2 h, depending on protein stability.
5. Read at emission wavelength 420 nm upon excitation at 340 nm.

TABLE 22
Scheme for compound serial dilution and mixing compound with enzyme and SKF86002

| | | Well | | | | | | | | | |
|---|---|---|---|---|---|---|---|---|---|---|---|
| Step | | 1 | 2 | 3 | 4 | 5 | 6 | 7 | 8 | 9 | 10 |
| 1 | Plate 1 (96 well) | | | | | | | | | | |
| | Add 10 mM stock | 2 uL* | | | | | | | | | |
| | Add 100% DMSO | 198 ulL | 1:1 serial dilution (100 uL at each well) | | | | | | | | |
| | Inhibitor, uM | 100 | 50 | 25 | 12.5 | 6.25 | 3.125 | 1.5625 | 0.78125 | 0.39063 | 0.19531 |
| 2 | Plate 2 (96 well) | | | | | | | | | | |
| | Add the buffer with | 245 uL | 245 uL | 245 uL | 245 uL | 245 uL | 245 uL | 245 uL | 245 uL | 245 uL | 245 uL |
| | 5% DMSO | | | | | | | | | | |
| | Remove from plate 1 | 5 uL | 5 uL | 5 uL | 5 uL | 5 uL | 5 uL | 5 uL | 5 uL | 5 uL | 5 uL |
| | Inhibitor, uM | 2 | 1 | 0.5 | 0.25 | 0.125 | 0.0625 | 0.03125 | 0.01563 | 0.00781 | 0.00391 |
| 3 | Plate 3 (384 well | | | | | | | | | | |
| | Rxn plate) | | | | | | | | | | |
| | Remove from plate | 50 | 50 | 50 | 50 | 50 | 50 | 50 | 50 | 50 | 50 |
| | 2, ul | | | | | | | | | | |
| | Add Solu A, uL | 50 | 50 | 50 | 50 | 50 | 50 | 50 | 50 | 50 | 50 |
| | Final inhibitor, uM | 1.0 | 0.50 | 0.25 | 0.125 | 0.0625 | 0.0313 | 0.0156 | 0.0078 | 0.0039 | 0.0020 |

*The amount used in Step 1 is determined by the highest desired final compound concentration. In this procedure, the highest screening compound is 1 uM (see Step 3, “Final inhibitor, uM”). If the highest screening concentration is to be 100 nM, then 2 ul of 1 mM stock will be used in Step 1.

### C. Protocol and Results.

The assay was performed in a 384 plate (Greiner Nuclear 384 plate) on a Polarstar Optima plate reader (BMG). Typically, the reaction mixture contained 1 uM SKF 86002, 80 nM p38-alpha kinase, and various concentrations of an inhibitor in 20 mM Bis-Tris Propane buffer, pH 7, containing 0.15% (w/v) n-octylglucoside and 2 mM EDTA in a final volume of 65 uL. The reaction was initiated by addition of the enzyme. The plate was incubated at room temperature (˜25° C.) for 2 hours before reading the emission at 420 nm upon excitation at 340 nm. By comparison of RFU (relative fluorescence units) values with that of a control (in the absence of small molecule modulators), the percentage of inhibition at each concentration of the small molecules was calculated. IC₅₀ values for the small molecule modulators were calculated from the % inhibition values obtained at a range of concentrations of the small molecule modulators using Prism (available from GraphPad, Inc.). When time-dependent inhibition was assessed, the plate was read at multiple reaction times such as 0.5, 1, 2, 3, 4 and 6 hours. The IC₅₀ values were calculated at each time point. An inhibition was assigned as time-dependent if the IC₅₀ values decrease with the reaction time (more than two-fold in four hours). IC₅₀ values of representative small molecules are shown in Table 23.

TABLE 23

| | | Time- |
|---|---|---|
| Example # | IC50, nM | dependent |
| 1 | 292 | Yes |
| 2 | 997 | No |
| 3 | 231 | Yes |
| 4 | 57 | Yes |
| 5 | 1107 | No |
| 6 | 238 | Yes |
| 7 | 80 | Yes |
| 8 | 66 | Yes |
| 9 | 859 | No |
| 10 | 2800 | No |
| 11 | 2153 | No |
| 12 | ~10000 | No |
| 13 | 384 | Yes |
| 15 | 949 | No |
| 19 | ~10000 | No |
| 21 | 48 | Yes |
| 22 | 666 | No |
| 25 | 151 | Yes |
| 26 | 68 | Yes |
| 29 | 45 | Yes |
| 30 | 87 | Yes |
| 31 | 50 | Yes |
| 32 | 113 | Yes |
| 37 | 497 | No |
| 38 | 508 | No |
| 41 | 75 | Yes |
| 42 | 373 | No |
| 43 | 642 | No |
| 45 | 1855 | No |
| 46 | 1741 | No |
| 47 | 2458 | No |
| 48 | 3300 | No |
| 57 | 239 | Yes |

IC₅₀ values obtained at 2 hours reaction time

Further evaluations of small molecule switch control inhibitors in the fluorescence affinity assay for p38-alpha kinase are shown in Table 24. In these cases, the p38-alpha kinase concentration was lowered to 24 nM. Small molecule switch inhibitors were able to bind to switch control pockets in either unphosphorylated p38-alpha kinase (column 2) or doubly phosphorylated p38-alpha kinase (phosphotreonine180+phosphotyrosine182, column 3). The switch control inhibitors exhibited similar potencies for displacement of fluoroprobe SKF86002 regardless of the phosphorylation state of p38-alpha kinase. Specifically, switch inhibitors were able to induce the switch control ligand to adopt its off switch state in both unphosphorylated p38-alpha kinase, wherein the switch is inherently predisposed to predominate in the off switch state, and in doubly phosphorylated p38-alpha kinase, wherein the switch is inherently predisposed to predominate in the on switch state. Column 4 illustrates the relative potency of small molecule switch inhibitors when evaluated with unphosphorylated p38-alpha kinase or doubly phosphorylated p38-alpha kinase. A ratio of 1.0 indicates equal potency of the inhibitor for both forms of p38-alpha kinase. A ratio greater than 1.0 indicates a preference for inhibiting the unphosphorylated form of p38-alpha kinase relative to the doubly phosphorylated form. A ratio less than 1.0 indicates a preference for inhibiting the doubly phosphorylated form of p38-alpha kinase relative to the unphosphorylated form.

TABLE 24

| | FP IC50 | FP IC50 | |
|---|---|---|---|
| | U-p38-alpha | P-p38-alpha | Ratio IC50s |
| Example 29 | 0.016 | 0.056 | 3.5 |
| Example 61 | 0.024 | 0.019 | 0.8 |
| Example 63 | 0.028 | 0.085 | 0.8 |
| Example 69 | 0.009 | 0.011 | 1.2 |
| Example 70 | 0.026 | 0.026 | 2.3 |
| Example 71 | 0.017 | 0.019 | 1.1 |
| Example 72 | 0.040 | 0.067 | 1.7 |
| Example 75 | 0.165 | 0.119 | 0.7 |
| Example 76 | 0.028 | 0.026 | 0.9 |
| Example 77 | 0.025 | 0.057 | 1.1 |
| Example 78 | 0.233 | 0.195 | 1.5 |
| Example 92 | 0.052 | 0.160 | 3.1 |
| Example 93 | 0.016 | 0.039 | 2.4 |
| Example 94 | 0.010 | 0.019 | 1.9 |
| Example 95 | 0.020 | 0.030 | 1.5 |
| Example 96 | 0.007 | 0.008 | 3.0 |

~24 nM enzyme; 1.0 uM SKF-86002
IC₅₀ values in uM

### Fluorescence Affinity Assay for Abl Kinase

A. Materials
1. c-Abl kinase (SEQ ID NO. 51), 0.24 mg/ml, 7.5 uM
2. PD 166326
3. Tris buffer: 90 mM Tris-HCl buffer, pH 7.5, containing 0.2% octyl-glucoside
4. 384 microplate (Greiner 781091, Nuclear)
5. Polarstar Optima plate reader (BMG) or equivalent

B. Procedure:
1. Make Solution A containing 80 nM Abl in the Tris buffer
2. Serially dilute test compounds in Solution A (50 uL per well)
3. Incubate the plate at room temp for 0.5 h
4. Prepare 200 nM PD 166326 with the Tris buffer
5. Add 50 uL of the PD 166326 solution into each well containing Solution A
6. Read immediately at an emission wavelength of 460 nm upon excitation at 355 nm
7. Include positive controls (wells containing no inhibitor) and background controls (wells containing PD 166326 only) in each run

### C. Protocol and Results

The assay was performed in a 384 plate (Greiner Nuclear 384 plate) on a Polarstar Optima plate reader (BMG). Typically, the reaction mixture contained 100 nM PD 166326, 40 nM Abl kinase, and various concentrations of an inhibitor in 20 mM Bis-Tris Propane buffer, pH 7, containing 0.15% (w/v) n-octylglucoside and 2 mM EDTA in a final volume of 65 uL. Abl kinase was preincubated with test compounds for 2 h at 30° C. in the absence of fluoroprobe. The reaction was initiated by addition of PD 166326. The plate was incubated at room temperature (˜30° C.) for 2 hours before reading the emission at 460 nm upon excitation at 355 nm. By comparison of RFU (relative fluorescence units) values with that of a control (in the absence of small molecule modulators), the percentage of modulation at each concentration of the small molecules was calculated.

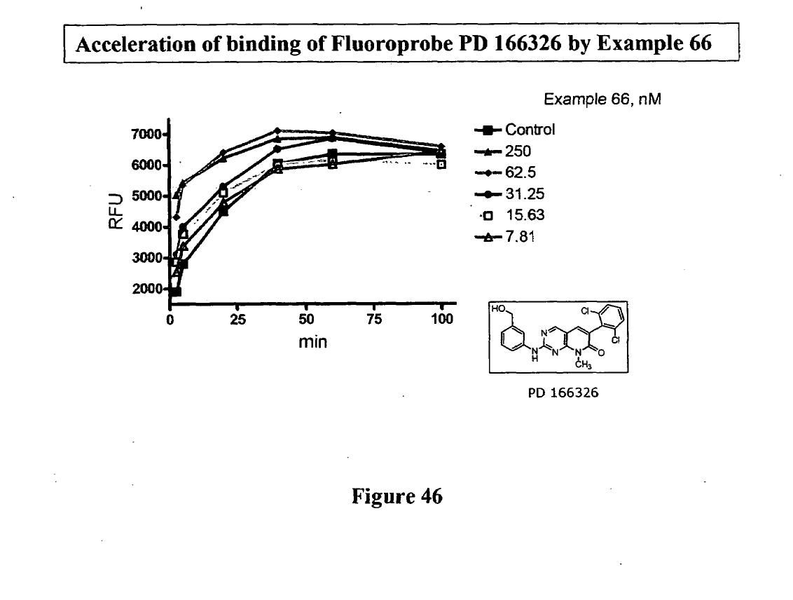

Examples of evaluations of small molecule switch control inhibitors in the fluorescence affinity assay for Abl kinase are shown in FIG. 46. Unexpectedly, small molecule switch control inhibitors of Abl kinase did not displace binding of the ATP-competitive fluoroprobe PD 166326; rather small molecule inhibitors accelerated and stabilized the binding of PD 166326. By way of exemplification, the switch inhibitor of Example 66 caused a concentration-dependent acceleration of the binding of fluoroprobe PD 166326. The final relative fluorescence units (RFU) of the bound PD 166326 was the same value of ˜6000 RFU, indicating that at this level of RFU the ATP pocket of Abl kinase was saturated with the fluoroprobe PD 166326.

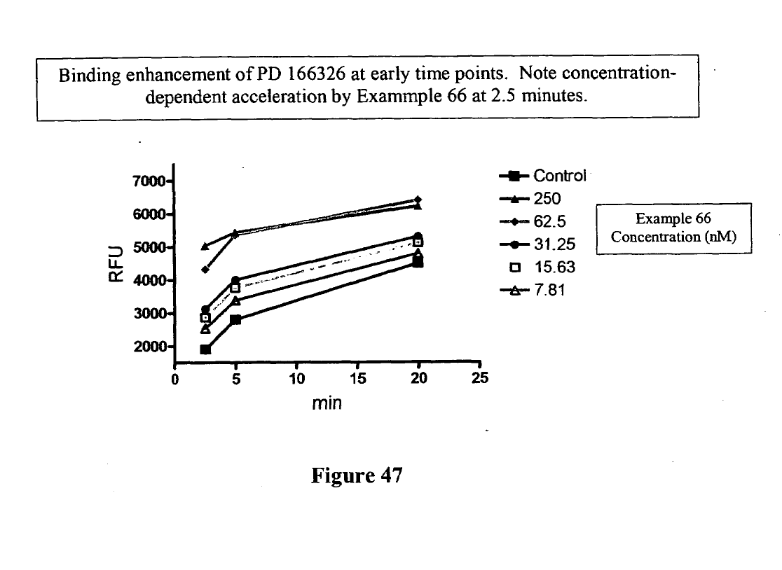

FIG. 47 is an expanded graph which more clearly illustrates the concentration-dependent acceleration of binding of the fluorprobe PD 166326 to the ATP pocket of Abl kinase caused by coincubation with switch inhibitor Example 66.

The co-crystal structure of Example 65 bound to Abl kinase was determined (vida infra). Example 65 occupies the on composite switch control pocket of Abl kinase, wherein the phenylalanine 401 from the DFG motif is in the ‘out’ conformation. The co-crystal structure of PD 166326 bound to Abl kinase has also been determined (B. Nagar et al, Cell (2003) 112: 859). PD 166326 binds into the ATP pocket of Abl kinase. The mode of binding of PD 166326 also displaces phenylalanine 401 of the DFG motif into the “out” conformation. The acceleration of binding of fluoroprobe PD 166326 to Abl kinase by switch control inhibitor Example 65 and related analogs is explained by the binding of switch control inhibitor into the switch control pocket of Abl kinase, inducing phenylalanine 401 into the ‘out’ conformation. This induced conformational change caused by Example 65 places Abl kinase in a conformation that is more conducive to binding of the ATP competitive inhibitor PD 166326. Switch inhibitors of kinases can coexist with and synergize the binding of ATP competitive inhibitors when said ATP competitive inhibitors bind with the phenylalanine of the DFG motif in the ‘out’ conformation. Such synergy and/or mutual binding of both classes of inhibitors to a kinase find utility in the treatment of mammalian diseases wherein there is a need to inhibit a protein kinase by more than one mechanism, including the need to utilize cocktail drug treatments to keep selective pressure of a cancer-causing kinase such as Bcr-abl kinase from developing resistance to a single drug agent.

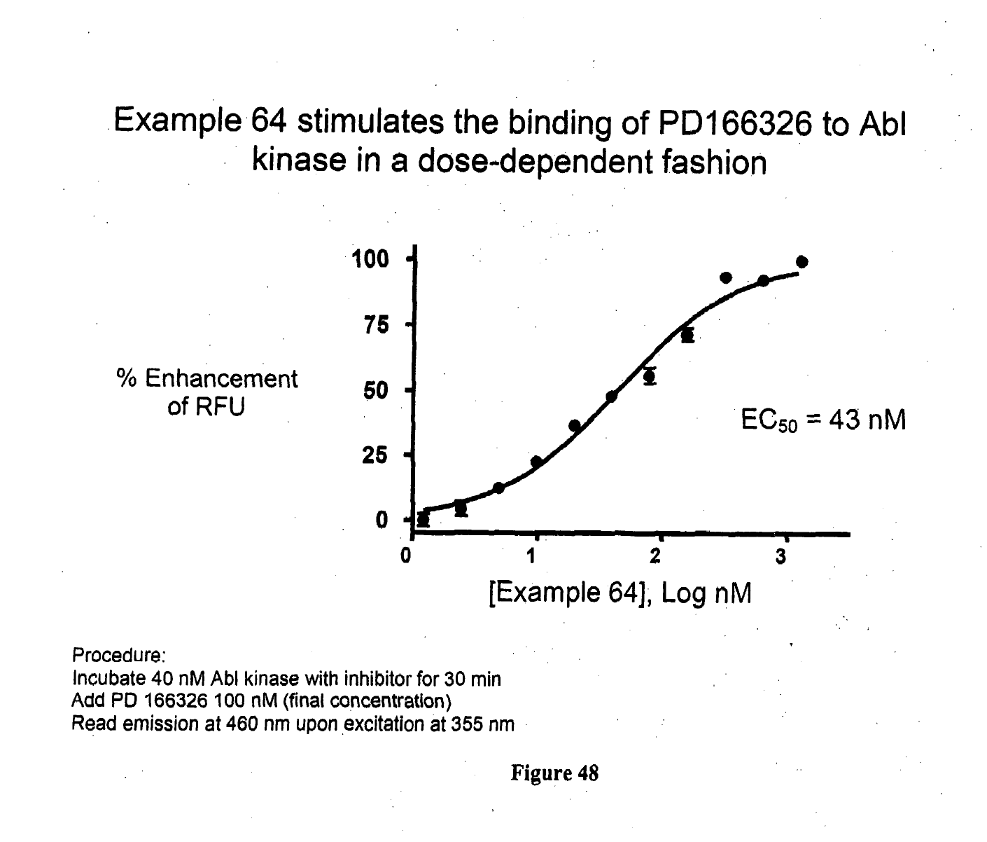

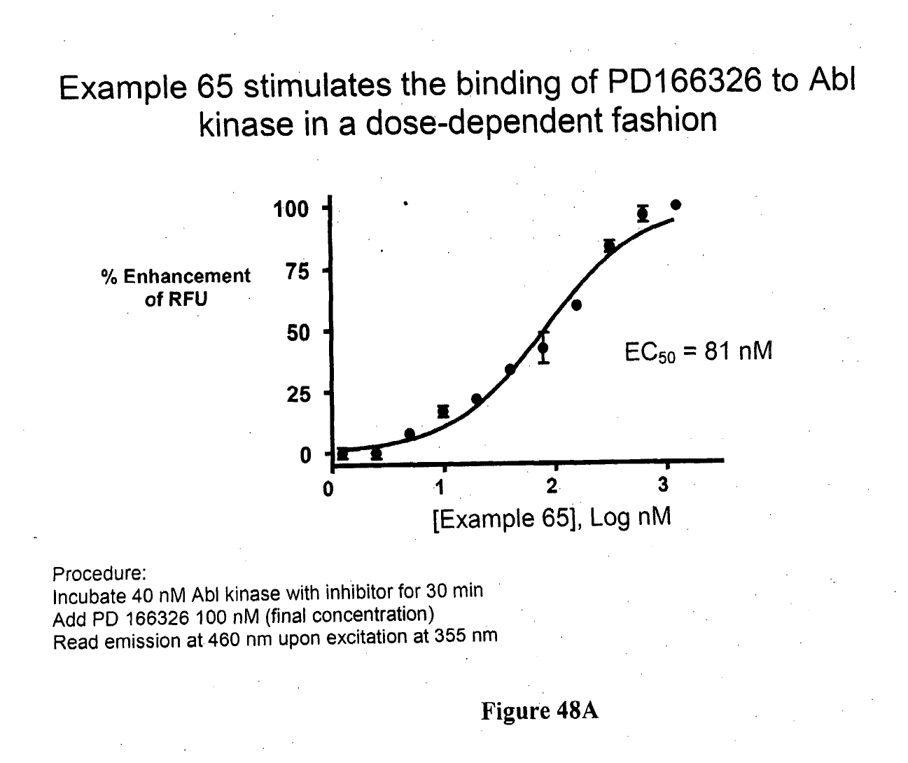

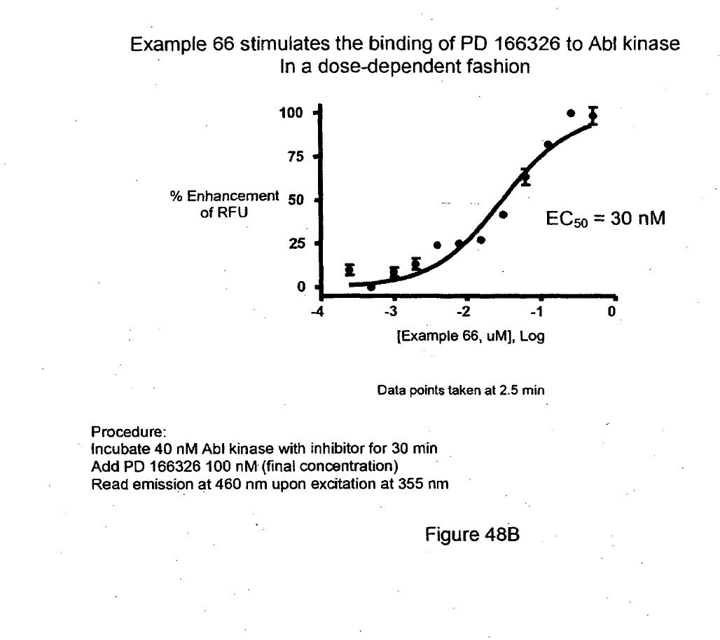

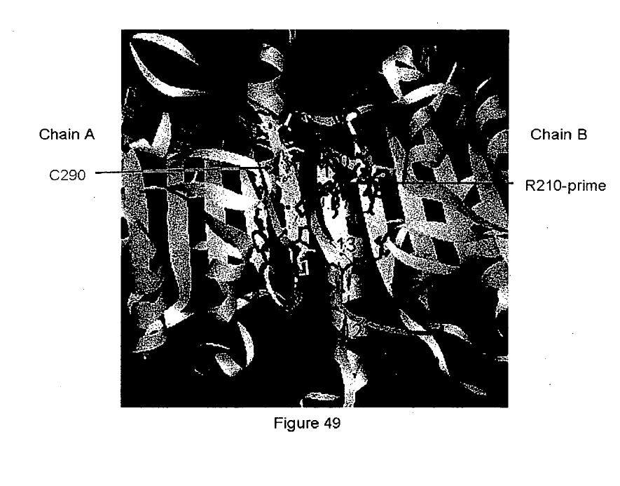

Since the effect of a switch control inhibitor to accelerate the binding of the ATP-competitive fluoroprobe PD 166326 was saturable, an EC₅₀ value for affecting the accelerated binding was determined to quantify the potency of the switch inhibitors. FIG. 48 illustrates the saturable curve of Example 64 for accelerating the early time-point binding of fluoroprobe PD 166326 to c-Abl kinase. An EC₅₀ value of 0.043 uM was experimentally derived for Example 64 in its effect to accelerate the binding of fluoroprobe PD 166326. FIG. 48A illustrates the saturable curve of Example 65 for accelerating the early time-point binding of fluoroprobe PD 166326 to c-Abl kinase. An EC₅₀ value of 0.081 uM was experimentally derived for Example 65 in its effect to accelerate the binding of fluoroprobe PD 166326. FIG. 48B illustrates the saturable curve of Example 66 for accelerating the binding of fluoroprobe PD 166326 to c-Abl kinase. An EC₅₀ value of 0.030 uM was experimentally derived for Example 66 in its effect to accelerate the binding of fluoroprobe PD 166326.

Thermal Denaturation of Unphosphorylated and Phosphorylated p38-alpha Kinases

The binding of small molecules into unphosphorylated and phosphorylated p38-alpha kinases was demonstrated by thermal denaturation (thermal melt, TM) that compares the temperature at which the protein in solution in the presence of a small molecule modulator becomes denatured (melts) compared to the melt temperature of the protein in solution alone (apo protein). Denaturation was monitored by measuring the absolute absorbance of the solutions at an appropriate wavelength (typically 230 nm and 240 nm) as a function of temperature. As the protein became denatured, additional UV absorbing functional groups became exposed causing an increase in absorbance at the melting temperature. Increased compound affinity was marked by a shift in the melting temperature to higher values. The thermal data were collected using Agilent spectrophotometers and ChemStation UV/Vis Thermal Denaturation software.

Test solutions containing both small molecule and unphosphorylated p38-alpha kinase were prepared as follows: 1.5 μL of a 10 mM inhibition compound in DMSO was mixed with 997 μL Buffer (Bis-Tis Propane 19 mM pH7, NaCl 86.5 mM, EDTA 1.73 mM, 0.15% (w/v) octyl β-D-glucopyranoside (OG), 3.5% (v/v) DMSO) then 1.5 μL of 184 μM p38-alpha kinase was added to the solution. The final concentration was 0.28 μM p38-alpha kinase and 15 μM small molecule. The final protein and buffer solution was mixed gently using a pipetter to minimize denaturing due to over aggressive mixing. The final solution was transferred to a 50 μL cuvette for analysis.

Test solutions containing only unphosphorylated p38-alpha kinase were prepared as follows: 1.5 μL of 184 μM p38-alpha kinase was mixed with 998 μL Buffer to give a concentration of 0.28 μM p38 in Buffer. The final solution was transferred to a 50 μL cuvette for analysis.

Test solutions containing both small molecule and doubly phosphorylated p38-alpha kinase were prepared as follows: 2 μL of a 10 mM small molecule solution in DMSO was mixed with 998 μL Buffer (Bis-Tis Propane 19 mM pH7, NaCl 86.5 mM, EDTA 1.73 mM, 0.15% (w/v) octyl β-D-glucopyranoside (OG), 3.5% (v/v) DMSO) to give a concentration of 20 μM small molecule in buffer. 2 μL of 25 μM p38-alpha kinase was mixed with 98 μL of the small molecule/buffer solution. The final concentration was 0.5 μM p38-alpha kinase and 20 μM small molecule. The final protein and buffer solution was mixed gently using a pipetter to minimize denaturing due to over aggressive mixing. The final solution was transferred to a 50 μL cuvette for analysis.

Test solutions containing only doubly phosphorylated p38-alpha kinase were prepared as follows: 2 μL of 25 μM p38-alpha kinase was mixed with 98 μL Buffer to give a concentration of 0.5 μM pP-38 in Buffer. The protein and buffer solution was mixed gently using a pipetter to minimize denaturing due to over aggressive mixing. The final solution was transferred to a 50 μL cuvette for analysis.

UV analysis was performed on an Agilent 8453 or 8452A UV/Vis spectrophotometer equipped with a Peltier temperature controller. The temperature profile increased from 25° C. to 70° C. in 0.2° C. increments over 8 hours with UV monitoring at 240 nm. A first derivative calculation with data smoothing was applied to the resulting data to report the point of inflection as the melting temperature.

As shown in Table 25, binding of a switch control inhibitor to unphosphorylated or doubly phosphorylated p38-alpha kinase resulted in a thermal stabilization of p38-alpha kinase relative to the apo kinase controls. In general, the thermal stabilization of unphosphorylated p38-alpha kinase was higher than phosphorylated p38-alpha kinase.

TABLE 25

| | ΔTm (° C.) relative | |
|---|---|---|
| | to | ΔTm (° C.) relative |
| | unphosphorylated | to doubly |
| Example | apo p38 | phosphorylated p38 |
| 29 | 10.1 | 7.3 |
| 61 | 8.5 | 5.9 |
| 63 | 9.4 | 5.0 |
| 67 | 9.5 | |
| 68 | 10.1 | |
| 69 | 13.1 | 9.1 |
| 70 | 12.6 | 8.7 |
| 71 | 10.9 | 9.6 |
| 72 | 11.3 | 8.1 |
| 73 | 8.9 | 5.9 |
| 74 | 8.4 | 7.3 |
| 75 | 8.2 | 5.6 |
| 76 | 13.1 | 9.4 |
| 77 | 8.0 | 5.3 |
| 78 | 6.7 | 3.9 |

### Thermal Denaturation of Oncogenic V599E Braf Kinase

The binding of switch control inhibitors into V599E Braf kinase was demonstrated using thermal denaturation by comparing the temperature at which the protein in solution with the small molecule becomes denatured (melts) compared to the melt temperature of the protein in solution alone (apo protein). Denaturation was monitored by measuring the absolute absorbance of the solutions at an appropriate wavelength (typically 230 nm and 240 nm) as a function of temperature. As the protein became denatured, additional UV absorbing functional groups became exposed causing an increase in absorbance at the melting temperature. Increased compound affinity was marked by a shift in the melting temperature to higher values. The thermal data were collected using Agilent spectrophotometers and ChemStation UV/Vis Thermal Denaturation software.

Test solutions containing both small molecule and V599E Braf kinase were prepared as follows: 2 μL of a 10 mM small molecule in DMSO solution was mixed with 498 μL Buffer (Bis-Tis Propane 19 mM pH7, NaCl 86.5 mM, EDTA 1.73 mM, 0.15% (w/v) octyl β-D-glucopyranoside (OG), 3.5% (v/v) DMSO) to give a concentration of 40 μM small molecule in buffer. 50 μL of 2.8 μM V599E Braf kinase was mixed with 50 μL of the small molecule/buffer solution and vortexed to mix. The final concentration was 1.4 μM V599E Braf kinase and 20 μM small molecule. The final solution was transferred to a 50 μL cuvette for analysis.

Test solutions containing only the V599E Braf kinase were prepared as follows: 50 μL of 2.8 μM B-Raf protein was added to 50 μL Buffer and vortexed to mix, giving a concentration of 1.4 μM B-Raf in Buffer. The final solution was transferred to a 50 μL cuvette for analysis.

UV analysis was performed on an Agilent 8453 or 8452A U/V is spectrophotometer equipped with a Peltier temperature controller. The temperature profile increased from 25° C. to 70° C. in 0.2° C. increments over 8 hours with UV monitoring at 240 nm. A first derivative calculation with data smoothing was applied to the resulting data to report the point of inflection as the melting temperature. These data are exemplified in Table 26.

TABLE 26

| | ΔTm (° C.) relative |
|---|---|
| | to |
| | unphosphorylated |
| Example | B-Raf(V599E) |
| 38 | 4.7 |
| 66 | 15.8 |
| 79 | 12.0 |
| 80 | 14.6 |
| 81 | 17.3 |
| 82 | 13.5 |
| 83 | 14.9 |
| 84 | 12.5 |
| 85 | 2.9 |

### Thermal Denaturation of Abl Kinase

The binding of switch control inhibitors into Abl kinase was demonstrated using thermal denaturation by comparing the temperature at which the protein in solution with the small molecule becomes denatured (melts) compared to the melt temperature of the protein in solution alone (apo protein). Denaturation was monitored by measuring the absolute absorbance of the solutions at an appropriate wavelength (typically 230 nm and 240 nm) as a function of temperature. As the protein became denatured, additional UV absorbing functional groups became exposed causing an increase in absorbance at the melting temperature. Increased compound affinity was marked by a shift in the melting temperature to higher values. The thermal data were collected using Agilent spectrophotometers and ChemStation UV/Vis Thermal Denaturation software.

Test solutions containing both small molecule and Abl kinase were prepared as follows: 2 μL of a 10 mM small molecule in DMSO solution was mixed with 998 μL Buffer (Bis-T is Propane 19 mM pH7, NaCl 86.5 mM, EDTA 1.73 mM, 0.15% (w/v) octyl β-D-glucopyranoside (OG), 3.5% (v/v) DMSO) to give a concentration of 20 μM small molecule in buffer. 5 μL of 40 μM Abl kinase was mixed with 195 μL of the small molecule/buffer solution. The final concentration was 1 μM Abl kinase and 20 μM small molecule. The final protein and buffer solution was mixed gently using a pipetter to minimize denaturing due to over aggressive mixing. The final solution was transferred to a 50 μL cuvette for analysis.

Test solutions containing only the Abl kinase were prepared as follows: 5 μL of 40 μM Abl kinase was mixed with 195 μL Buffer to give a concentration of 1 μM Abl kinase in Buffer. The protein and buffer solution was mixed gently using a pipetter to minimize denaturing due to over aggressive mixing. The final solution was transferred to a 50 μL cuvette for analysis.

UV analysis was performed on an Agilent 8453 or 8452A UV/Vis spectrophotometer equipped with a Peltier temperature controller. The temperature profile increased from 25° C. to 70° C. in 0.2° C. increments over 8 hours with UV monitoring at 240 nm. A first derivative calculation with data smoothing was applied to the resulting data to report the point of inflection as the melting temperature. These data are exemplified in Table 27.

TABLE 27

| | ΔTm (° C.) relative |
|---|---|
| | to |
| | unphosphorylated |
| Example | Abl |
| 64 | 2.6 |
| 66 | 3.2 |
| 80 | 2.3 |
| 86 | 4.0 |
| 87 | 3.6 |
| 88 | 1.2 |
| 89 | 1.8 |
| 90 | 1.3 |
| 91 | 12.5 |

### Example D

### Step 6. Confirm Switch Control Mechanism of Protein Modulation

Small molecules that are found to have affinity for the protein or to exhibit functional modulation of protein activity are paced through biochemical studies to determine that binding or functional modulation is non-competitive or uncompetitive with natural ligand sites (e.g. The ATP site for kinase proteins). This is accomplished using standard biochemical analyses.

By way of example, small molecule candidate switch inhibitors were evaluated in a p38-alpha kinase biochemical assay and demonstrated to exhibit ATP-noncompetitive behavior.
Spectrophotometric Assay for Phospho-p38-alpha Kinase

### A. Materials

1. Phospho-p38-alpha from Roche Applied Diagnostics (0.8 mg/mL (12.5 uM))
2. p38 substrate: IPTSPITTTYFFFKKK-OH (>95% pure) from Biopeptide.
3. Pyruvate kinase (PK) and lactate dehydrogenase (LDH) from Sigma: 806 units of PK and 1100 units of LDH per mL
4. NADH (Sigma): 5.6 mM
5. Phosphoenol pyruvate (Sigma): 20 mM
6. Tris buffer: 100 mM Tris-HCl/20 mM MgCl₂, pH 7.5, 150 uM n-Dodecyl-b-D-Maltopyranoside, and 5% DMSO
7. ATP (20 mM)
8. 384 microplate (Corning 3675)
9. Polarstar Optima plate reader (BMG)

### B. Procedure for IC₅₀ Determination

IC₅₀ determinations were performed using a serial dilution scheme to dilute inhibitor in DMSO, followed by further dilution in reaction buffer, and finally mixing with Mixture 1 (Table 29) which contains all the enzymes. The dilution procedure ensured that the test compound at various concentrations was properly dissolved into the buffer and enzyme reaction mixture.

### C. Procedure:

1. Serially dilute test compound in 100% DMSO and in the reaction buffer according to Steps 1 and 2 of Table 28
2. Prepare Mixture 1 as described in Table 29 (use within 30 min)
3. Mix the diluted compound with Mixture 1 following Step 3 of Table 28
4. Incubate the 384-well plate at 30° C. for 2 hours
5. Add 1 μL of 30 mM ATP into each well and mix well
6. Read on the microplate reader with one point every min for at least 2.5 hours at 30° C.

TABLE 28
Scheme for IC₅₀ determination: Compound serial dilution and mixing
protocol

| | | | Well | | | | | | | | | |
|---|---|---|---|---|---|---|---|---|---|---|---|---|
| Step | | | 1 | 2 | 3 | 4 | 5 | 6 | 7 | 8 | 9 | 10 |
| 1 | Plate 1 (96 well) | | | | | | | | | | | |
| | Add 10 mM stock | | 2 uL* | | | | | | | | | |
| | Add 100% DMSO | in 100% | 198 uL | 1:1 serial dilution (100 uL at each well) | | | | | | | | |
| | Inhibitor, uM | DMSO | | 50 | 25 | 12.5 | 6.25 | 3.125 | 1.5625 | 0.78125 | 0.39063 | 0.19531 |
| | | | 100 | | | | | | | | | |
| 2 | Plate 2 (96 well) | | | | | | | | | | | |
| | Add the Tris buffer | | 245 uL | 245 uL | 245 uL | 245 uL | 245 uL | 245 uL | 245 uL | 245 uL | 245 uL | 245 uL |
| | with 5% DMSO** | | | | | | | | | | | |
| | Remove from plate 1 | | 5 uL | 5 uL | 5 uL | 5 uL | 5 uL | 5 uL | 5 uL | 5 uL | 5 uL | 5 uL |
| | Inhibitor, uM | in 7% | 2 | 1 | 0.5 | 0.25 | 0.125 | 0.0625 | 0.03125 | 0.01563 | 0.00781 | 0.00391 |
| | | DMSO | | | | | | | | | | |
| 3 | Plate 3 (384 well | | | | | | | | | | | |
| | Rxn plate) | | | | | | | | | | | |
| | Remove from plate | | 50 | 50 | 50 | 50 | 50 | 50 | 50 | 50 | 50 | 50 |
| | 2, uL | | | | | | | | | | | |
| | Add Mixture 1, uL | | 50 | 50 | 50 | 50 | 50 | 50 | 50 | 50 | 50 | 50 |
| | Final inhibitor, uM | in 3.5% | 1.0 | 0.50 | 0.25 | 0.125 | 0.0625 | 0.0313 | 0.0156 | 0.0078 | 0.0039 | 0.0020 |
| | | DMSO | | | | | | | | | | |

*The amount used in Step 1 is determined by the highest desired final compound concentration. In this procedure, the highest screening compound is 1 uM (see Step 3, “Final inhibitor, uM”). If the highest screening concentration is to be 100 nM, then 2 ul of 1 mM stock will be used in Step 1.
**Made by adding DMSO (final 5%) into the Tris buffer.

TABLE 29
Composition of Mixture 1

| | | Addition (uL) for | 10 mL of | Final concn in rxn | |
|---|---|---|---|---|---|
| | | 1 ml of Mixture | Mixture 1 (for | mixture | |
| Mixture 1 | Stocks | 1 (for 20 rxn) | 200 rxn) | (100 uL) | |
| PK/LDH | 806 u PK, 1100 u | 100 | 1000 | 40 u PK and 55 u | |
| | LDH per mL | | | LDH | |
| PEP | 20 mM | 100 | 1000 | 1 | mM |
| NADH | 4 mg/mL, 5.6 mM | 100 | 1000 | 280 | uM |
| Tris buffer (No | | 670 | 6700 | ~60 | mM |
| DMSO) | | | | | |
| P38 pep substrate | 10 mM | 40 | 400 | 200 | nM |
| P38a | 0.8 mg/mL, 12.5 uM | 0.7 | 7 | 4.4 | nM |
| | Total Volume, uL | 1011 | 10107 | | |

### D. Data Analysis for the Continuous Spectrophotometric Assay

A linear regression was applied to data collected from t=1.5 hr to 2.5 hr to obtain the reaction rate for each data set. The data were approximately linear within this time frame. Percentage of inhibition was calculated by comparison of the rate in the presence of a compound with that of the control. IC₅₀ values were calculated from a series of % inhibition values determined at a range of inhibitor concentrations using GraphPad Prism (version 4). The extinction coefficient at 340 nm for NADH under the assay condition (100 μL height in a Corning 3675 microplate well) was determined to be 5×10³ M⁻¹.

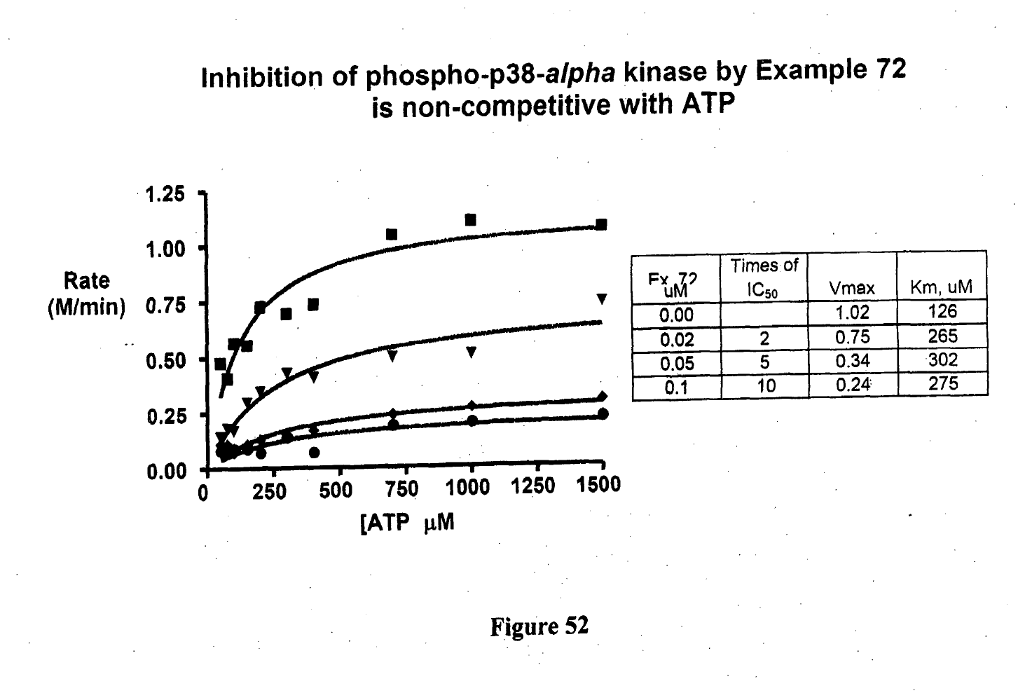

FIG. 52 illustrates that the small molecule of Example 72 is non-competitive with ATP.

### Use of X-Ray Crystallography to Determine Switch Control Inhibitor Binding Mechanism.

The mode of binding of switch control modulators to the various proteins are determined by X-ray crystallography or NMR techniques. The following section outlines the X-ray crystallography techniques used to determine the molecular mode of binding.

1. Crystallization Laboratory: All crystallization trial data were captured using a custom built database software which is used to drive a variety of robotic devices that set up crystallization trials and monitor the results. Computer Hardware that was used included Multiple Linux workstations, Windows 2000 servers, and Silicon Graphics O2 workstations. X-ray crystallography software included HKL2000, DENZO and SCALEPACK (X-ray diffraction data processing); MOSFILM; CCP4 suite, including AMORE, MOLREP and REFMAC (a variety of crystallographic computing operations, including phasing by molecular replacement, MIR, and MAD); SnB (for heavy atom location); SHARP (heavy atom phasing program); CNX (a variety of crystallographic computing operations, including model refinement); EPMR (molecular replacement); XtalView (model visualization and building).

2. Crystal Growth and X-ray Diffraction Quality Analysis: Sparse matrix and focused crystallization screens were set up with and without ligands at two or more temperatures. Crystals obtained without ligands (apo-crystals) were used for ligand soaking experiments. Once suitable protein-crystals had been obtained, a screen was performed to determine the diffraction quality of the protein-crystals under various cryo-preservation conditions on an R-AXIS IV imaging plate system and an X-STREAM cryostat. Protein-crystals of sufficient diffraction quality were used for X-ray diffraction data collection in-house, or stored in liquid nitrogen and saved for subsequent data collection at a synchrotron X-ray radiation source at the COM-CAT beamline at the Advanced Photon Source at Argonne National Laboratory or another synchrotron beam-line. The diffraction limits of protein-crystals were determined by taking at least two diffraction images at phi spindle settings 90° apart. The phi spindles were oscillated 1 degree during diffraction image collection. Both images were processed by the HKL-2000 suite of X-ray data analysis and reduction software. The diffraction resolution of the protein-crystals were accepted as the higher resolution limit of the resolution shell in which 50% or more of the indexed reflections have an intensity of I sigma or greater.

3. X-ray Diffraction Data Collection: A complete data set was defined as having at least 90% of all reflections in the highest resolution shell had been collected. The X-ray diffraction data were processed (reduced to unique reflections and intensities) using the HKL-2000 suite of X-ray diffraction data processing software.

4. Structure Determination: The structures of the protein-small molecule complexes were determined by molecular replacement (MR) using one or more protein search models available in the PDB. If necessary, the structure determination was facilitated by multiple isomorphous replacement (MIR) with heavy atoms and/or multi-wavelength anomalous diffraction (MAD) methods. MAD synchrotron data sets were collected for heavy atom soaked crystals if EXAFS scans of the crystals (after having been washed in mother liquor or cryoprotectant without heavy atom) revealed the appropriate heavy atom signal. Analysis of the heavy atom data sets for derivatization were completed using the CCP4 crystallographic suite of computational programs. Heavy atom sites were identified by (|F_{PH}|−|F_{P}|)² difference Patterson and the (|F₊|−|F₋|)² anomalous difference Patterson map.

X-ray Crystallographic Structural Analysis of Switch Control Inhibitors Bound to Composite Control Pockets

Binding of Example 8 Switch Control Inhibitor to Unphosphorylated p38-alpha Kinase.

The structure of Example 8 is shown below.

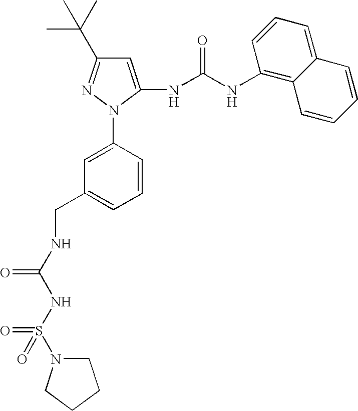

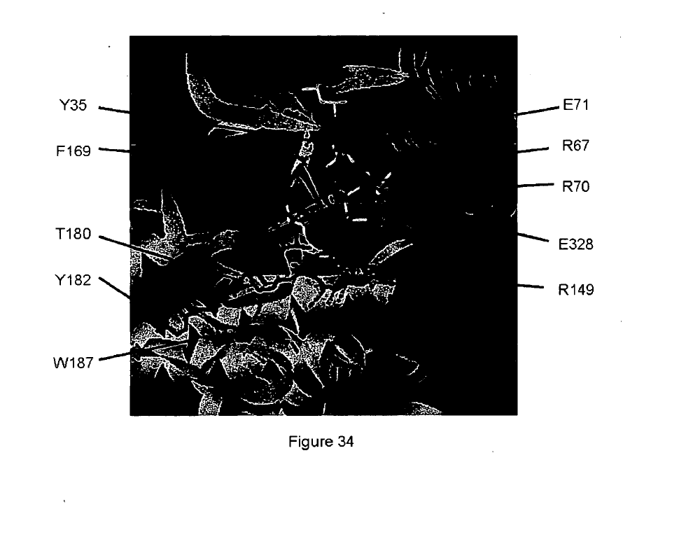

FIG. 34 illustrates the co-crystal structure of switch control inhibitor of Example 8 bound to p38-alpha kinase. The carbonyl moiety of the sulfonylurea of Example 8 makes a direct hydrogen-bond with the Z group arginine 70. Arginine 70 is a key anchoring group for stabilizing phosphorylated threonine 180 when the switch mechanism of p38-alpha kinase is in the on state. The opposite face of the guanidine side chain of arginine 70 makes direct hydrogen-bond and electrostatic interactions with glutamic acid 328, causing the dimerization domain (K-alpha helix amino acid residues isoleucine 334 through serine 347) to pack in against the C-alpha helix in a state unfavorable to dimeration or oligomerization of p38-alpha kinase. The sulfonyl moiety of the sulfonylurea of Example 8 makes an electrostatic interaction with histidine 174, a residue from the N-terminal region of the switch control ligand.

The urea moiety of Example 8 makes direct hydrogen-bond contact with the side chain of glutamic acid 71. Glutamic acid 71 is conserved in protein kinases, and serves a structural and catalytic role by engaging conserved lysine 53 from beta-strand 5.

The naphthyl moiety of Example 8 makes an edge-face π-stacking interaction with phenylalanine 169 (from the switch control ligand), stabilizing phenylalanine 169 in the out conformation and thereby occupying space in the ATP cofactor pocket. The naphthyl moiety also makes direct contact with the alkylene side chain of conserved lysine 53 of beta-strand 5, isoleucine 84 from beta-strand 6, and leucine 104 and threonine 106 from beta-strand 7.

The tertiary-butyl moiety of Example 8 occupies the ‘in conformation’ pocket that would otherwise be occupied by phenylalanine 169 if the switch mechanism of p38-alpha kinase were in the on state. The tertiary-butyl moiety makes hydrophobic contacts with leucine 74 and methionine 78 from the C-alpha helix; valine 83 from beta-strand 6; isoleucine 141 from the E-alpha helix; isoleucine 146 and histidine 148 from the catalytic loop.

In addition to these direct contacts between small molecule inhibitor Example 8 and the composite on switch pocket of p38-alpha kinase, other changes in the switch mechanism are induced by Example 8 to biomimetically down-regulate the biological activity of this protein kinase.

Specifically, the bulk of the switch control ligand from amino acid residues leucine 171 to glutamic acid 192 are placed into the binding pocket of the peptide or protein substrate, thereby blocking said substrate from binding to p38-alpha kinase.

Tyrosine 35 makes a π-cation electrostatic interaction with arginine 67 of the switch control pocket.

Aspartic acid 176, aspartic acid 177, and glutamic acid 178 form the off switch control pocket for binding and stabilizing unphosphorylated threonine 180. Tryptophan 187 forms an edge-face π-stacking interaction with unphosphorylated tyrosine 182.

Finally, glutamic acid 178 also forms a hydrogen-bond with switch control ligand residue threonine 185, orienting threonine 185 for interacting with catalytic amino acid residues aspartic acid 150, asparagine 155, and aspartic acid 168 by a network of direct and water-mediated hydrogen-bonds. This network of hydrogen-bonds induced by threonine 185 places these residues in a non-catalytic orientation.

Binding of Example 29 Switch Control Inhibitor to Unphosphorylated P38-alpha Kinase.

The structure of Example 29 is shown below.

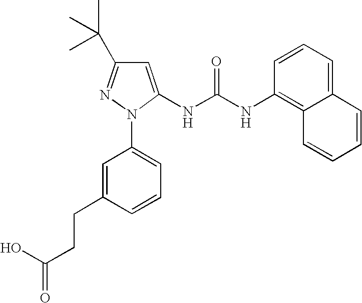

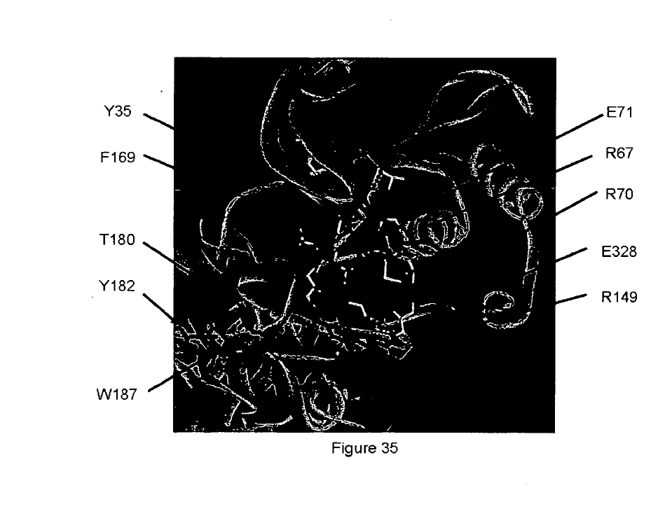

FIG. 35 illustrates the co-crystal structure of switch control inhibitor of Example 29 bound to p38-alpha kinase. The carboxylic acid moiety of Example 29 makes an electrostatic interaction with amino acid residues arginine 67 and tyrosine 35. Arginine 67 and tyrosine 35 biomimetically interact with each other when the switch mechanism of p38-alpha kinase is in the off state. The carboxylic acid side chain of Example 29 reinforces this interaction.

The urea moiety of Example 29 makes direct hydrogen-bond contact with the side chain of glutamic acid 71. Glutamic acid 71 is conserved in protein kinases, and serves a structural and catalytic role by engaging conserved lysine 53 from beta-strand 5.

The naphthyl moiety of Example 29 makes an edge-face π-stacking interaction with phenylalanine 169 (from the switch control ligand), stabilizing phenylalanine 169 in the out conformation and thereby occupying space in the ATP cofactor pocket. The naphthyl moiety also makes direct contact with the alkylene side chain of conserved lysine 53 of beta-strand 5, isoleucine 84 from beta-strand 6, and leucine 104 and threonine 106 from beta-strand 7.

The tertiary-butyl moiety of Example 29 occupies the ‘in conformation’ pocket that would otherwise be occupied by phenylalanine 169 if the switch mechanism of p38-alpha kinase were in the on state. The tertiary-butyl moiety makes hydrophobic contacts with leucine 74 and methionine 78 from the C-alpha helix; valine 83 from beta-strand 6; isoleucine 141 from the E-alpha helix; and histidine 148 from the catalytic loop.

In addition to these direct contacts between small molecule inhibitor Example 29 and the composite on switch pocket of p38-alpha kinase, other changes in the switch mechanism are induced by Example 29 to biomimetically down-regulate the biological activity of this protein kinase.

Specifically, the bulk of the switch control ligand from amino acid residues leucine 171 to glutamic acid 192 are placed into the binding pocket of the peptide or protein substrate, thereby blocking said substrate from binding to p38-alpha kinase.

The guanidine side chain of arginine 70 makes direct hydrogen-bond and electrostatic interactions with glutamic acid 328, causing the dimerization domain (K-alpha helix amino acid residues isoleucine 334 through serine 347) to pack in against the C-alpha helix in a state unfavorable to dimeration or oligomerization of p38-alpha kinase.

Tryptophan 187 forms an edge-face π-stacking interaction with unphosphorylated tyrosine 182.

There is no electron density for switch control ligand residues glycine 170 through glutamic acid 178. Nevertheless, the catalytic amino acid residues aspartic acid 150, asparagine 155, and aspartic acid 168 are placed in a non-catalytic orientation.

Binding of Example 61 Switch Control Inhibitor to Unphosphorylated p38-alpha Kinase.

The structure of Example 61 is shown below.

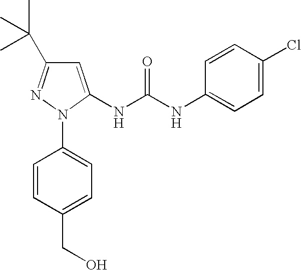

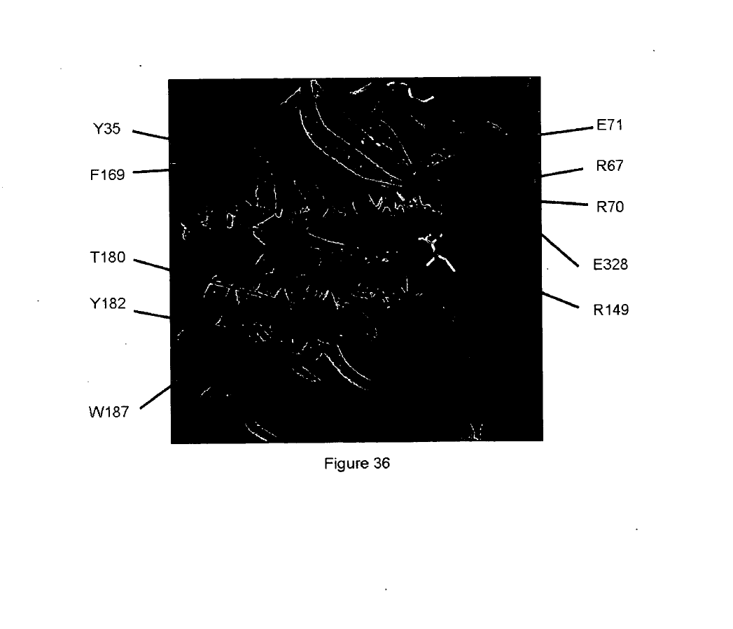

FIG. 36 illustrates the co-crystal structure of switch control inhibitor of Example 61 bound to p38-alpha kinase. The carbinol moiety of Example 61 makes a direct hydrogen-bond with the Z group arginine 70. Arginine 70 is a key anchoring group for stabilizing phosphorylated threonine 180 when the switch mechanism of p38-alpha kinase is in the on state. The opposite face of the guanidine side chain of arginine 70 makes direct hydrogen-bond and electrostatic interactions with glutamic acid 328, causing the dimerization domain (K-alpha helix amino acid residues isoleucine 334 through serine 347) to pack in against the C-alpha helix in a state unfavorable to dimeration or oligomerization of p38-alpha kinase.

The urea moiety of Example 61 makes direct hydrogen-bond contact with the side chain of glutamic acid 71. Glutamic acid 71 is conserved in protein kinases, and serves a structural and catalytic role by engaging conserved lysine 53 from beta-strand 5.

The para-chlorophenyl moiety of Example 61 makes an edge-face π-stacking interaction with phenylalanine 169 (from the switch control ligand), stabilizing phenylalanine 169 in the out conformation and thereby occupying space in the ATP cofactor pocket. The para-chlorophenyl moiety also makes direct contact with the alkylene side chain of conserved lysine 53 of beta-strand 5, isoleucine 84 from beta-strand 6, and leucine 104 and threonine 106 from beta-strand 7.

The tertiary-butyl moiety of Example 61 occupies the ‘in conformation’ pocket that would otherwise be occupied by phenylalanine 169 if the switch mechanism of p38-alpha kinase were in the on state. The tertiary-butyl moiety makes hydrophobic contacts with leucine 74 and methionine 78 from the C-alpha helix; valine 83 from beta-strand 6; isoleucine 141 from the E-alpha helix; and histidine 148 from the catalytic loop.

In addition to these direct contacts between small molecule inhibitor Example 61 and the composite on switch pocket of p38-alpha kinase, other changes in the switch mechanism are induced by Example 61 to biomimetically down-regulate the biological activity of this protein kinase.

Specifically, the bulk of the switch control ligand from amino acid residues leucine 171 to glutamic acid 192 are placed into the binding pocket of the peptide or protein substrate, thereby blocking said substrate from binding to p38-alpha kinase.

Tyrosine 35 makes a π-cation electrostatic interaction with arginine 67 of the switch control pocket.

Aspartic acid 176, aspartic acid 177, and glutamic acid 178 form the off switch control pocket for binding and stabilizing unphosphorylated threonine 180. Tryptophan 187 forms an edge-face π-stacking interaction with unphosphorylated tyrosine 182.

Finally, glutamic acid 178 also forms a hydrogen-bond with switch control ligand residue threonine 185, orienting threonine 185 for interacting with catalytic amino acid residues aspartic acid 150, asparagine 155, and aspartic acid 168 by a network of direct and water-mediated hydrogen-bonds. This network of hydrogen-bonds induced by threonine 185 places these residues in a non-catalytic orientation.

Binding of Example 62 Switch Control Inhibitor to Unphosphorylated p38-alpha Kinase.

The structure of Example 62 is shown below.

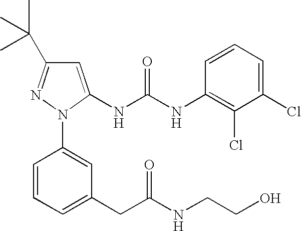

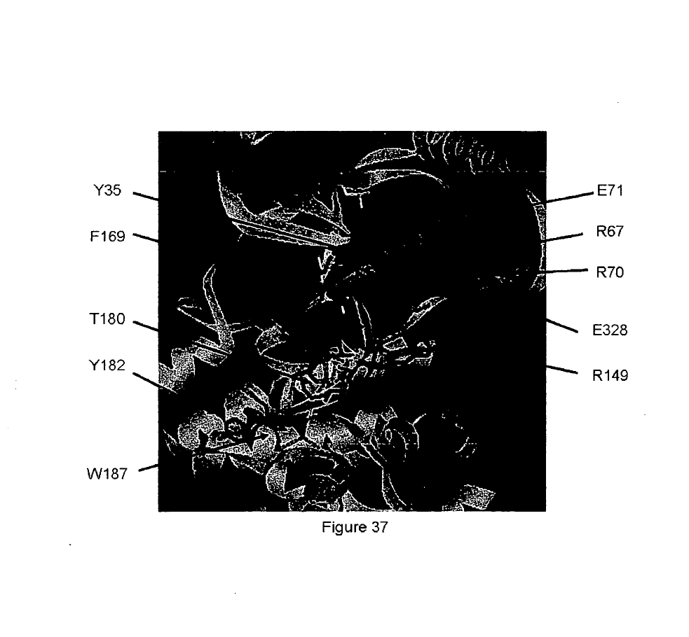

FIG. 37 illustrates the co-crystal structure of switch control inhibitor of Example 62 bound to p38-alpha kinase. The amide moiety of Example 62 makes a direct hydrogen-bond with the Z group arginine 70. Arginine 70 is a key anchoring group for stabilizing phosphorylated threonine180 when the switch mechanism of p38-alpha kinase is in the on state. The beta-hydroxy ethyl OH moiety of the amide binds into the switch pocket in a cis orientation together with the amide carbonyl moiety, acting to reinforce the hydrogen-bonding of the amide carbonyl with the guanidine side chain of arginine 70. The opposite face of the guanidine side chain of arginine 70 makes direct hydrogen-bond and electrostatic interactions with glutamic acid 328, causing the dimerization domain (K-alpha helix amino acid residues isoleucine 334 through serine 347) to pack in against the C-alpha helix in a state unfavorable to dimeration or oligomerization of p38-alpha kinase.

The urea moiety of Example 62 makes direct hydrogen-bond contact with the side chain of glutamic acid 71. Glutamic acid 71 is conserved in protein kinases, and serves a structural and catalytic role by engaging conserved lysine 53 from beta-strand 5.

The 2,3-dichlorophenyl moiety of Example 62 makes an edge-face π-stacking interaction with phenylalanine 169 (from the switch control ligand), stabilizing phenylalanine 169 in the out conformation and thereby occupying space in the ATP cofactor pocket. The 2,3-dichlorophenyl moiety also makes direct contact with the alkylene side chain of conserved lysine 53 of beta-strand 5, isoleucine 84 from beta-strand 6, and leucine 104 and threonine 106 from beta-strand 7. The meta-chloro substituent of the 2,3-dichlorophenyl moiety is within hydrogen-bonding distance of the main chain NH group of conserved amino acid residue lysine 53.

The tertiary-butyl moiety of Example 62 occupies the ‘in conformation’ pocket that would otherwise be occupied by phenylalanine 169 if the switch mechanism of p38-alpha kinase were in the on state. The tertiary-butyl moiety makes hydrophobic contacts with leucine 74 and methionine 78 from the C-alpha helix; valine 83 from beta-strand 6; isoleucine 141 from the E-alpha helix; and histidine 148 from the catalytic loop.

In addition to these direct contacts between small molecule inhibitor Example 62 and the composite on switch pocket of p38-alpha kinase, other changes in the switch mechanism are induced by Example 62 to biomimetically down-regulate the biological activity of this protein kinase.

Specifically, the bulk of the switch control ligand from amino acid residues leucine 171 to glutamic acid 192 are placed into the binding pocket of the peptide or protein substrate, thereby blocking said substrate from binding to p38-alpha kinase.

Tyrosine 35 makes a π-cation electrostatic interaction with arginine 67 of the switch control pocket.

Aspartic acid 176, aspartic acid 177, and glutamic acid 178 form the off switch control pocket for binding and stabilizing unphosphorylated threonine 180. Tryptophan 187 forms an edge-face π-stacking interaction with unphosphorylated tyrosine 182.

Finally, glutamic acid 178 also forms a hydrogen-bond with switch control ligand residue threonine 185, orienting threonine 185 for interacting with catalytic amino acid residues aspartic acid 150, asparagine 155, and aspartic acid 168 by a network of direct and water-mediated hydrogen-bonds. This network of hydrogen-bonds induced by threonine 185 places these residues in a non-catalytic orientation.

Binding of Example 63 Switch Control Inhibitor to Unphosphorylated p38-alpha Kinase.

The structure of Example 63 is shown below.

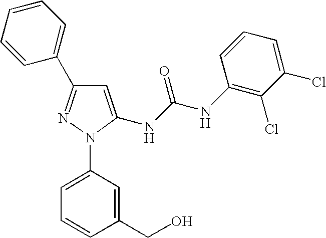

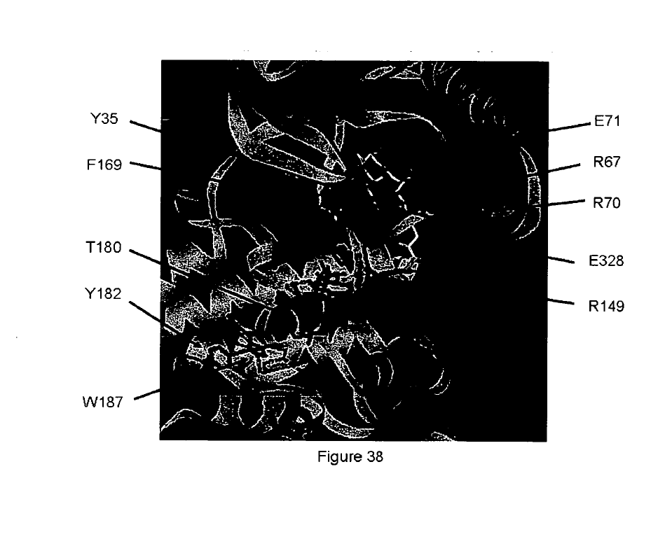

FIG. 38 illustrates the co-crystal structure of switch control inhibitor of Example 63 bound to p38-alpha kinase. The carbinol moiety of Example 63 makes a direct hydrogen-bond with the Z group arginine 70. Arginine 70 is a key anchoring group for stabilizing phosphorylated threonine 180 when the switch mechanism of p38-alpha kinase is in the on state. The opposite face of the guanidine side chain of arginine 70 makes direct hydrogen-bond and electrostatic interactions with glutamic acid 328, causing the dimerization domain (K-alpha helix amino acid residues isoleucine 334 through serine 347) to pack in against the C-alpha helix in a state unfavorable to dimeration or oligomerization of p38-alpha kinase.

The urea moiety of Example 63 makes direct hydrogen-bond contact with the side chain of glutamic acid 71. Glutamic acid 71 is conserved in protein kinases, and serves a structural and catalytic role by engaging conserved lysine 53 from beta-strand 5.

The 2,3-dichlorophenyl moiety of Example 63 makes an edge-face π-stacking interaction with phenylalanine 169 (from the switch control ligand), stabilizing phenylalanine 169 in the out conformation and thereby occupying space in the ATP cofactor pocket. The 2,3-dichlorophenyl moiety also makes direct contact with the alkylene side chain of conserved lysine 53 of beta-strand 5, isoleucine 84 from beta-strand 6, and leucine 104 and threonine 106 from beta-strand 7. The meta-chloro substituent of the 2,3-dichlorophenyl moiety is within hydrogen-bonding distance of the main chain NH group of conserved amino acid residue lysine 53.

The phenyl moiety (attached at the 3-position of the pyrazole ring) of Example 63 occupies the ‘in conformation’ pocket that would otherwise be occupied by phenylalanine 169 if the switch mechanism of p38-alpha kinase were in the on state. Phenyl moieties at this position on the pyrazole ring impart a high degree of selectivity toward p38-alpha kinase versus other kinases which do not tolerate phenyl or heteroaryl moieties at this position. The phenyl moiety makes hydrophobic contacts with leucine 74 and methionine 78 from the C-alpha helix; valine 83 from beta-strand 6; isoleucine 141 from the E-alpha helix; and isoleucine 146 and histidine 148 from the catalytic loop.

In addition to these direct contacts between small molecule inhibitor Example 63 and the composite on switch pocket of p38-alpha kinase, other changes in the switch mechanism are induced by Example 63 to biomimetically down-regulate the biological activity of this protein kinase.

Specifically, the bulk of the switch control ligand from amino acid residues leucine 171 to glutamic acid 192 is placed into the binding pocket of the peptide or protein substrate, thereby blocking said substrate from binding to p38-alpha kinase.

Tyrosine 35 makes a π-cation electrostatic interaction with arginine 67 of the switch control pocket.

Aspartic acid 176, aspartic acid 177, and glutamic acid 178 form the off switch control pocket for binding and stabilizing unphosphorylated threonine 180. Tryptophan 187 forms an edge-face π-stacking interaction with unphosphorylated tyrosine 182.

Finally, glutamic acid 178 also forms a hydrogen-bond with switch control ligand residue threonine 185, orienting threonine 185 for interacting with catalytic amino acid residues aspartic acid 150, asparagine 155, and aspartic acid 168 by a network of direct and water-mediated hydrogen-bonds. This network of hydrogen-bonds induced by threonine 185 places these residues in a non-catalytic orientation.

Binding of Example 29 Switch Control Inhibitor to Doubly Phosphorylated P38-alpha Kinase.

The structure of Example 29 is shown below.

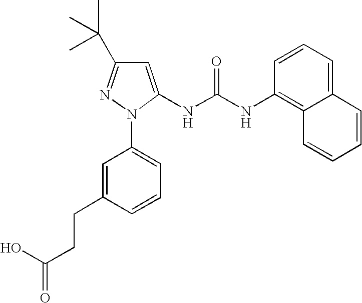

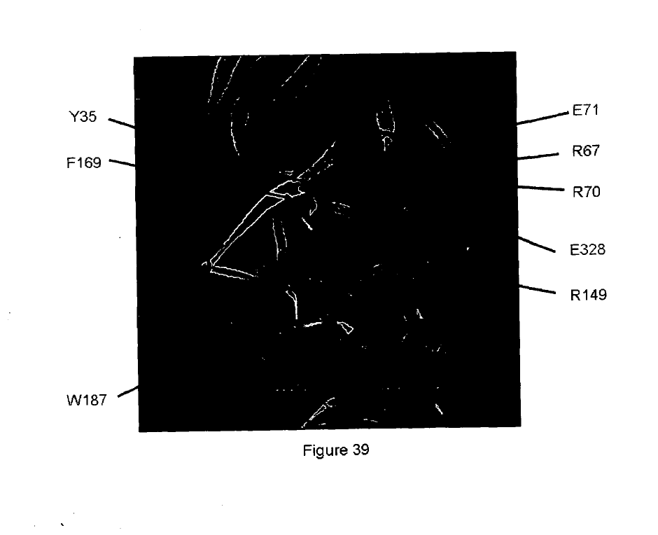

FIG. 39 illustrates the co-crystal structure of switch control inhibitor of Example 29 bound to doubly phosphorylated p38-alpha kinase. The carboxylic acid moiety of Example 29 makes an electrostatic interaction with amino acid residues arginine 67 and tyrosine 35. Arginine 67 and tyrosine 35 biomimetically interact with each other when the switch mechanism of p38-alpha kinase is in the off state. The carboxylic acid side chain of Example 29 reinforces this interaction.

The urea moiety of Example 29 makes direct hydrogen-bond contact with the side chain of glutamic acid 71. Glutamic acid 71 is conserved in protein kinases, and serves a structural and catalytic role by engaging conserved lysine 53 from beta-strand 5.

The naphthyl moiety of Example 29 makes an edge-face π-stacking interaction with phenylalanine 169 (from the switch control ligand), stabilizing phenylalanine 169 in the out conformation and thereby occupying space in the ATP cofactor pocket. The naphthyl moiety also makes direct contact with the alkylene side chain of conserved lysine 53 of beta-strand 5, isoleucine 84 from beta-strand 6, and leucine 104 and threonine 106 from beta-strand 7.

The tertiary-butyl moiety of Example 29 occupies the ‘in conformation’ pocket that would otherwise be occupied by phenylalanine 169 if the switch mechanism of p38-alpha kinase were in the on state. The tertiary-butyl moiety makes hydrophobic contacts with leucine 74 and methionine 78 from the C-alpha helix; valine 83 from beta-strand 6; isoleucine 141 from the E-alpha helix; and histidine 148 from the catalytic loop.

In addition to these direct contacts between small molecule inhibitor Example 29 and the composite on switch pocket of p38-alpha kinase, other changes in the switch mechanism are induced by Example 29 to biomimetically down-regulate the biological activity of this protein kinase. Despite the switch control ligand of p38-alpha kinase being doubly phosphorylated at phosphothreonine 180 and phosphotyrosine 182 and thereby preferring to be in the on switch state, the small molecule switch inhibitor Example 29 forces the doubly phosphorylated switch control ligand into the off switch state.

Specifically, the bulk of the switch control ligand from amino acid residues leucine 171 to glutamic acid 192 are placed into the binding pocket of the peptide or protein substrate, thereby blocking said substrate from binding to p38-alpha kinase.

The guanidine side chain of arginine 70 makes direct hydrogen-bond and electrostatic interactions with glutamic acid 328, causing the dimerization domain (K-alpha helix amino acid residues isoleucine 334 through serine 347) to pack in against the C-alpha helix in a state unfavorable to dimeration or oligomerization of p38-alpha kinase.

Tryptophan 187 is located at its biomimetic location for accepting an edge-face π-stacking interaction with unphosphorylated tyrosine 182. However, since tyrosine 182 is phosphorylated and altered so as not to recognize tryptophan 187, phosphotyrosine 182 is disordered and is displaced into solvent space. There is also no electron density observed for phosphothreonine 180. Unlike unphosphorylated threonine 180, which binds into the off switch control pocket moieties aspartic acid 176, aspartic acid 177, and glutamic acid 178, the phosphorylated threonine 180 does not bind into this off switch pocket. Indeed, negatively charged electrostatic repulsion between phosphorylated threonine 180, aspartic acid 176, aspartic acid 177, and glutamic acid 178 result in a displacement of aspartic acid 176, aspartic acid 177, and glutamic acid 178 into solvent space.

Threonine 185 is in its biomimetic off switch position, and forms a water-mediated hydrogen-bond network with histidine 148 and aspartic acid 150, orienting these catalytic amino acid residues into a catalytically incompetent off state.

### Binding of Example 64 Switch Control Inhibitor Abl Kinase.

The structure of Example 64 is shown below.

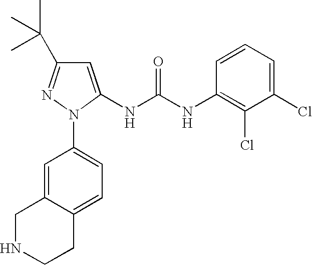

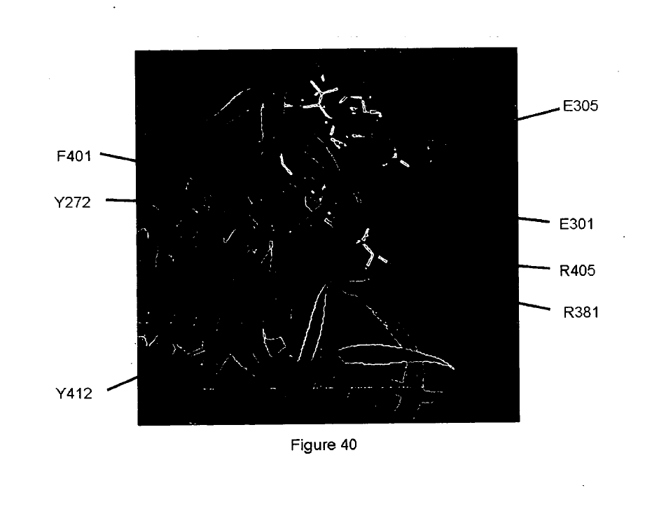

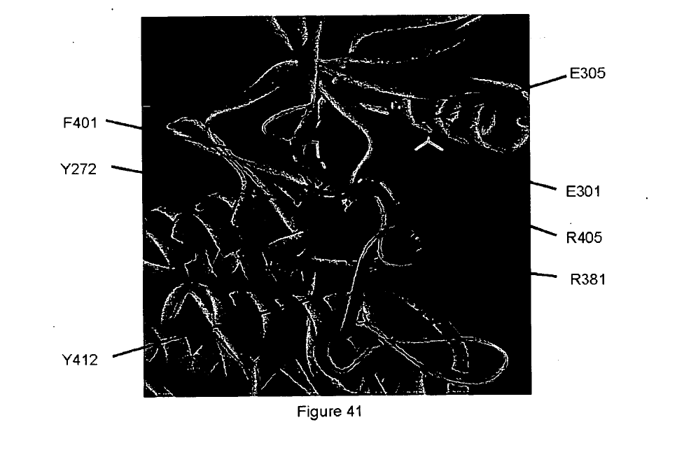

FIG. 40 illustrates the co-crystal structure of switch control inhibitor of Example 64 bound to Abl kinase. The ring amine functionality of the tetrahydroisoquinoline ring of Example 64 makes an electrostatic interaction with glutamic acid 301 from the C-alpha helix. Glutamic acid 301 acts as part of the switch mechanism of Abl kinase to stabilize the Z group arginine 405 in the off switch state. The ring amine functionality of the tetrahydroisoquinoline ring of Example 64 reinforces this type of electrostatic interaction with glutamic acid 301.

The urea moiety of Example 64 makes direct hydrogen-bond contact with the side chain of glutamic acid 305. Glutamic acid 305 is conserved in protein kinases, and serves a structural and catalytic role by engaging conserved lysine 290 from beta-strand 3.

The 2,3-dichlorophenyl moiety of Example 64 makes an edge-face π-stacking interaction with phenylalanine 401 (from the switch control ligand), stabilizing phenylalanine 401 in the out conformation and thereby occupying space in the ATP cofactor pocket. The 2,3-dichlorophenyl moiety also makes direct contact with the alkylene side chain of conserved lysine 290 of beta-strand 3, valine 318 from beta-strand 5, and isoleucine 332 and threonine 334 from beta-strand 6. The meta-chloro substituent of the 2,3-dichlorophenyl moiety is within hydrogen-bonding distance of the main chain NH group of conserved amino acid residue lysine 290.

The tertiary-butyl moiety of Example 64 occupies the ‘in conformation’ pocket that would otherwise be occupied by phenylalanine 401 if the switch mechanism of Abl kinase were in the on state. The tertiary-butyl moiety makes hydrophobic contacts with valine 308 from the C-alpha helix; isoleucine 312 from beta-strand 4; leucine 317 from beta-strand 5, leucine 373 from the E-alpha helix; and histidine 380 from the catalytic loop.

In addition to these direct contacts between small molecule inhibitor Example 64 and the composite on switch pocket of Abl kinase, other changes in the switch mechanism are induced by Example 64 to biomimetically down-regulate the biological activity of this protein kinase.

Specifically, the bulk of the switch control ligand from amino acid residues leucine 403 to isoleucine 422 is placed into the binding pocket of the peptide or protein substrate, thereby blocking said substrate from binding to Abl kinase.

Tyrosine 272 (from the glycine rich loop) is induced to form a π-stacking interaction with phenylalanine 401 on the face opposite to that of the 2,3-dichlorophenyl moiety's interaction with phenylalanine 401.

Switch control ligand amino acid residue tyrosine 412 is induced to bind into its biomimetic position, acting as a pseudosubstrate tyrosine binding into the catalytic amino acid residues aspartic acid 382 and asparagine 387 by a network of hydrogen-bonds. This network of hydrogen-bonds induced by tyrosine 412 places these residues in a non-catalytic orientation.

### Binding of Example 65 Switch Control Inhibitor to Abl Kinase.

The structure of Example 65 is shown below.

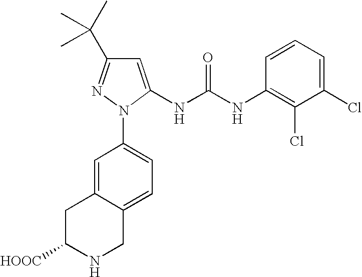

FIG. 41 illustrates the co-crystal structure of switch control inhibitor of Example 65 bound to Abl kinase. The amine and carboxylic acid functionalities of the tetrahydroisoquinoline ring of Example 65 makes an electrostatic interaction with glutamic acid 301 from the C-alpha helix and arginine 405 from the switch control ligand, respectively. Glutamic acid 301 acts as part of the switch mechanism of Abl kinase by stabilizing the Z group arginine 405 in the off switch state. The ring amine functionality of the tetrahydroisoquinoline ring of Example 65 also acts to reinforce this type of electrostatic interaction with glutamic acid 301, while the carboxylic acid functionality makes an electrostatic interaction with arginine 405.

The urea moiety of Example 65 makes direct hydrogen-bond contact with the side chain of glutamic acid 305. Glutamic acid 305 is conserved in protein kinases, and serves a structural and catalytic role by engaging conserved lysine 290 from beta-strand 3.

The 2,3-dichlorophenyl moiety of Example 65 makes a distorted edge-face n-stacking interaction with phenylalanine 401 (from the switch control ligand), stabilizing phenylalanine 401 in the out conformation and thereby occupying space in the ATP cofactor pocket. The 2,3-dichlorophenyl moiety also makes direct contact with the alkylene side chain of conserved lysine 290 of beta-strand 3, valine 318 from beta-strand 5, and isoleucine 332 and threonine 334 from beta-strand 6. The meta-chloro substituent of the 2,3-dichlorophenyl moiety is within hydrogen-bonding distance of the main chain NH group of conserved amino acid residue lysine 290.

The tertiary-butyl moiety of Example 65 occupies the ‘in conformation’ pocket that would otherwise be occupied by phenylalanine 401 if the switch mechanism of Abl kinase were in the on state. The tertiary-butyl moiety makes hydrophobic contacts with valine 308 from the C-alpha helix; isoleucine 312 from beta-strand 4; leucine 317 from beta-strand 5, leucine 373 from the E-alpha helix; and histidine 380 from the catalytic loop.

In addition to these direct contacts between small molecule inhibitor Example 65 and the composite on switch pocket of Abl kinase, other changes in the switch mechanism are induced by Example 64 to biomimetically down-regulate the biological activity of this protein kinase.

Specifically, the bulk of the switch control ligand from amino acid residues leucine 403 to isoleucine 422 is placed into the binding pocket of the peptide or protein substrate, thereby blocking said substrate from binding to Abl kinase.

Tyrosine 272 (from the glycine rich loop) is induced to form a π-stacking interaction with phenylalanine 401 on the face opposite to that of the 2,3-dichlorophenyl moiety's interaction with phenylalanine 401.

Switch control ligand amino acid residue tyrosine 412 is induced to bind into its biomimetic position, acting as a pseudosubstrate tyrosine binding into the catalytic amino acid residues aspartic acid 382 and asparagine 387 by a network of hydrogen-bonds. This network of hydrogen-bonds induced by tyrosine 412 places these residues in a non-catalytic orientation.

### Binding of Example 65 Switch Control Inhibitor to V599E Oncogenic Braf Kinase.

The structure of Example 65 is shown below.

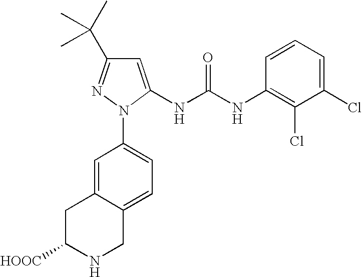

FIG. 42 illustrates the co-crystal structure of switch control inhibitor of Example 65 bound to oncogenic V599E Braf kinase. The carboxylic acid functionality of the tetrahydroisoquinoline ring of Example 65 makes a hydrogen bond interaction with the main chain carbonyl oxygen moiety of alanine 496 from the C-alpha helix. The carboxylic acid functionality of the tetrahydroisoquinoline ring also makes a hydrogen bond contact with asparagine 499. Asparagine 499 acts as either an on switch pocket Z group, stabilizing phosphothreonine 598, or alternatively acts as an off switch pocket X group, stabilizing arginine 602. The ring amine functionality of the tetrahydroisoquinoline ring of Example 65 was designed to potentially interact with the mutated oncogenic residue glutamic acid 599. However, no electron density is observed for glutamic acid 599.

The urea moiety of Example 65 makes direct hydrogen-bond contact with the side chain of glutamic acid 500. Glutamic acid 500 is conserved in protein kinases, and serves a structural and catalytic role by engaging conserved lysine 482 from beta-strand 3.

The 2,3-dichlorophenyl moiety of Example 65 makes an edge-face π-stacking interaction with phenylalanine 594 (from the switch control ligand), stabilizing phenylalanine 594 in the out conformation and thereby occupying space in the ATP cofactor pocket. The 2,3-dichlorophenyl moiety also makes direct contact with the alkylene side chain of conserved lysine 482 of beta-strand 3, leucine 513 from beta-strand 5, and isoleucine 526 and threonine 528 from beta-strand 6. The meta-chloro substituent of the 2,3-dichlorophenyl moiety is within hydrogen-bonding distance of the main chain NH group of conserved amino acid residue lysine 482.

The tertiary-butyl moiety of Example 65 occupies the ‘in conformation’ pocket that would otherwise be occupied by phenylalanine 594 if the switch mechanism of Braf kinase were in the on state. The tertiary-butyl moiety makes hydrophobic contacts with valine 503, leucine 504, and threonine 507 from the C-alpha helix; isoleucine 512 from beta-strand 5; leucine 566 from the E-alpha helix; and histidine 573 from the catalytic loop.

In addition to these direct contacts between small molecule inhibitor Example 65 and the composite on switch pocket of Braf kinase, other changes in the switch mechanism are induced by Example 65 to biomimetically down-regulate the biological activity of this protein kinase. Specifically, the bulk of the switch control ligand from amino acid residues leucine 596 to leucine 617 is placed into the binding pocket of the peptide or protein substrate, thereby blocking said substrate from binding to Braf kinase.

Switch control ligand amino acid residue threonine 598 is induced to bind into its biomimetic off switch position, binding into the catalytic amino acid residues aspartic acid 575, asparagine 580, and aspartic acid 593 by a network of water-mediated hydrogen-bonds. This network of hydrogen-bonds facilitated by threonine 598 places these residues in a non-catalytic orientation.

The binding of Example 65 into the on composite switch pocket of Braf kinase also induces the N-terminal switch control ligand hydrophobic amino acid residues phenylalanine 594, leucine 596, and alanine 597 to come into close contact with hydrophobic amino acid residues serine 466, phenylalanine 467, and valine 470 from the glycine rich loop (beta-strands 1 and 2). These hydrophobic interactions facilitate movement of the switch control ligand into its off switch state. Example 65 induces this biomimetic movement of the switch control ligand into its off state despite the mutation of a hydrophobic amino acid valine 599 to an activating glutamic acid 599.

### Example E

### Step 7. Iterate Above Steps to Improve Small Molecule Switch Control Modulators

Individual small molecules found to modulate protein activity were evaluated for affinity and functional modulation of other proteins within the protein superfamily (e.g., other kinases if the candidate protein is a kinase) or between protein families (e.g., other protein classes such as phosphatases and transcription factors if the candidate protein is a kinase). Small molecule screening libraries were also evaluated in this screening paradigm. Structure activity relationships (SARs) were assessed and small molecules were subsequently designed to be more potent for the candidate protein and/or more selective for modulating the candidate protein, thereby minimizing interactions with counter target proteins.

By way of illustration, an initial round of screening and experimentation identified the small molecule of Example 29 as a switch control inhibitor of p38-alpha kinase. Its properties in the various screening assays are summarized below:

### Example 29

Fluorescence affinity assay IC₅₀=16 nM (unphosphorylated p38-alpha kinase)

Fluorescence affinity assay IC₅₀=56 nM (phosphorylated p38-alpha kinase)

Thermal denaturation assay ΔTm=10.1° (unphosphorylated p38-alpha kinase)

Thermal denaturation assay ΔTm=7.3° (phosphorylated p38-alpha kinase)

Biochemical assay IC₅₀=12 nM

Interpretation of binding modes from X-ray co-crystal structures led to interative design and synthesis of small molecules exhibiting improved properties as switch control inhibitors of p38-alpha kinase. One such improved analog is Example 69, whose improved properties in the various screening assays are summarized below:

### Example 69

Fluorescence affinity assay IC₅₀=9 nM (unphosphorylated p38-alpha kinase)

Fluorescence affinity assay IC₅₀=11 nM (phosphorylated p38-alpha kinase)

Thermal denaturation assay ΔTm=13.10 (unphosphorylated p38-alpha kinase)

Thermal denaturation assay ΔTm=9.1° (phosphorylated p38-alpha kinase)

Biochemical assay IC₅₀=7.0 nM The analysis of the kinase proteins revealed four types of switch control pockets classified by their mode of binding to complemental switch control ligands, namely: (1) pockets which stabilize and bind to modified ligands, typically formed by phosphorylation of serine, threonine, or tyrosine amino acid residues in the complemental switch control ligands (charged ligand), or by oxidation of the sulfur atoms of methionine or cysteine amino acids; (2) pockets which bind to ligands through the mechanism of hydrogen bonding or hydrophobic interactions (H-bond/hydrophobic ligand); (3) pockets which bind ligands having acylated residues (acylated ligand); and (4) pockets which do not endogenously bind with a ligand, but which can bind with a non-naturally occurring switch control modulator compound (non-identified ligand). Further, these four types of pockets may be of the simple type schematically depicted in FIGS. 1-4, the composite type shown in FIGS. 6 and 6A, or the combined type of FIG. 7. Finally, the pockets may be defined by their switch control functionality, i.e., the pockets may be of the on variety which induces a biologically upregulated protein conformation upon switch control ligand interaction, the off variety which induces a biologically downregulated conformation upon switch control ligand interaction, or what is termed “dual functionality” pockets, meaning that the same pocket serves as both an on-pocket and an off-pocket upon interaction with different complemental switch control ligands (e.g. as exemplified with Gsk-3 beta kinase). This same spectrum of pockets can be found in all proteins of interest, i.e., those proteins which experience conformational changes via interaction of switch control ligand sequences and complemental switch control pockets.

The following Table 30 further identifies the pockets described in Steps 2 and 3 in terms of pocket classification and type.

TABLE 30

| | Identifying | |
|---|---|---|
| Protein | Table | Switch Control Pocket Type |
| Abl kinase | 5 | Charged ligand; Simple; -On |
| Abl kinase | 6 | Acylated ligand; Simple; -Off |
| p38-alpha kinase | 7 | Charged ligand; Simple; -On |
| Braf kinase | 8 | Charged ligand; Simple; -On |
| nic V599E Braf kinase | 8 | Charged ligand; Simple; -On |
| Gsk-3 beta kinase | 9 | Charged ligand; Simple; -Dual |
| Insulin receptor | 10 | Charged ligand; Simple; -On |
| kinase-1 | | |
| Protein kinase B/Akt | 11 | Charged ligand; Simple; -On |
| Transforming Growth | 12 | H-bond/hydrophobic; Simple; -Off |
| Factor B-I receptor | | |
| kinase | | |
| Transforming Growth | 13 | Non-identified ligand |
| Factor B-I receptor | | |
| kinase | | |
| Transforming Growth | 14 | Non-identified ligand |
| Factor B-I receptor | | |
| kinase | | |
| Abl kinase | 15 | Charged ligand; Composite; -On |
| Abl kinase | 16 | Charged ligand; Combined; -On |
| p38-alpha kinase | 17 | Charged ligand; Composite; -On |
| p38-alpha kinase | 18 | Charged ligand; Combined; -On |
| Braf kinase | 19 | Charged ligand; Composite; -On |
| Oncogenic V599E | 20 | Charged ligand; Composite; -On |
| Braf kinase | | |
| Gsk-3 beta kinase | 21 | Charged ligand; Composite; -Dual |

A principal aim of the invention is to facilitate the design and development of non-naturally occurring small molecule modulator compounds which will bind with selected proteins at the region of one or more of the switch control pockets thereof in order to modulate the activity of the protein. This functional goal can be achieved in several different ways, depending upon the type of switch control pocket (-on, -off, or -dual), the nature of the selected modulator compound, and the type of interactive binding between the modulator compound and the protein.

For example, a selected modulator compound may bind at the region of a selected switch control pocket as a switch control ligand agonist, i.e., the modulator compound effects the same type of conformational change as that induced by the naturally occurring, complemental switch control ligand. Thus, if a switch control ligand agonist binds with an on-pocket, the result will be up regulation of the protein activity, and if it binds with an off-pocket, down regulation occurs. Conversely, a given modulator may bind as a switch control ligand antagonist, i.e., the modulator compound effects the opposite type of conformational change as that induced by the naturally occurring, complemental switch control ligand. Hence, if a switch control ligand antagonist binds with an on-pocket, the result will be down regulation of the protein activity, and if it binds with an off-pocket, up regulation occurs.

In the case of dual functionality and non-identified liganded pockets, a modulator compound serves as a functional agonist or functional antagonist, depending upon on the type of response obtained.

Another aspect of the invention includes small molecule switch control inhibitors which bind simultaneously with some amino acid residues taken from an on composite switch control pocket and other amino acid residues taken from an off composite switch control pocket, of course including some Z or X residues as previously defined. Such inhibitors are categorized as dual on switch control pocket antagonists/off switch control pocket agonists. For example, in FIG. 35, the carboxylic acid-containing side chain of the small molecule of Example 29 stabilizes the interaction of arginine 67 (a conformational control Z residue from the on pocket) with tyrosine 35 (a conformational control X residue from the off pocket) in the off pocket. Additional small molecule/composite switch control pocket interactions exemplify the dual on switch control pocket antagonism/off switch control pocket agonism of Example 29: the naphthyl ring of the inhibitor stabilizes phenylalanine 169 in its off pocket conformational state, i.e., off switch control pocket agonism). Conversely, the tertiary-butyl moiety of the inhibitor occupies the binding site for phenylalanine 169, thereby precluding phenylalanine 169 from occupying its on pocket conformational state (i.e., on switch control pocket antagonism).

Another aspect of the invention includes small molecule switch control inhibitors which bind directly with a modifiable amino acid from the switch control ligand sequence. In this aspect of the invention, small molecule switch control inhibitors bind directly with serine, threonine, tyrosine, cysteine, or methionine residues in either their unmodified or modified states, or small molecule switch control inhibitors bind directly with amino acids of the switch control ligand sequence which are functionalized with fatty acid modifiers (e.g. myristoylated residues).

Yet another aspect of the invention includes small molecule switch control inhibitors which bind directly with mutant amino acids of a switch control ligand sequence which function to constituitively activate the switch mechanism. Such mutant amino acids include aspartic acids or glutamic acids. An example of such a mutant switch control ligand sequence is found in oncogenic V599E Braf kinase, wherein the wild type Braf contains a valine residue at position 599 in the switch control ligand sequence, whereas the oncogenic Braf contains a glutamic acid residue at position 599 which constituitively activates the kinase switch mechanism.

### Switch Control Pockets of Proteases and Modulation of These Pockets.

Caspase-3, a cysteine protease, has been found to be S-nitrosylated on cysteine residues in whole cells (J. B. Mannick et al, Fas-induced caspase denitrosylation, Science (1999) 284: 651; J. B. Mannick et al, Nitric oxide inhibits Fas-induced apoptosis, J. Biol. Chem. (1997) 272: 24125). Procaspase-3 (J. B. Mannick et al, Fas-induced caspase denitrosylation, Science (1999) 284: 651; J. B. Mannick et al, Nitric oxide inhibits Fas-induced apoptosis, J. Biol. Chem. (1997) 272: 24125) and procaspase-9 (J. E. Kim and S. R. Tannenbaum, S-Nitrosylation regulates the activation of endogenous procaspase-9 in HT-29 human carcinoma cells, J. Biol. Chem. (2004) 279: 9758) are nitrosylated in vivo on their respective catalytic cysteine residues. Moreover, activated caspases (wherein the zymogen procaspase has already been converted to the activated caspases) are also S-nitrosylated on their respective catalytic cysteine residues upon treatment with nitric oxide and the catalytic activity of those caspases is inhibited by S-nitrosylation (B. Zech et al, Mass spectrometric analysis of nitric oxide-modified caspase-3, J. Biol. Chem. (1999) 274: 20931; S. Mohr et al, Inhibition of caspase-3 by S-nitrosylation and oxidation caused by nitric oxide, Biochem. Biophys. Res. Commun. (1997) 238: 387; J. Li et al, Nitric oxide reversibly inhibits seven members of the caspase family via S-nitrosylation, Biochem. Biophys. Res. Commun. (1997) 240: 419). A different allosteric cysteine residue has been studied in caspase-3 and caspase-7 which when modified leads to inhibition of caspase catalytic activity (J. A. Hardy et al, Discovery of an allosteric site in the caspases, Proc. Natl. Acad USA (2004) 101: 12461). It is this second type of cysteine residue, the allosteric cysteine, which conforms to the definition of a switch control ligand in the present invention. Transient oxidation (S-nitrosylation) of these switch control ligand cysteine residues control the shape and hence biological activities of caspases. The present invention relates to the identification of switch control inhibitors of cysteine proteases, including caspases, that bind into the switch control pocket responsive to the transiently oxidized switch control ligand containing the S-nitrosylated cysteine.

The switch control ligand amino acid sequence and the amino acids comprising the switch control pocket of the cysteine protease caspase-7 having SEQ ID. NO. 57 are illustrated in the Table 31.

TABLE 31
Caspase-7 Switch Control Ligand (Chain A)

| | | | | | |
|---|---|---|---|---|---|
| B-strand 7 | | | | | |
| F219 | L220 | F221 | A222 | Y223 | |
| Loop 3 | | | | | |
| S224 | T225 | V226 | P227 | G228 | Y229 |
| Loop 4 | | | | | |
| I288 | P289 | | | | |
| B-strand 8 | | | | | |
| C290 | V291 | V292 | S293 | M294 | |

Caspase-7 Switch Control Pocket (Chain B)

| | | | | | |
|---|---|---|---|---|---|
| Loop 2 prime | | | | | |
| P209 | R210 | Y211 | K212 | I213 | P214 |
| Y215 | E216 | A217 | D218 | | |
| B-strand 7 prime | | | | | |
| F219 | L220 | F221 | A222 | Y223 | |
| B-strand 8 prime | | | | | |
| C290 | V291 | V292 | S293 | M294 | |

Cysteine residue 290 from chain A constitutes the modifiable residue that is transiently oxidized to an S-nitrosylated state. Arginine 210-prime from chain B is a key X group from the switch control pocket which stabilizes the modified switch control ligand in the off state.

### Synthesis of Potential Switch Control Small Molecules

The small molecule switch control inhibitors described herein were prepared by methods described in application Ser. No. 10/746,545, filed Dec. 24, 2003; Anti-inflammatory Medicaments, application Ser. No. 10/746,460, filed Dec. 24, 2003; Anti-cancer Medicaments, application Ser. No. 10/746,607, filed Dec. 24, 2003; the provisional applications entitled Process For Modulating Protein Function, Ser. No. 60/437,487 filed Dec. 31, 2002; Anti-Cancer Medicaments, Ser. No. 60/437,403 filed Dec. 31, 2002; Anti-Inflammatory Medicaments, Ser. No. 60/437,415 filed Dec. 31, 2002; Anti-Inflammatory Medicaments, Ser. No. 60/437,304 filed Dec. 31, 2002; U.S. Patent Application No. 60/638,987, filed Dec. 23, 2004, Enzyme Modulators For Treatment Of Inflammatory, Autoimmune, Cardiovascular And Immunological Diseases; U.S. Patent Application No. 60/639,087, filed Dec. 23, 2004, Enzyme Modulators For Treatment Of Cancers And Hyperproliferative Diseases; U.S. Patent Application No. 60/638,986, filed Dec. 23, 2004, Enzyme Modulators For Treatment Of Cancers, Hyperproliferative Disorders, Or Diseases Treatable With An Anti-Angiogenic Agent; and U.S. Patent Application No. 60/638,968, filed Dec. 23, 2004, Enzyme Modulators For Treatment Of Cancers And Hyperproliferative Diseases. Each of these applications is incorporated by reference herein.

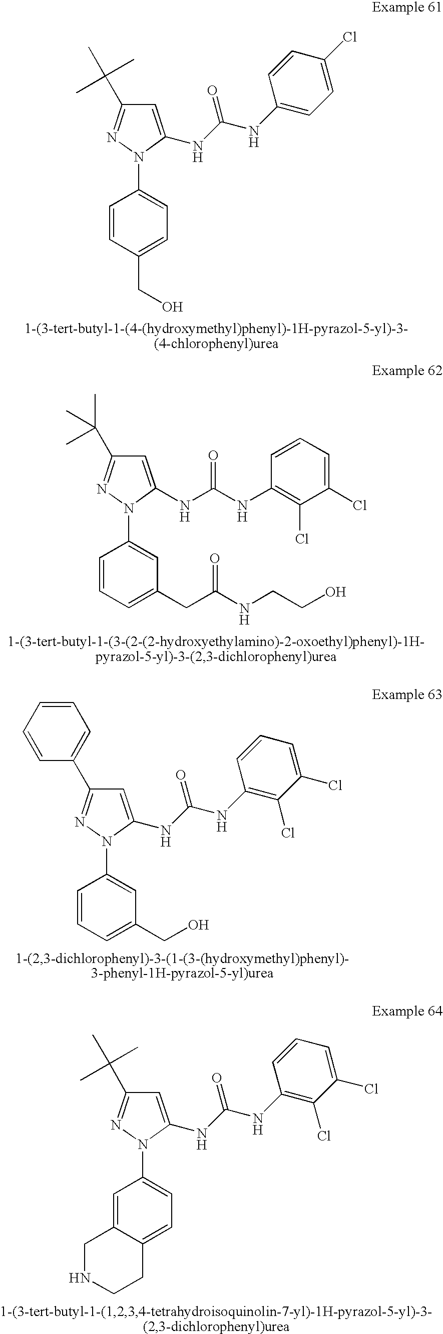

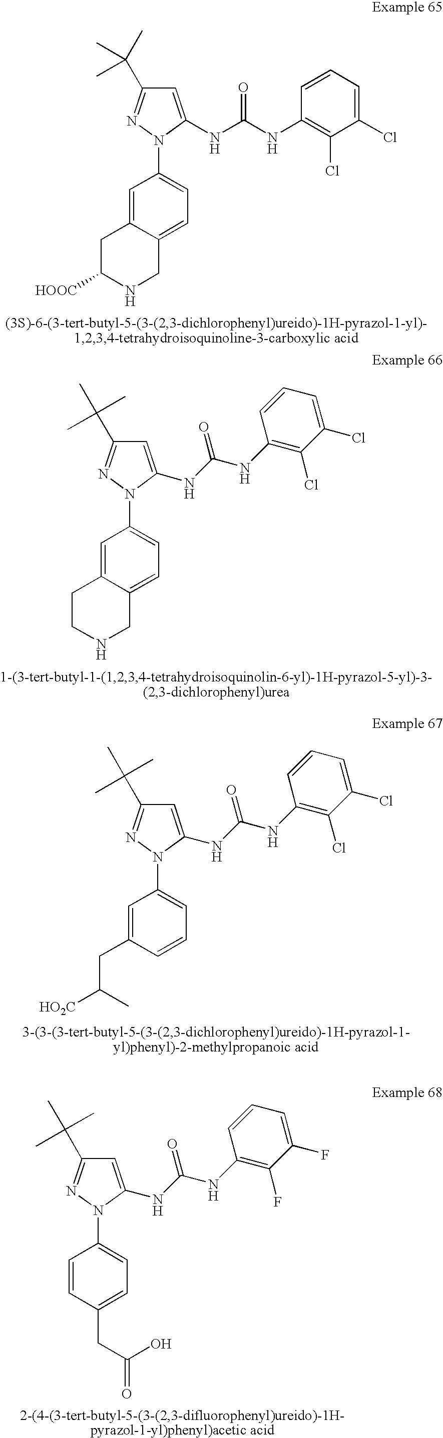

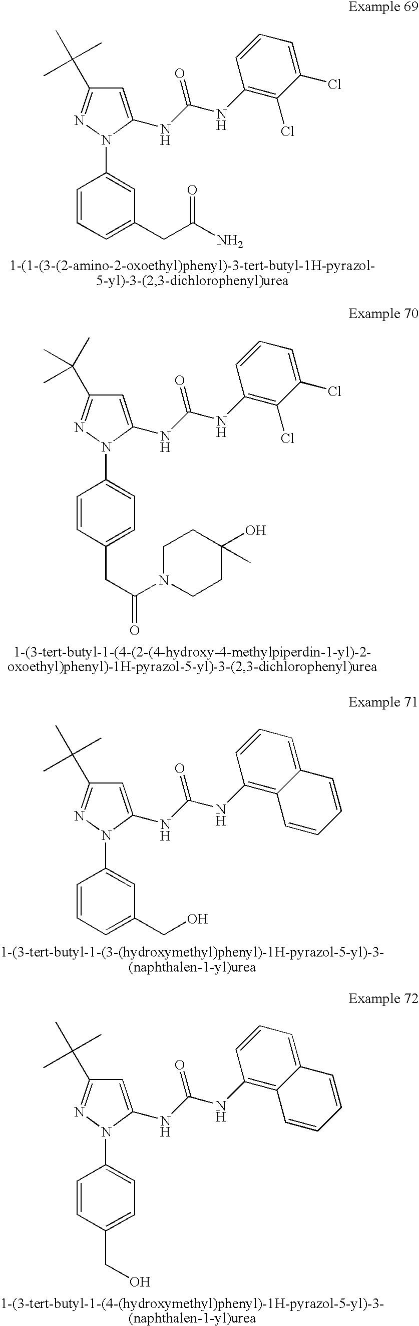

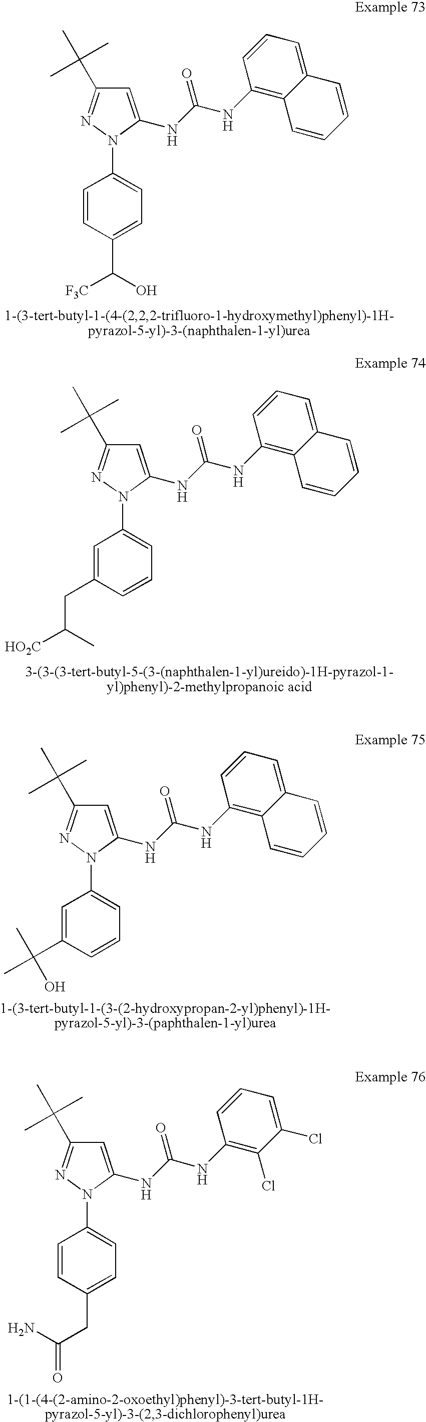

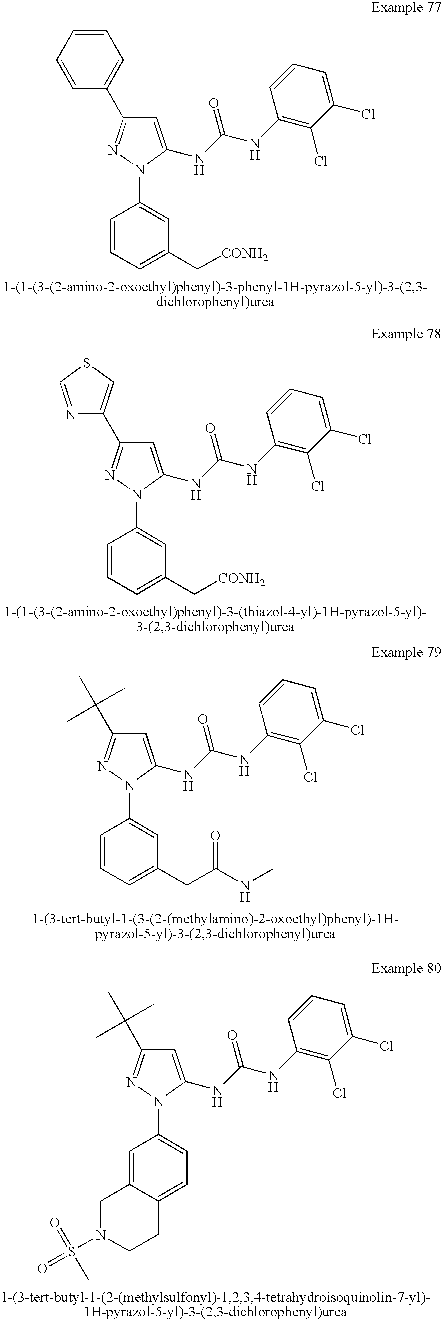

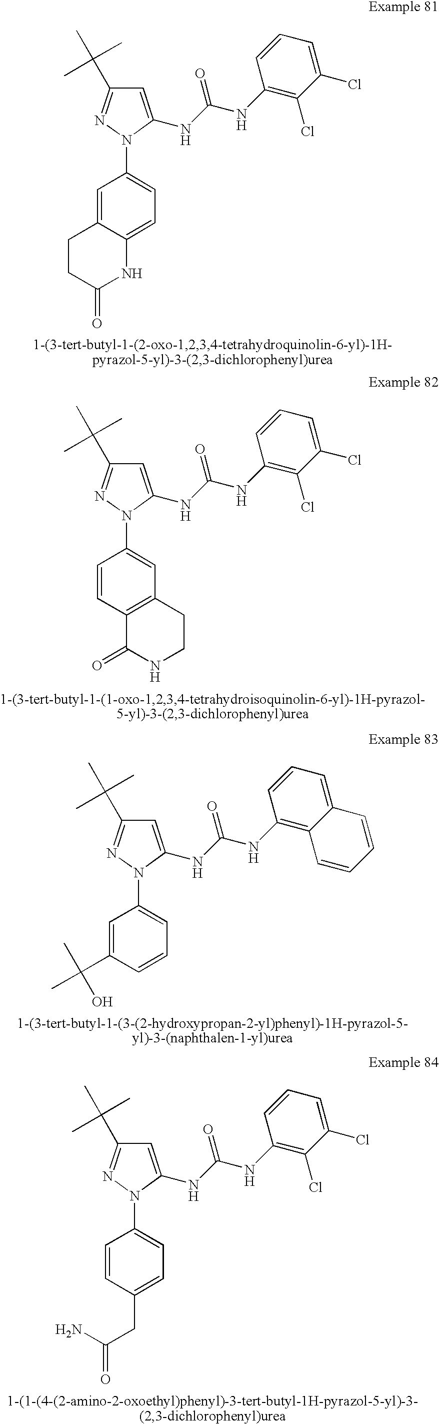

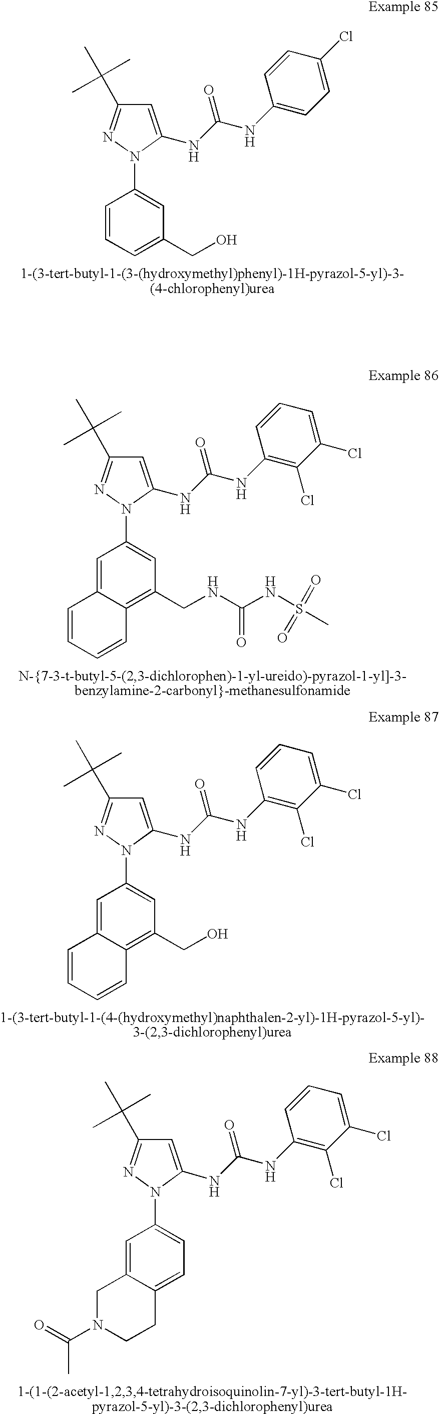

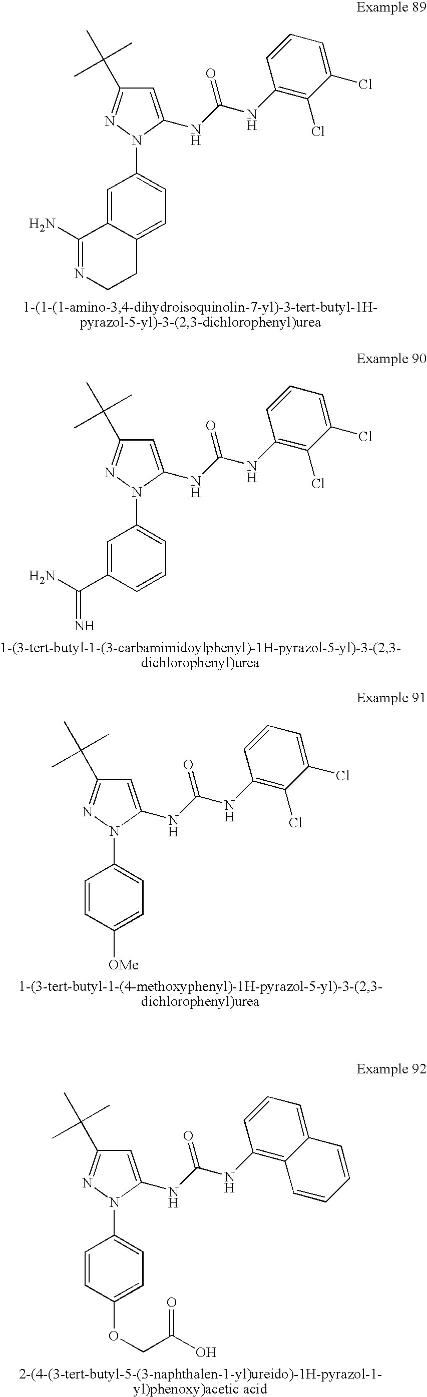

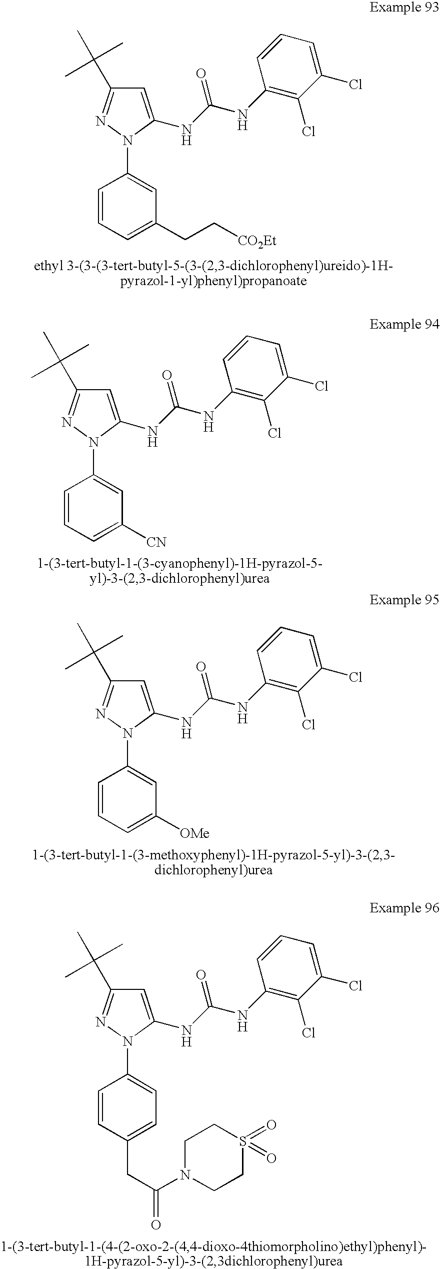

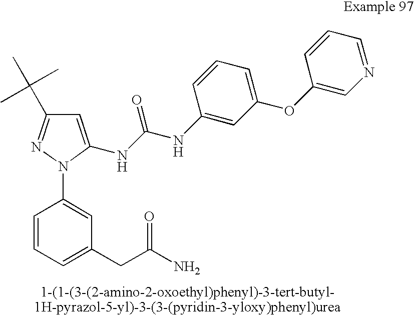

All of the references above identified are incorporated by reference herein. In addition, the following simultaneously applications are also incorporated by reference, namely AntiCancer Medicaments, Ser. No. 60/437,403 filed Dec. 31, 2002; Anti-Inflammatory Medicaments, Ser. No. 60/437,415 filed Dec. 31, 2002; Anti-Inflammatory Medicaments, Ser. No. 60/437,304 filed Dec. 31, 2002.

We claim:

1. A protein-modulator adduct comprising a naturally occurring protein having a switch control pocket and a switch control ligand, with a non-naturally occurring molecule bound to the protein at the region of said switch control pocket, said molecule serving to at least partially regulate the biological activity of said protein by inducing or restricting the conformation of the protein, said switch control pocket having a plurality of conformational control amino acid residues which are capable of binding with corresponding modifiable residues forming a part of said switch control ligand, said molecule being bound to at least one amino acid residue taken from the switch pocket conformational control amino acid residues or the switch control ligand modifiable amino acid residues.

2. The adduct of claim 1, said molecule serving to induce a conformation change in said protein.

3. The adduct of claim 1, said molecule serving to restrict a conformation change in said protein.

4. The adduct of claim 1, said ligand interacting in vivo with said pocket to regulate the conformation and biological activity of said protein such that the protein will assume a first conformation and a first biological activity upon said ligand-pocket interaction, and will assume a second, different conformation and biological activity in the absence of said ligand-pocket interaction.

5. The adduct of claim 1, said pocket being an on-pocket, said molecule binding with said protein at the region of said on-pocket as an agonist.

6. The adduct of claim 1, said pocket being an on-pocket, said molecule binding with said protein at the region of said on-pocket as an antagonist.

7. The adduct of claim 1, said pocket being an off-pocket, said molecule binding with said protein at the region of said off-pocket as an agonist.

8. The adduct of claim 1, said pocket being an off-pocket, said molecule binding with said protein at the region of said off-pocket as an antagonist.

9. The adduct of claim 1, said protein selected from the group consisting of enzymes, receptors, and signaling proteins.

10. The adduct of claim 9, said protein selected from the group consisting of kinases, phosphatases, phosphodiesterases, proteases, sulfotranferases, sulfatases, transcription factors, nuclear hormone receptors, g-protein coupled receptors, g-proteins, gtp-ases, hormones, polymerases, and other proteins containing nucleotide regulatory sites.

11. The adduct of claim 1, said protein having a molecular weight of at least about 15 kDa.

12. The adduct of claim 11, said molecular weight being above about 30 kDa.

13. The adduct of claim 1, said protein being a kinase protein.

14. A protein-modulator adduct comprising a naturally occurring protein with a non-naturally occurring molecule bound to the protein and serving to at least partially regulate the biological activity of said protein by inducing or restricting the conformation of the protein, said protein having first, second and third respective series of amino acid residues therein, said first series of amino acid residues forming a part of a switch control ligand and being individually modifiable in vivo between two respective states, said first series of amino acid residues binding with said second series of amino acid residues when the first series is in one of said states thereof, said first series of amino acid residues binding with said third series of amino acid residues when the first series is in the other of said states thereof, said molecule being bound to at least one of the amino acid residues of said first, second or third series.

15. The adduct of claim 14, all of the residues making up said first series being substantially simultaneously modifiable between said two respective states.

16. The adduct of claim 14, certain of said amino acid residues of said first series being modifiable separately from other amino acids of the first series.

17. The adduct of claim 14, the binding of said first series of amino acid residues with said second series of amino acid residues corresponding to a first conformation of said protein, and the binding of said first series of amino acid residues with said third series of amino acid residues corresponding to a second, different conformation of said protein.

18. The adduct of claim 14, said first series of amino acid residues being modifiable by phosphorylation, sulfation, fatty acid acylation, prenylation, glycosylation, carboxylation, nitrosylation, cystinylation, oxidation, or mutation.

19. The adduct of claim 18, each of the first series of amino acid residues being modifiable by phosphorylation, sulfation, fatty acid acylation, prenylation, glycosylation, carboxylation, nitrosylation, cystinylation, oxidation, or mutation wherein each of the first series of residues are modifiable by the same modality.

20. The adduct of claim 18, at least one of said first series of amino acid residues being modifiable by phosphorylation, sulfation, fatty acid acylation, prenylation, glycosylation, carboxylation, nitrosylation, cystinylation, oxidation, or mutation, and another of said first series of amino acid residues being modifiable by a process different than said at least one amino acid residue.

21. The adduct of claim 18, said molecule binding with at least one amino acid residue of said second or third series.

22. The adduct of claim 14, at least one of the amino acid residues of said first series being transiently modifiable.

23. The adduct of claim 14, at least one of the amino acid residues of said first series being substantially permanently modifiable.

24. The adduct of claim 14, said molecule binding with an amino acid residue of said first series thereof, and, as a part of said binding, chemically modifying the residue of the first series causing said residue to change its switch state.

25. The adduct of claim 24, said amino acid residue of said first series being modifiable by nitrosylation thereof, said molecule removing a nitrosyl group therefrom.

26. The adduct of claim 24, said amino acid residue of said first series being modifiable by oxidation thereof, said molecule removing an oxygen radical therefrom.

27. The adduct of claim 14, said molecule binding with an amino acid residue of said first series thereof, and, as a part of said binding, chemically modifying the residue by addition of a modifying moiety, in order to cause the residue to change its state.

28. The adduct of claim 27, said molecule oxidizing said amino acid residue of said first series thereof.

29. The adduct of claim 14, said first series of amino acid residues including a mutated, irreversibly modified amino acid residue.

30. The adduct of claim 14, said molecule binding with at least one amino acid residue of said second or third series.

31. The adduct of claim 14, said molecule binding with a plurality of residues of said first, second or third series.

32. The adduct of claim 31, said molecule binding with a plurality of residues of said second or third series.

33. The adduct of claim 14, said molecule binding with one or more residues of said second series.

34. The adduct of claim 14, said molecule binding with one or more residues of said third series.

35. The adduct of claim 14, said protein being a kinase protein.

36. The adduct of claim 35, said first series of amino acid residues being modifiable by phosphorylation, fatty acid acylation, nitrosylation, cystinylation, oxidation, or mutation.

37. The adduct of claim 36, each of the first series of amino acid residues being modifiable by phosphorylation, fatty acid acylation, nitrosylation, cystinylation, oxidation, or mutation wherein each of the first series of residues are modifiable by the same modality.

38. The adduct of claim 36, at least one of said first series of amino acid residues being modifiable by phosphorylation, fatty acid acylation, nitrosylation, cystinylation, oxidation, or mutation, and another of said first series of amino acid residues being modifiable by a process different than said at least one amino acid residue.

39. The adduct of claim 36, said molecule binding with at least one amino acid residue of said first series.

40. The adduct of claim 36, at least one of the amino acid residues of said first series being transiently modifiable.

41. The adduct of claim 36, at least one of the amino acid residues of said first series being substantially permanently modifiable.

42. The adduct of claim 36, said molecule binding with an amino acid residue of said first series thereof, and, as a part of said binding, chemically modifying the residue of the first series causing said residue to change its switch state.

43. The adduct of claim 42, said amino acid residue of said first series being modifiable by nitrosylation thereof, said molecule removing a nitrosyl group therefrom.

44. The adduct of claim 42, said amino acid residue of said first series being modifiable by oxidation thereof, said molecule removing an oxygen radical therefrom.

45. The adduct of claim 36, said molecule binding with an amino acid residue of said first series thereof, and, as a part of said binding, chemically modifying the residue by addition of a modifying moiety, in order to cause the residue to change its state.

46. The adduct of claim 45, said molecule oxidizing said amino acid residue of said first series thereof.

47. The adduct of claim 36, said first series of amino acid residues including a mutated, irreversibly modified amino acid residue.

48. The adduct of claim 36, said molecule binding with at least one amino acid residue of said second or third series.

49. The adduct of claim 36, said molecule binding with a plurality of residues of said first, second or third series.

50. The adduct of claim 49, said molecule binding with a plurality of residues of said second or third series.

51. The adduct of claim 36, said molecule binding with one or more residues of said second series.

52. The adduct of claim 36, said molecule binding with one or more residues of said third series.

53. The adduct of claim 35, said kinase protein being p38-alpha kinase.

54. The adduct of claim 53, said molecule binding to either arginine 70 and/or arginine 67 of said protein.

55. The adduct of claim 54, said molecule binding with the guanidyl moiety of either arginine 70 or arginine 67.

56. The adduct of claim 53, said molecule further binding with at least certain residues selected from the group consisting of glutamic acid 71, leucine 74, methionine 78, valine 83, isoleucine 141, histidine 148, phenylalanine 169, lysine 53, isoleucine 84, leucine 104, and threonine 106.

57. The adduct of claim 55, said molecule further binding with at least certain residues selected from the group consisting of glutamic acid 71, leucine 74, methionine 78, valine 83, isoleucine 141, histidine 148, phenylalanine 169, lysine 53, isoleucine 84, leucine 104, and threonine 106.

58. The adduct of claim 53, said molecule binding with tyrosine 35.

59. The adduct of claim 58, said molecule further binding with either arginine 70 and/or arginine 67, and at least certain residues selected from the group consisting of glutamic acid 71, leucine 74, methionine 78, valine 83, isoleucine 141, histidine 148, phenylalanine 169, lysine 53, isoleucine 84, leucine 104, and threonine 106.

60. The adduct of claim 35, said kinase being wild-type Braf kinase.

61. The adduct of claim 60, said molecule binding with one or more of asparagine 499, lysine 600, and arginine 602.

62. The adduct of claim 61, said molecule further binding with at least certain residues selected from the group consisting of alanine 496, valine 599, glutamic acid 500, phenylalanine 594, lysine 482, leucine 513, isoleucine 526, threonine 528, valine 503, leucine 504, threonine 507, isoleucine 512, leucine 566, and histidine 573.

63. The adduct of claim 35, said kinase being oncogenic V599E Braf kinase.

64. The adduct of claim 63, said molecule binding with one or more of asparagine 499, lysine 600, and arginine 602.

65. The adduct of claim 64, said molecule further binding with at least certain residues selected from the group consisting of alanine 496, glutamic acid 599, glutamic acid 500, phenylalanine 594, lysine 482, leucine 513, isoleucine 526, threonine 528, valine 503, leucine 504, threonine 507, isoleucine 512, leucine 566, and histidine 573.

66. The adduct of claim 63, said molecule binding with glutamic acid 599.

67. The adduct of claim 66, said molecule further binding with one or more of asparagine 499, lysine 600, and arginine 602, and at least certain residues selected from the group consisting of alanine 496, glutamic acid 599, glutamic acid 500, phenylalanine 594, lysine 482, leucine 513, isoleucine 526, threonine 528, valine 503, leucine 504, threonine 507, isoleucine 512, leucine 566, and histidine 573.

68. The adduct of claim 35, said protein being wild-type c-Abl kinase.

69. The adduct of claim 68, said molecule binding with one or more of arginine 405 or glutamic acid 301.

70. The adduct of claim 69, said molecule further binding with one or more of glutamic acid 305, phenylalanine 401, lysine 290, valine 318, isoleucine 332, threonine 334, valine 308, isoleucine 312, leucine 317, leucine 373, and histidine 380.

71. The adduct of claim 35, said protein being oncogenic Bcr-Abl kinase.

72. The adduct of claim 71, said molecule binding with one or more of arginine 405 or glutamic acid 301.

73. The adduct of claim 72, said molecule further binding with one or more of glutamic acid 305, phenylalanine 401, lysine 290, valine 318, isoleucine 332, threonine 334, valine 308, isoleucine 312, leucine 317, leucine 373, and histidine 380.

74. A protein-modulator adduct comprising p38-alpha kinase and a non-naturally occurring molecule bound to said kinase, said molecule binding to either arginine 70 and/or arginine 67 of said protein.

75. The adduct of claim 74, said molecule binding with the guanidyl moiety of either arginine 70 or arginine 67.

76. The adduct of claim 74, said molecule further binding with at least certain residues selected from the group consisting of glutamic acid 71, leucine 74, methionine 78, valine 83, isoleucine 141, histidine 148, phenylalanine 169, lysine 53, isoleucine 84, leucine 104, and threonine 106.

77. The adduct of claim 75, said molecule further binding with at least certain residues selected from the group consisting of glutamic acid 71, leucine 74, methionine 78, valine 83, isoleucine 141, histidine 148, phenylalanine 169, lysine 53, isoleucine 84, leucine 104, and threonine 106.

78. A protein-modulator adduct comprising p38-alpha kinase and a non-naturally occurring molecule bound to said kinase, said molecule binding with tyrosine 35.

79. The adduct of claim 78, said molecule further binding with either arginine 70 and/or arginine 67, and at least certain residues selected from the group consisting of glutamic acid 71, leucine 74, methionine 78, valine 83, isoleucine 141, histidine 148, phenylalanine 169, lysine 53, isoleucine 84, leucine 104, and threonine 106.

80. A protein-modulator adduct comprising wild-type Braf kinase and a non-naturally occurring molecule bound to said kinase, said molecule binding with one or more of asparagine 499, lysine 600, and arginine 602.

81. The adduct of claim 80, said molecule further binding with at least certain residues selected from the group consisting of alanine 496, valine 599, glutamic acid 500, phenylalanine 594, lysine 482, leucine 513, isoleucine 526, threonine 528, valine 503, leucine 504, threonine 507, isoleucine 512, leucine 566, and histidine 573.

82. A protein-modulator adduct comprising oncogenic V599E Braf kinase and a non-naturally occurring molecule bound to said kinase, said molecule binding with one or more of asparagine 499, lysine 600, and arginine 602.

83. The adduct of claim 82, said molecule further binding with at least certain residues selected from the group consisting of alanine 496, glutamic acid 599, glutamic acid 500, phenylalanine 594, lysine 482, leucine 513, isoleucine 526, threonine 528, valine 503, leucine 504, threonine 507, isoleucine 512, leucine 566, and histidine 573.

84. A protein-modulator adduct comprising oncogenic V599E Braf kinase and a non-naturally occurring molecule bound to said kinase, said molecule binding with glutamic acid 599.

85. The adduct of claim 84, said molecule further binding with one or more of asparagine 499, lysine 600, and arginine 602, and at least certain residues selected from the group consisting of alanine 496, glutamic acid 500, phenylalanine 594, lysine 482, leucine 513, isoleucine 526, threonine 528, valine 503, leucine 504, threonine 507, isoleucine 512, leucine 566, and histidine 573.

86. A protein-modulator adduct comprising wild-type Abl kinase and a non-naturally occurring molecule bound to said kinase, said molecule binding with arginine 405 or glutamic acid 301.

87. The adduct of claim 86, said molecule further binding with one or more of glutamic acid 305, phenylalanine 401, lysine 290, valine 318, isoleucine 332, threonine 334, valine 308, isoleucine 312, leucine 317, leucine 373, and histidine 380.

88. A protein-modulator adduct comprising oncogenic Bcr-Abl kinase and a non-naturally occurring molecule bound to said kinase, said molecule binding with arginine 405 or glutamic acid 301.

89. The adduct of claim 88, said molecule further binding with one or more of glutamic acid 305, phenylalanine 401, lysine 290, valine 318, isoleucine 332, threonine 334, valine 308, isoleucine 312, leucine 317, leucine 373, and histidine 380.

90. A protein-modulator adduct comprising caspase-7 dimer protease having chains A and B, and a non-naturally occurring molecule bound to said protease, said molecule binding with cysteine 290 from chain A of the dimer protease, and/or cysteine 290-prime from chain B of the dimer protease.

91. A protein-modulator adduct comprising caspase-7 dimer protease having chains A and B, and a non-naturally occurring molecule bound to said protease, said molecule binding with S-nitrosyl cysteine 290 from chain A of the dimer protease, and/or S-nitrosyl cysteine 290-prime from chain B of the dimer protease.

92. A method of altering the biological activity of a protein comprising the steps of providing a naturally occurring protein having a switch control pocket and a switch control ligand, said switch control pocket having a plurality of conformational control amino acid residues which are capable of binding with corresponding residues forming a part of said switch control ligand;
contacting said protein with a non-naturally occurring molecule modulator, and
causing said modulator to bind with said protein at the region of said pocket in order to at least partially regulate the biological activity of the protein by inducing or restricting the conformation of the protein, said molecule being bound to at least one amino acid residue taken from the switch pocket conformational control amino acid residues or the switch control ligand modifiable amino acid residues.

93. A method of altering the biological activity of a protein comprising the steps of
providing a naturally occurring protein having said protein having first, second and third respective series of amino acid residues therein, said first series of amino acid residues forming a part of a ligand and being individually modifiable in vivo between two respective states, said first series of amino acid residues binding with said second series of amino acid residues when the first series is in one of said states thereof, said first series of amino acid residues binding with said third series of amino acid residues when the first series is in the other of said states thereof;
contacting said protein with a non-naturally occurring modulator molecule; and
causing said modulator to bind with said protein in order to at least partially regulate the biological activity of the protein by inducing or restricting the conformation of the protein, said molecule being bound to at least one of the amino acid residues of said first, second or third series.

94. A method of claim 92, wherein contacting said protein with the non-naturally occurring molecule modulator induces, synergizes, or promotes the binding of a second small molecule modulator of said protein to form a ternary adduct, such co-incident binding resulting in enhanced biological modulation of the protein when compared to the biological modulation of the protein affected by either small molecule alone.

95. A method of claim 94, wherein the second small molecule interacts at a substrate, cofactor, or regulatory site on the protein, said second site being distinct from the switch control pocket that interacts with the first non-naturally occurring molecule modulator.

96. A method of claim 94, wherein the second small molecule interacts at an allosteric site on the protein, said second site being distinct from the switch control pocket that interacts with the first non-naturally occurring molecule modulator.

97. A method of claim 95, wherein the second small molecule interacts at an ATP cofactor site.

98. A method of claim 97, wherein the protein is a kinase.

99. A method of claim 98, wherein the kinase is c-Abl kinase or Bcr-Abl kinase.

100. A method of claim 99, wherein the second small molecule is taken from N-(4-methyl-3-(4-(pyridin-3-yl)pyrimidin-2-ylamino)phenyl)-4-((4-methylpiperazin-1-yl)methyl)benzamide(Gleevec); N-(2-chloro-6-methylphenyl)-2-(6-(4-(2-hydroxyethyl)piperazin-1-yl)-2-methylpyrimidin-4-ylamino)thiazole-5-carboxamide (BMS-354825); 6-(2,6-dichlorophenyl)-2-(3-(hydroxymethyl)phenylamino)-8-methylpyrido[2,3-d]pyrimidin-7(8H)-one (PD 166326); 6-(2,6-dichlorophenyl)-8-methyl-2-(3-(methylthio)phenylamino)pyrido[2,3-d]pyrimidin-7(8H)-one (PD 173955); 6-(2,6-dichlorophenyl)-2-(4-fluoro-3-methylphenylamino)-8-methylpyrido[2,3-d]pyrimidin-7(8H)-one (PD180970); 6-(2,6-dichlorophenyl)-2-(4-ethoxyphenylamino)-8-methylpyrido[2,3-d]pyrimidin-7(8H)-one (PD 173958); 6-(2,6-dichlorophenyl)-2-(4-fluorophenylamino)-8-methylpyrido[2,3-d]pyrimidin-7(8H)-one (PD 173956); 6-(2,6-dichlorophenyl)-2-(4-(2-(diethylamino)ethoxy)phenylamino)-8-methylpyrido[2,3-d]pyrimidin-7(8H)-one (PD 166285); 2-(4-(2-aminoethoxy)phenylamino)-6-(2,6-dichlorophenyl)-8-methylpyrido[2,3-d]pyrimidin-7(8H)-one; N-(3-(6-(2,6-dichlorophenyl)-8-methyl-7-oxo-7,8-dihydropyrido[2,3-d]pyrimidin-2-ylamino)phenyl)acetamide (SKI DV-MO 16); 2-(4-aminophenylamino)-6-(2,6-dichlorophenyl)-8-methylpyrido[2,3-d]pyrimidin-7(8H)-one (SKI DV 1-10); 6-(2,6-dichlorophenyl)-2-(3-hydroxyphenylamino)-8-methylpyrido[2,3-d]pyrimidin-7(8H)-one (SKI DV2-89); 2-(3-aminophenylamino)-6-(2,6-dichlorophenyl)-8-methylpyrido[2,3-d]pyrimidin-7(8H)-one (SKI DV 243); N-(4-(6-(2,6-dichlorophenyl)-8-methyl-7-oxo-7,8-dihydropyrido[2,3-d]pyrimidin-2-ylamino)phenyl)acetamide (SKI DV-M017); 6-(2,6-dichlorophenyl)-2-(4-hydroxyphenylamino)-8-methylpyrido[2,3-d]pyrimidin-7(8H)-one (SKI DV-M017); 6-(2,6-dichlorophenyl)-2-(3-ethylphenylamino)-8-methylpyrido[2,3-d]pyrimidin-7(8H)-one (SKI DV 2 87).

101. A method of claim 93, wherein contacting said protein with the non-naturally occurring molecule modulator induces, synergizes, or promotes the binding of a second small molecule modulator of said protein to form a ternary adduct, such co-incident binding resulting in enhanced biological modulation of the protein when compared to the biological modulation of the protein affected by either small molecule alone.

102. A method of claim 101, wherein the second small molecule interacts at a substrate, cofactor, or regulatory site on the protein, said second site being distinct from the switch control pocket that interacts with the first non-naturally occurring molecule modulator.

103. A method of claim 101, wherein the second small molecule interacts at an allosteric site on the protein, said second site being distinct from the switch control pocket that interacts with the first non-naturally occurring molecule modulator.

104. A method of claim 102, wherein the second small molecule interacts at an ATP cofactor site.

105. A method of claim 104, wherein the protein is a kinase.

106. A method of claim 105, wherein the kinase is c-Abl kinase or Bcr-Abl kinase.

107. A method of claim 106, wherein the second small molecule is taken from N-(4-methyl-3-(4-(pyridin-3-yl)pyrimidin-2-ylamino)phenyl)-4-((4-methylpiperazin-1-yl)methyl)benzamide(Gleevec); N-(2-chloro-6-methylphenyl)-2-(6-(4-(2-hydroxyethyl)piperazin-1-yl)-2-methylpyrimidin-4-ylamino)thiazole-5-carboxamide (BMS-354825); 6-(2,6-dichlorophenyl)-2-(3-(hydroxymethyl)phenylamino)-8-methylpyrido[2,3-d]pyrimidin-7(8H)-one (PD 166326); 6-(2,6-dichlorophenyl)-8-methyl-2-(3-(methylthio)phenylamino)pyrido[2,3-d]pyrimidin-7(8H)-one (PD 173955); 6-(2,6-dichlorophenyl)-2-(4-fluoro-3-methylphenylamino)-8-methylpyrido[2,3-d]pyrimidin-7(8H)-one (PD180970); 6-(2,6-dichlorophenyl)-2-(4-ethoxyphenylamino)-8-methylpyrido[2,3-d]pyrimidin-7(8H)-one (PD 173958); 6-(2,6-dichlorophenyl)-2-(4-fluorophenylamino)-8-methylpyrido[2,3-d]pyrimidin-7(8H)-one (PD 173956); 6-(2,6-dichlorophenyl)-2-(4-(2-(diethylamino)ethoxy)phenylamino)-8-methylpyrido[2,3-d]pyrimidin-7(8H)-one (PD 166285); 2-(4-(2-aminoethoxy)phenylamino)-6-(2,6-dichlorophenyl)-8-methylpyrido[2,3-d]pyrimidin-7(8H)-one; N-(3-(6-(2,6-dichlorophenyl)-8-methyl-7-oxo-7,8-dihydropyrido[2,3-d]pyrimidin-2-ylamino)phenyl)acetamide (SKI DV-MO 16); 2-(4-aminophenylamino)-6-(2,6-dichlorophenyl)-8-methylpyrido[2,3-d]pyrimidin-7(8H)-one (SKI DV 1-10); 6-(2,6-dichlorophenyl)-2-(3-hydroxyphenylamino)-8-methylpyrido[2,3-d]pyrimidin-7(8H)-one (SKI DV2-89); 2-(3-aminophenylamino)-6-(2,6-dichlorophenyl)-8-methylpyrido[2,3-d]pyrimidin-7(8H)-one (SKI DV 243); N-(4-(6-(2,6-dichlorophenyl)-8-methyl-7-oxo-7,8-dihydropyrido[2,3-d]pyrimidin-2-ylamino)phenyl)acetamide (SKI DV-MO17); 6-(2,6-dichlorophenyl)-2-(4-hydroxyphenylamino)-8-methylpyrido[2,3-d]pyrimidin-7(8H)-one (SKI DV-MO17); 6-(2,6-dichlorophenyl)-2-(3-ethylphenylamino)-8-methylpyrido[2,3-d]pyrimidin-7(8H)-one (SKI DV 2 87).
